(19) United States
(12) Patent Application Publication
(10) Pub. No.: US 2010/0099196 A1
(43) Pub. Date: Apr. 22, 2010

(54) PROCESS FOR NORMALIZING THE CONCENTRATION OF ANALYTES IN A URINE SAMPLE

(76) Inventor: Harald Mischak, Sehnde (DE)

Correspondence Address: DARDI & HERBERT, PLLC, 220 S. 6TH ST., SUITE 2000, U.S. BANK PLAZA, MINNEAPOLIS, MN, 55402, US

(21) Appl. No.: 12/530,029

(22) PCT Filed: Mar. 6, 2008
(86) PCT No.: PCT/EP2008/052730

§ 371 Date: Jan. 4, 2010

(30) Foreign Application Priority Data
Mar. 7, 2007 (EP) 07103684.2

(51) Int. Cl. G01N 33/53

(52) U.S. Cl. 436/86

(57) ABSTRACT

Process for normalizing the concentration of at least one analyte in a urine sample, comprising the following steps:
a) determining the concentration of at least one analyte;
b) determining the concentration of at least one collagen or collagen fragment;
c) normalizing the concentration of said at least one analyte relative to the concentration of said at least one collagen or collagen fragment.

The present invention relates to a process for normalizing the concentration of analytes in a urine sample.

Urine samples have been used for medical laboratory examinations for a long time. Urine samples contain a variety of substances that are interesting under medical aspects, for example, degradation products of medicaments or illegal drugs, hormones, pathogens, proteins etc.

While often (only) the total protein content was recurred to for evaluating a urine sample in the past, it is recognized more and more that the composition of the proteins in urine, in particular, also contains relevant medical information.

WO 01/84140 and WO 2004/068130 describe Processes and devices for determining protein and peptide patterns in a liquid sample, for example, a urine sample.

WO 2005/024409 describes devices and processes for the quantitative evaluation of the polypeptides contained in a sample of body fluids and markers for recognizing pathological conditions. For example, it is shown how different diseases are associated with the occurrence of different polypeptide markers. It is observed that different polypeptides occur more or less frequently in certain diseases as compared to healthy patients.

WO 2006/106129 among others describes so-called amplitude markers in which two states, for example states of health and disease, are distinguished from one another, not by the occurrence, but by the amplitude, i.e., the concentration, of the markers in the sample. In order to compensate for different protein concentrations of different samples, the overall amplitude is normalized to 1 million counts after an examination of the sample by mass spectrometry in WO 2006/106129.

However, there is still a need for more specific normalization procedures for comparing different concentrations of analytes in the sample and therefore achieve a better comparability between the reference samples and measured samples.

Surprisingly, this object can be achieved by a process for normalizing the concentration of at least one analyte in a urine sample, comprising the following steps:
a) determining the concentration of at least one analyte;
b) determining the concentration of at least one collagen or collagen fragment;
c) normalizing the concentration of said at least one analyte relative to the concentration of said at least one collagen or collagen fragment.

The process according to the invention has a wide variety of applications.

First, the content of analytes, collagen or collagen fragments is determined. For the determination, for example, UV/Vis detectors, fluorescence detectors, refractive index detectors, diode array detectors, mass spectrometers, gel scanners, the staining of the proteins, for example, with Coomassie brilliant blue, silver staining, Cy2, Cy3, Cy5 or the like, or the derivatization, for example, with ITRAC (isotope tags for relative and absolute qualification) and ICAT (isotope coded affinity tagging) followed by detection of the derivatized peptides may be used.

According to the invention, collagen or collagen fragments are employed for normalizing the samples. At least one collagen or collagen fragment is identified in the sample. For this purpose, identification by migration and retention times, in particular, optionally completed by mass spectra, UV/Vis spectra, fluorescence spectra or the affinity for particular ligands, for example, specific antibodies, is suitable.

The concentration of the analyte can then be normalized to the concentration of said at least one collagen or collagen fragment, i.e., the concentrations of analytes are measured relative to the concentration of the reference collagen or collagen fragments.

Preferably, however, not one collagen or collagen fragment, but several, for example, two or three or four or five or 10, 20, 30, 40 or 50 collagens or collagen fragments are employed for normalization.

In a preferred embodiment, the analyte does not comprise any hydroxypyridinium collagen cross-links, especially no hydroxylysylpyridinoline (Pyd) and deoxypyridinoline (Dpd). Preferably, none of these compounds is employed for normalizing the concentration of another analyte either.

Preferably used collagen fragments are stated in the following Table:

| Seq. ID No. | Protein name | Swissprot name | Accession number | Calc. mass [M] |
|---|---|---|---|---|
| 1 | Collagen alpha-1 (I) chain [777-787] [Homo sapiens] | CO1A1 Human | gi115269 | 1016.441 |
| 2 | Collagen alpha-1 (I) chain [546-558] [Homo sapiens] | CO1A1 Human | gi115269 | 1194.552 |
| 3 | Collagen alpha-1 (I) chain [798-810] [Homo sapiens] | CO1A1 Human | gi115269 | 1250.553 |
| 4 | Collagen alpha-1 (I) chain [546-559] [Homo sapiens] | CO1A1 Human | gi115269 | 1265.589 |
| 5 | Collagen alpha-1 (III) chain [796-808] [Homo sapiens] | CO3A1 Human | gi115306 | 1268.563 |
| 6 | Collagen alpha-1 (II) chain [511-524] [Homo sapiens] | CO2A1 Human | gi115287 | 1378.611 |
| 7 | Collagen alpha-1 (I) chain [543-558] [Homo sapiens] | CO1A1 Human | gi115269 | 1435.658 |
| 8 | Collagen alpha-1 (III) chain [642-657] [Homo sapiens] | CO3A1 Human | gi115306 | 1438.669 |
| 9 | Collagen alpha-1 (I) chain [543-558] [Homo sapiens] | CO1A1 Human | gi115269 | 1451.653 |
| 10 | Collagen alpha-1 (I) chain [542-558] [Homo sapiens] | CO1A1 Human | gi115269 | 1508.674 |
| 11 | Collagen alpha-1 (XV) chain [1093-1109] [Homo sapiens] | COFA1 Human | gi68839886 | 1523.726 |
| 12 | Collagen alpha-1 (I) chain [543-560] [Homo sapiens] | CO1A1 Human | gi115269 | 1579.711 |
| 13 | Collagen alpha-1 (III) chain [587-604] [Homo sapiens] | CO3A1 Human | gi115306 | 1623.724 |
| 14 | Collagen alpha-1 (I) chain [541-560] [Homo sapiens] | CO1A1 Human | gi115269 | 1737.781 |
| 15 | Collagen alpha-1 (III) chain [642-661] [Homo sapiens] | CO3A1 Human | gi115306 | 1834.833 |
| 16 | Collagen alpha-1 (III) chain [1174-1195] [Homo sapiens] | CO3A1 Human | gi115306 | 2025.866 |
| 17 | Collagen alpha-1 (I) chain [648-669] [Homo sapiens] | CO1A1 Human | gi115269 | 2046.949 |
| 18 | Collagen alpha-1 (I) chain [229-249] [Homo sapiens] | CO1A1 Human | gi115269 | 2063.926 |
| 19 | Collagen alpha-1 (III) chain [586-604] [Homo sapiens] | CO3A1 Human | gi115306 | 2078.925 |
| 20 | Collagen alpha-1 (I) chain [647-669] [Homo sapiens] | CO1A1 Human | gi115269 | 2087.976 |
| 21 | Collagen alpha-1 (I) chain [647-669] [Homo sapiens] | CO1A1 Human | gi115269 | 2103.971 |
| 22 | Collagen alpha-1 (I) chain [646-669] [Homo sapiens] | CO1A1 Human | gi115269 | 2159.013 |
| 23 | Collagen alpha-1 (I) chain [432-455] [Homo sapiens] | CO1A1 Human | gi115269 | 2169.977 |
| 24 | Collagen alpha-1 (I) chain [646-669] [Homo sapiens] | CO1A1 Human | gi115269 | 2175.008 |
| 25 | Collagen alpha-1 (I) chain [819-843] [Homo sapiens] | CO1A1 Human | gi115269 | 2220.988 |
| 26 | Collagen alpha-1 (I) chain [431-455] [Homo sapiens] | CO1A1 Human | gi115269 | 2226.999 |
| 27 | Collagen alpha-1 (I) chain [699-725] [Homo sapiens] | CO1A1 Human | gi115269 | 2281.979 |
| 28 | Collagen alpha-1 (I) chain [819-844] [Homo sapiens] | CO1A1 Human | gi115269 | 2292.025 |
| 29 | Collagen alpha-1 (III) chain [813-840] [Homo sapiens] | CO3A1 Human | gi115306 | 2564.149 |
| 30 | Collagen alpha-1 (III) chain [1168-1195] [Homo sapiens] | CO3A1 Human | gi115306 | 2679.202 |
| 31 | Collagen alpha-1 (III) chain [1168-1195] [Homo sapiens] | CO3A1 Human | gi115306 | 2695.197 |
| 32 | Collagen alpha-1 (III) chain [610-639] [Homo sapiens] | CO3A1 Human | gi115306 | 2742.248 |
| 33 | Collagen alpha-1 (III) chain [448-477] [Homo sapiens] | CO3A1 Human | gi115306 | 2825.270 |
| 34 | Collagen alpha-1 (I) chain [1011-1041] [Homo sapiens] | CO1A1 Human | gi115269 | 2942.299 |
| 35 | Collagen alpha-1 (I) chain [537-569] [Homo sapiens] | CO1A1 Human | gi115269 | 3011.386 |
| 36 | Collagen alpha-1 (I) chain [1011-1040] [Homo sapiens] | CO1A1 Human | gi115269 | 3013.336 |
| 37 | Collagen alpha-2 (I) chain [830-865] [Homo sapiens] | CO1A2 Human | gi82654930 | 3248.533 |
| 38 | Collagen alpha-1 (I) chain [1007-1041] [Homo sapiens] | CO1A1 Human | gi115269 | 3266.442 |
| 39 | Collagen alpha-1 (III) chain [910-948] [Homo sapiens] | CO3A1 Human | gi115306 | 3441.603 |
| 40 | Collagen alpha-1 (I) chain [630-674] [Homo sapiens] | CO1A1 Human | gi115269 | 4169.916 |
| 41 | Collagen alpha-1 (III) chain [319-366] [Homo sapiens] | CO3A1 Human | gi115306 | 4289.919 |
| 42 | Collagen alpha-1 (III) chain [319-366] [Homo sapiens] | CO3A1 Human | gi115306 | 4305.914 |

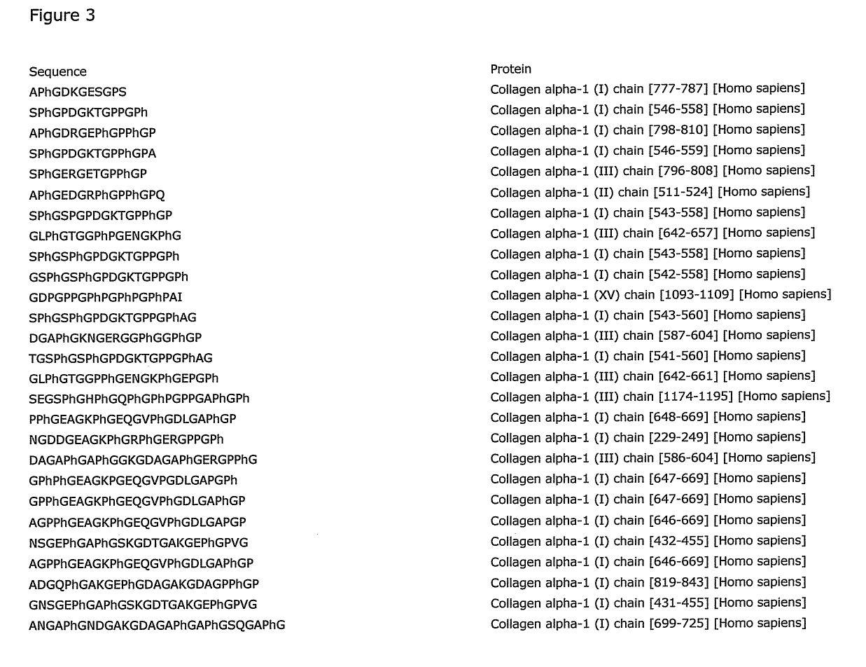

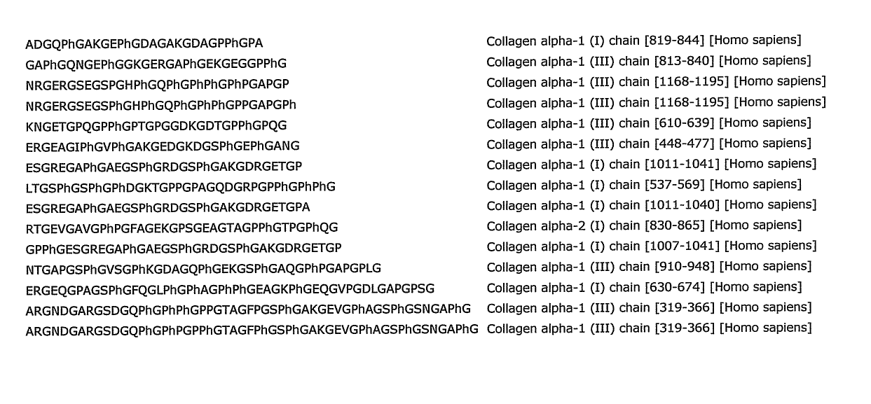

The sequences are given in FIG. 3.

The invention also relates to the use of collagen fragments for normalizing the analyte concentration of urine samples, especially with the collagen fragments as described above.

Preferred analytes whose contents are to be normalized include, in particular, proteins, peptides, lipids, metabolites, salts and other ionic compounds, carbohydrates.

The use of the process for normalizing peptide concentrations is particularly preferred.

In one embodiment, the sample is first subdivided into subsamples.

Different methods may be employed to achieve this, for example:
chromatographic methods, such as affinity chromatography, ion-exchange chromatography, reversed-phase chromatography, hydrophobic interaction chromatography, gel filtration, optionally using FPLC or HPLC;
adsorption to specific materials, such as SELDI process, ClinProt process;
electrophoretic methods, such as gel electrophoresis or capillary electrophoresis.

The invention is further illustrated by the following Examples:

### EXAMPLE 1

A urine sample was examined by means of HPLC with a downstream diode array detector (DAD). The raw date are stated in the Table. Peaks were identified by means of the retention time and spectra in the DAD. The measurement was compared with previously determined standards for these four collagen fragments, and a conversion factor was calculated for each. The average of these factors (0.80761277 in this case) was employed as a normalizing factor for all the other signals. The normalized signals are stated in the last column.

| Peak | | | Normalizing | Normalized |
|---|---|---|---|---|
| No. | Signal | Standard | factor | signal intensity |
| 1 | 126.8033157 | | | 157.0100429 |
| 2 | 146.5503894 | | | 181.4612086 |
| 3 | 3115.519875 | 4073.54509 | 0.76481782 | 3857.690207 |
| 4 | 590.1256269 | | | 730.7036846 |
| 5 | 5461.634925 | | | 6762.690148 |
| 6 | 150.3823428 | | | 186.2059991 |
| 7 | 39.1780827 | | | 48.51097474 |
| 8 | 8.5271778 | | | 10.55849798 |
| 9 | 394.6322538 | | | 488.6404331 |
| 10 | 50.0342319 | | | 61.9532451 |
| 11 | 1577.919488 | | | 1953.807005 |
| 12 | 539.3141163 | | | 667.7879996 |
| 13 | 400.4296182 | | | 495.8188292 |
| 14 | 8.8306578 | | | 10.93427212 |
| 15 | 148.7699163 | | | 184.209465 |
| 16 | 538.5410307 | | | 666.8307518 |
| 17 | 1389.860945 | | | 1720.949688 |
| 18 | 93.4206021 | | | 115.6749937 |
| 19 | 1024.846846 | 1276.56782 | 0.80281426 | 1268.982963 |
| 20 | 2344.443075 | | | 2902.929673 |
| 21 | 435.1965894 | | | 538.86789 |
| 22 | 114.2890587 | | | 141.514675 |
| 23 | 15.9397308 | | | 19.73684839 |
| 24 | 8.9629632 | | | 11.09809494 |
| 25 | 398.4975981 | | | 493.4265688 |
| 26 | 6940.8252 | 8643.25876 | 0.80303337 | 8594.248947 |
| 27 | 76.7856591 | | | 95.07732162 |
| 28 | 61.2618498 | | | 75.85547438 |
| 29 | 64.5422931 | | | 79.9173756 |
| 30 | 559.1427462 | | | 692.3401497 |
| 31 | 323.2918818 | | | 400.3055594 |
| 32 | 14.1058476 | | | 17.46610272 |
| 33 | 399.1654701 | | | 494.2535393 |
| 34 | 4612.278375 | | | 5711.002284 |
| 35 | 174.3985782 | | | 215.9433142 |
| 36 | 136.4525784 | | | 168.9579257 |
| 37 | 909.9711288 | | | 1126.741877 |
| 38 | 62.1722196 | | | 76.9827099 |
| 39 | 119.8605303 | | | 148.4133668 |
| 40 | 2922.828975 | 3399.48571 | 0.85978563 | 3619.097026 |
| 41 | 2.625588 | | | 3.251048147 |
| 42 | 52.8236532 | | | 65.40715445 |
| 43 | 178.5294621 | | | 221.0582456 |
| | | | 0.80761277 | |

### EXAMPLE 2

A urine sample was separated by means of 2D electrophoresis as described in Thongboonkerd et al. in Kidney Int 62: 1461-1469 (2002), over a pH range of from 3 to 10 and using a 10% polyacrylamide gel having a length of 22 cm. The gel was stained with Coomassie stain, and the collagens were detected in a gel prepared in parallel by means of immunological methods (Western blot). Predetermined standards for these five proteins were employed, and a linear regression performed. With this calibration formula, the further values were converted. The formula was Y=103.82x−127449. The R² value was 0.957.

| Peak | | | |
|---|---|---|---|
| No. | Signal | Standard | Normalized signal intensity |
| 1 | 4508518.988 | | 467946992.3 |
| 2 | 153935103 | | 15981414944 |
| 3 | 23582568 | | 2448214761 |
| 4 | 44251661.9 | | 4594080090 |
| 5 | 586248.746 | | 60736895.81 |
| 6 | 948640.866 | | 98360445.71 |
| 7 | 1383514.774 | | 143509054.8 |
| 8 | 122812941 | | 12750312086 |
| 9 | 2944789.572 | | 305600604.4 |
| 10 | 30550952.65 | | 3171672456 |
| 11 | 1089289.56 | 117284244.8 | 112962593.1 |
| 12 | 577112.586 | | 59788379.68 |
| 13 | 1130932.024 | | 117285913.7 |
| 14 | 913664.024 | | 94729149.97 |
| 15 | 397773.048 | | 41169348.84 |
| 16 | 6931703.714 | | 719522030.6 |
| 17 | 38342270.25 | | 3980567048 |
| 18 | 2391089.672 | | 248115480.7 |
| 19 | 2250316.422 | | 233500401.9 |
| 20 | 932726.014 | | 96708165.77 |
| 21 | 10695075.82 | | 1110235323 |
| 22 | 1438795.038 | | 149248251.8 |
| 23 | 8548926.298 | | 887422079.3 |
| 24 | 7982762.024 | | 828642904.3 |
| 25 | 519540.4375 | 58011688.21 | 53811239.22 |
| 26 | 4877082.25 | | 506211230.2 |
| 27 | 27564764.65 | | 2861646417 |
| 28 | 20622151.95 | | 2140864367 |
| 29 | 3862566.202 | | 400884174.1 |
| 30 | 219714.4865 | 23759647.58 | 22683308.99 |
| 31 | 13240495.55 | | 1374500799 |
| 32 | 6102716.5 | | 633456578 |
| 33 | 3523566.41 | | 365689215.7 |
| 34 | 1011312.708 | | 104867036.3 |
| 35 | 10681983.25 | | 1108876052 |
| 36 | 390792.574 | | 40444636.03 |
| 37 | 3552944.744 | | 368739274.3 |
| 38 | 8037397.154 | | 834315123.5 |
| 39 | 1966378.988 | | 204022017.5 |
| 40 | 89404658.25 | | 9281864171 |
| 41 | 2051887.75 | | 212899537.2 |
| 42 | 7424138.214 | | 770646580.4 |
| 43 | 32377626.4 | | 3361317724 |
| 44 | 34631829 | | 3595349038 |
| 45 | 1948609.006 | | 202177138 |
| 46 | 107055624.3 | | 11114387461 |
| 47 | 10008414.75 | | 1038946170 |
| 48 | 3575878.756 | | 371120283.4 |
| 49 | 1359764.006 | | 141043250.1 |
| 50 | 631532.685 | 52349499.17 | 65438274.36 |
| 51 | 1981878.154 | | 205631140.9 |
| 52 | 6658241.834 | | 691131218.2 |
| 53 | 8132593.25 | | 844198382.2 |
| 54 | 5853332.798 | | 607565562.1 |
| 55 | 10995087.2 | | 1141382504 |
| 56 | 3309612.762 | | 343476548 |
| 57 | 5692416.786 | | 590859261.7 |
| 58 | 3166113.976 | | 328578504 |
| 59 | 20333775.95 | | 2110925170 |
| 60 | 32900793.65 | | 3415632948 |
| 61 | 5041336.916 | | 523264149.6 |
| 62 | 2926167.976 | | 303667310.3 |
| 63 | 10445490.05 | | 1084323328 |
| 64 | 775850.282 | | 80421327.28 |
| 65 | 339214.044 | | 35089753.05 |
| 66 | 41363211.15 | | 4294201133 |
| 67 | 23456220.45 | | 2435097358 |
| 68 | 15788615 | | 1639046560 |
| 69 | 34321605.15 | | 3563141598 |
| 70 | 897196.084 | | 93019448.44 |
| 71 | 327241.543 | 37343483.31 | 33846767.99 |
| 72 | 9414104.178 | | 977244846.8 |
| 73 | 1965153.274 | | 203894763.9 |
| 74 | 45057999.65 | | 4677794075 |
| 75 | 877939.968 | | 91020278.48 |
| 76 | 3120269.268 | | 323818906.4 |
| 77 | 3584994.964 | | 372066728.2 |
| 78 | 10224568.98 | | 1061387302 |
| 79 | 735893.154 | | 76272978.25 |
| 80 | 5907069.334 | | 613144489.3 |
| 81 | 36156493.15 | | 3753639670 |
| 82 | 10877155.98 | | 1129138884 |
| 83 | 8374707.928 | | 869334728.1 |
| 84 | 16689498.2 | | 1732576254 |
| 85 | 5895062.464 | | 611897936 |
| 86 | 1706771.568 | | 177069575.2 |

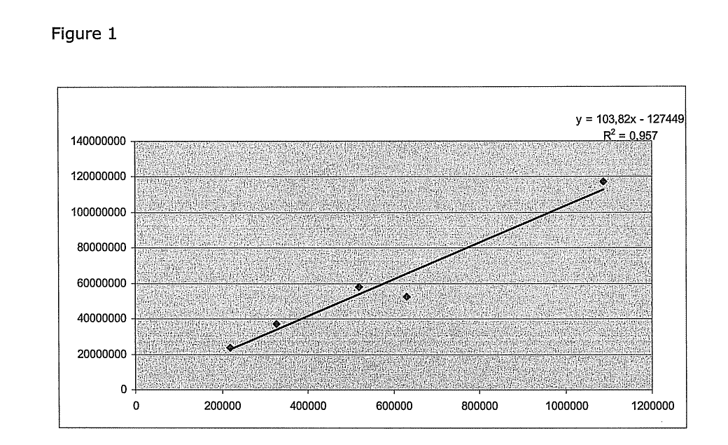

The quality of the regression is shown in FIG. 1.

### EXAMPLE 3

A urine sample was examined by capillary electrophoresis coupled with mass spectrometry. Twenty-seven collagen fragments could be identified thereby, which were then compared with previously determined standards. This was followed by normalization by means of linear regression with the proviso that the straight line must go through the value zero. Corresponding measuring data and the calibration are shown in the following Table.

| No. | CE time (min) | Mass | Signal | Standard | Normalized signal intensity |
|---|---|---|---|---|---|
| 1 | 19.948 | 17266.043 | 184882.922 | | 1368.13362 |
| 2 | 19.944 | 17119.789 | 587371.188 | | 4346.54679 |
| 3 | 19.931 | 17062.977 | 240560 | | 1780.144 |
| 4 | 19.946 | 16972.203 | 352392.875 | | 2607.70728 |
| 5 | 19.908 | 16952.465 | 197797.906 | | 1463.7045 |
| 6 | 19.931 | 16918.75 | 2107725.75 | | 15597.1706 |
| 7 | 19.873 | 16806.326 | 124057.313 | | 918.024116 |
| 8 | 19.917 | 16771.715 | 1365796.63 | | 10106.895 |
| 9 | 22.387 | 10342.543 | 119401.375 | | 883.570175 |
| 10 | 20.699 | 9905.148 | 89822.563 | | 664.686966 |
| 11 | 20.727 | 9867.076 | 526946.5 | | 3899.4041 |
| 12 | 20.599 | 9626.376 | 176806.484 | | 1308.36798 |
| 13 | 20.904 | 8875.947 | 124966.711 | | 924.753661 |
| 14 | 20.927 | 8837.85 | 796843.125 | | 5896.63913 |
| 15 | 19.926 | 8578.571 | 17265.779 | | 127.766765 |
| 16 | 20.156 | 8289.725 | 286028.781 | | 2116.61298 |
| 17 | 20.078 | 8176.791 | 674227.938 | | 4989.28674 |
| 18 | 57.332 | 6788.82 | 438.533 | | 3.2451442 |
| 19 | 19.251 | 6491.179 | 95395.688 | | 705.928091 |
| 20 | 23.865 | 6329.103 | 62460.586 | | 462.208336 |
| 21 | 19.542 | 6308.096 | 23674.088 | | 175.188251 |
| 22 | 20.842 | 6237.104 | 134147.797 | | 992.693698 |
| 23 | 23.72 | 6225.298 | 67887.742 | | 502.369291 |
| 24 | 23.66 | 6208.243 | 85896.305 | | 635.632657 |
| 25 | 23.741 | 6186.583 | 288461.313 | | 2134.61372 |
| 26 | 23.643 | 6170.012 | 399146.313 | | 2953.68272 |
| 27 | 19.662 | 5861.38 | 218662.969 | | 1618.10597 |
| 28 | 22.258 | 5635.462 | 14248.398 | | 105.438145 |
| 29 | 22.266 | 5612.497 | 112633.008 | | 833.484259 |
| 30 | 22.301 | 5574.535 | 988766.188 | | 7316.86979 |
| 31 | 20.377 | 5118.564 | 18909.668 | | 139.931543 |
| 32 | 20.441 | 5096.631 | 93874.516 | | 694.671418 |
| 33 | 20.448 | 5058.56 | 250174.453 | | 1851.29095 |
| 34 | 23.737 | 5039.495 | 283460.031 | | 2097.60423 |
| 35 | 22.982 | 5038.431 | 174797.016 | | 1293.49792 |
| 36 | 20.431 | 5036.265 | 628288.625 | | 4649.33583 |
| 37 | 23.001 | 5023.362 | 43507.188 | | 321.953191 |
| 38 | 20.438 | 5020.505 | 641471.938 | | 4746.89234 |
| 39 | 23.009 | 5000.172 | 753943.563 | | 5579.18237 |
| 40 | 20.45 | 4998.592 | 2524795 | | 18683.483 |
| 41 | 20.409 | 4982.02 | 313462.719 | | 2319.62412 |
| 42 | 20.549 | 4976.683 | 1167032.75 | | 8636.04235 |
| 43 | 52.301 | 4964.782 | 358.613 | | 2.6537362 |
| 44 | 20.466 | 4960.742 | 14400323 | | 106562.39 |
| 45 | 20.358 | 4933.646 | 23247.875 | | 1652.03428 |
| 46 | 22.651 | 4876.404 | 51850.109 | | 383.690807 |
| 47 | 38.197 | 4861.141 | 494.304 | | 3.6578496 |
| 48 | 22.682 | 4838.021 | 281694.156 | | 2084.53675 |
| 49 | 22.655 | 4822.251 | 52409.906 | | 387.833304 |
| 50 | 22.683 | 4800.063 | 1058541.25 | | 7833.20525 |
| 51 | 48.573 | 4793.838 | 282.485 | | 2.090389 |
| 52 | 55.222 | 4777.634 | 374.187 | | 2.7689838 |
| 53 | 39.763 | 4774.006 | 532.92 | | 3.943608 |
| 54 | 48.826 | 4772.17 | 272.009 | | 2.0128666 |
| 55 | 57.032 | 4766.609 | 339.383 | | 2.5114342 |
| 56 | 19.62 | 4730.656 | 24256.555 | | 179.498507 |
| 57 | 37.899 | 4719.636 | 2192.44 | | 16.224056 |
| 58 | 23.902 | 4692.252 | 68363.453 | | 505.889552 |
| 59 | 54.464 | 4690.256 | 358.075 | | 2.649755 |
| 60 | 22.332 | 4671.967 | 96398.766 | | 713.350868 |
| 61 | 25.889 | 4668.661 | 32946.504 | | 243.80413 |
| 62 | 21.078 | 4656.171 | 37012.164 | | 273.890014 |
| 63 | 23.915 | 4654.204 | 252635.781 | | 1869.50478 |
| 64 | 19.366 | 4651.778 | 422.893 | | 3.1294082 |
| 65 | 25.936 | 4630.038 | 76921.227 | | 569.21708 |
| 66 | 54.567 | 4620.673 | 895.427 | | 6.6261598 |
| 67 | 23.396 | 4619.578 | 16829.051 | | 124.534977 |
| 68 | 52.701 | 4602.985 | 298.8 | | 2.21112 |
| 69 | 58.14 | 4602.554 | 423.734 | | 3.1356316 |
| 70 | 57.637 | 4594.426 | 391.032 | | 2.8936368 |
| 71 | 52.603 | 4577.292 | 340.97 | | 2.523178 |
| 72 | 20.741 | 4566.496 | 232446.344 | | 1720.10295 |
| 73 | 49.024 | 4560.303 | 642.982 | | 4.7580668 |
| 74 | 54.011 | 4559.723 | 4928.111 | | 36.4680214 |
| 75 | 18.86 | 4552.372 | 345.112 | | 2.5538288 |
| 76 | 51.846 | 4548.482 | 373.052 | | 2.7605848 |
| 77 | 26.066 | 4547.384 | 12106.5 | | 89.5881 |
| 78 | 51.365 | 4544.345 | 1851.577 | | 13.7016698 |
| 79 | 53.986 | 4543.723 | 22582.416 | | 167.109878 |
| 80 | 54.024 | 4520.931 | 4122.547 | | 30.5068478 |
| 81 | 40.87 | 4520.884 | 552.198 | | 4.0862652 |
| 82 | 25.763 | 4468.206 | 33454.656 | | 247.564454 |
| 83 | 25.839 | 4452.583 | 21824.857 | | 161.503942 |
| 84 | 48.976 | 4445.646 | 571.726 | | 4.2307724 |
| 85 | 20.501 | 4443.026 | 18179.678 | | 134.529617 |
| 86 | 27.642 | 4439.813 | 97056.883 | | 718.220934 |
| 87 | 24.137 | 4436.181 | 404617.531 | | 2994.16973 |
| 88 | 58.694 | 4420.792 | 432.338 | | 3.1993012 |
| 89 | 25.813 | 4414.273 | 15183.697 | | 112.359358 |
| 90 | 19.965 | 4410.955 | 338675.063 | | 2506.19547 |
| 91 | 20.515 | 4405.046 | 291842.344 | | 2159.63335 |
| 92 | 25.558 | 4404.052 | 42032.641 | | 311.041543 |
| 93 | 20.055 | 4391.25 | 224259.906 | | 1659.5233 |
| 94 | 59.551 | 4390.04 | 363.251 | | 2.6880574 |
| 95 | 59.07 | 4383.307 | 677.349 | | 5.0123826 |
| 96 | 20.018 | 4375.046 | 105928.648 | | 783.871995 |
| 97 | 25.571 | 4366.142 | 216682.813 | | 1603.45282 |
| 98 | 20.066 | 4353.102 | 1819283.25 | | 13462.6961 |
| 99 | 22.383 | 4349.45 | 216404.641 | | 1601.39434 |
| 100 | 25.569 | 4344.106 | 288232.313 | | 2132.91912 |
| 101 | 20.337 | 4334.652 | 45377.477 | | 335.79333 |
| 102 | 25.603 | 4328.157 | 542624 | | 4015.4176 |
| 103 | 60.458 | 4319.592 | 330.163 | | 2.4432062 |
| 104 | 27.298 | 4307.342 | 30545.982 | | 226.040267 |
| 105 | 25.614 | 4306.212 | 529838.313 | 3100.93516 | 3920.80352 |
| 106 | 18.866 | 4302.308 | 277.252 | | 2.0516648 |
| 107 | 25.618 | 4290.197 | 708976.625 | 3872.09443 | 5246.42703 |
| 108 | 25.58 | 4274.179 | 57546.574 | | 425.844648 |
| 109 | 25.607 | 4252.228 | 98721.375 | | 730.538175 |
| 110 | 23.903 | 4239.119 | 31304.807 | | 231.655572 |
| 111 | 20.202 | 4233.57 | 31109.875 | | 230.213075 |
| 112 | 25.741 | 4229.222 | 27980.471 | | 207.055485 |
| 113 | 23.93 | 4217.721 | 698545.563 | | 5169.23717 |
| 114 | 28.392 | 4208.093 | 85557.367 | | 633.124516 |
| 115 | 20.629 | 4196.917 | 227998.453 | | 1687.18855 |
| 116 | 28.393 | 4192.087 | 152065.688 | | 1125.28609 |
| 117 | 27.019 | 4180.957 | 29489.236 | | 218.220346 |
| 118 | 28.396 | 4170.166 | 176045.016 | 1291.45231 | 1302.73312 |
| 119 | 23.8 | 4160.604 | 154588.094 | | 1143.9519 |
| 120 | 22.532 | 4160.021 | 318391.75 | | 2356.09895 |
| 121 | 27.033 | 4160.011 | 69551.938 | | 514.684341 |
| 122 | 28.349 | 4154.089 | 35176.504 | | 260.30613 |
| 123 | 27.009 | 4144.035 | 62706.73 | | 464.029802 |
| 124 | 22.513 | 4144.034 | 67626.914 | | 500.439164 |
| 125 | 23.294 | 4135.927 | 40501.199 | | 299.708873 |
| 126 | 22.526 | 4122.078 | 1111014.88 | | 8221.51008 |
| 127 | 27.052 | 4121.97 | 143200.75 | | 1059.68555 |
| 128 | 26.993 | 4106.017 | 56078.742 | | 414.982691 |
| 129 | 20.242 | 4105.763 | 27811.58 | | 205.805692 |
| 130 | 28.055 | 4102.563 | 18823.371 | | 139.292945 |
| 131 | 27.022 | 4084.008 | 103050.93 | | 762.576882 |
| 132 | 28.057 | 4082.969 | 35312.172 | | 261.310073 |
| 133 | 28.103 | 4063.103 | 70996.594 | | 525.374796 |
| 134 | 20.494 | 4062.357 | 192299.219 | | 1423.01422 |
| 135 | 20.46 | 4046.127 | 45463.988 | | 336.433511 |
| 136 | 20.849 | 4044.743 | 40488.367 | | 299.613916 |
| 137 | 28.08 | 4044.394 | 166345.453 | | 1230.95635 |
| 138 | 20.276 | 4044.247 | 575594.75 | | 4259.40115 |
| 139 | 20.865 | 4028.943 | 54097.566 | | 400.321988 |
| 140 | 20.601 | 4025.685 | 273715.438 | | 2025.49424 |
| 141 | 28.152 | 4025.156 | 75278.781 | | 557.062979 |
| 142 | 25.055 | 4015.3 | 13494.786 | | 99.8614164 |
| 143 | 20.423 | 4009.552 | 46446.355 | | 343.703027 |
| 144 | 20.88 | 4007.199 | 241439.844 | | 1786.65485 |
| 145 | 28.076 | 4006.013 | 98433.141 | | 728.405243 |
| 146 | 20.719 | 3996.902 | 309862.719 | | 2292.98412 |
| 147 | 20.438 | 3986.617 | 1697006.63 | | 12557.849 |
| 148 | 20.881 | 3968.808 | 427206.25 | | 3161.32625 |
| 149 | 19.8 | 3960.04 | 129275.078 | | 956.635577 |
| 150 | 19.704 | 3913.814 | 25934.791 | | 191.917453 |
| 151 | 19.645 | 3900.239 | 29670.904 | | 219.56469 |
| 152 | 19.631 | 3877.959 | 85786.75 | | 634.82195 |
| 153 | 19.619 | 3861.996 | 59334.113 | | 439.072436 |
| 154 | 23.401 | 3860.224 | 31707.752 | | 234.637365 |
| 155 | 19.105 | 3856.541 | 110644.117 | | 818.766466 |
| 156 | 28.332 | 3839.987 | 28982.951 | | 214.473837 |
| 157 | 19.659 | 3839.739 | 105607.102 | | 781.492555 |
| 158 | 25.36 | 3832.015 | 15375.597 | | 113.779418 |
| 159 | 23.419 | 3822.288 | 21333.066 | | 157.864688 |
| 160 | 20.048 | 3814.276 | 379764.75 | | 2810.25915 |
| 161 | 28.373 | 3801.977 | 68342.133 | | 505.731784 |
| 162 | 27.677 | 3801.896 | 13939.659 | | 103.153477 |
| 163 | 27.719 | 3794.861 | 51831.453 | | 383.552752 |
| 164 | 21.453 | 3782.819 | 17585.135 | | 130.129999 |
| 165 | 31.428 | 3775.772 | 53642.129 | | 396.951755 |
| 166 | 27.744 | 3772.916 | 129047.891 | | 954.954393 |
| 167 | 21.446 | 3760.889 | 121781.781 | | 901.185179 |
| 168 | 19.224 | 3760.068 | 42083.434 | | 311.417412 |
| 169 | 21.836 | 3757.834 | 12074.36 | | 89.350264 |
| 170 | 27.7 | 3756.982 | 327179.375 | | 2421.12738 |
| 171 | 21.886 | 3741.82 | 31431.242 | | 232.591191 |
| 172 | 27.715 | 3739.881 | 56333.398 | | 416.867145 |
| 173 | 27.678 | 3735.445 | 121638.703 | | 900.126402 |
| 174 | 19.018 | 3729.583 | 19175.627 | | 141.89964 |
| 175 | 21.454 | 3722.747 | 1143063.38 | | 8458.66898 |
| 176 | 21.905 | 3720.201 | 182391.875 | | 1349.69988 |
| 177 | 27.69 | 3718.93 | 211491.453 | | 1565.03675 |
| 178 | 19.466 | 3710.955 | 28338.004 | | 209.70123 |
| 179 | 21.427 | 3706.46 | 187850.703 | | 1390.0952 |
| 180 | 21.882 | 3703.562 | 156398.141 | | 1157.34624 |
| 181 | 27.706 | 3702.282 | 83424.43 | | 617.340782 |
| 182 | 27.527 | 3699.89 | 27447.908 | | 203.114519 |
| 183 | 33.259 | 3695.777 | 61599.129 | | 455.833555 |
| 184 | 21.69 | 3686.37 | 179506.266 | | 1328.34637 |
| 185 | 31.253 | 3674.799 | 30919.297 | | 228.802798 |
| 186 | 22.842 | 3669.826 | 17117.689 | | 126.670899 |
| 187 | 18.862 | 3662.674 | 745.133 | | 5.5139842 |
| 188 | 33.247 | 3657.754 | 144639.984 | | 1070.33588 |
| 189 | 21.345 | 3630.767 | 245992.188 | | 1820.34219 |
| 190 | 19.415 | 3620.771 | 139033.516 | | 1028.84802 |
| 191 | 28.076 | 3616.437 | 45247.488 | | 334.831411 |
| 192 | 19.346 | 3605.381 | 42433.102 | | 314.004955 |
| 193 | 26.967 | 3593.676 | 72137.609 | | 533.818307 |
| 194 | 19.373 | 3582.885 | 557225.625 | | 4123.46963 |
| 195 | 27.002 | 3571.638 | 158852.625 | | 1175.50943 |
| 196 | 27.423 | 3562.87 | 16845.193 | | 124.654428 |
| 197 | 23.372 | 3559.398 | 131749.125 | | 974.943525 |
| 198 | 22.789 | 3556.364 | 63784.465 | | 472.005041 |
| 199 | 26.97 | 3555.582 | 288436.906 | | 2134.4331 |
| 200 | 26.448 | 3538.979 | 67951.078 | | 502.837977 |
| 201 | 26.967 | 3533.677 | 606124.563 | | 4485.32177 |
| 202 | 27.421 | 3531.154 | 21793.037 | | 161.268474 |
| 203 | 24.129 | 3530.806 | 68264.656 | | 505.158454 |
| 204 | 27.416 | 3524.921 | 101030.172 | | 747.623273 |
| 205 | 26.508 | 3520.84 | 46995.484 | | 347.766582 |
| 206 | 27.773 | 3518.717 | 37848.461 | | 280.078611 |
| 207 | 26.936 | 3517.813 | 749714.938 | | 5547.89054 |
| 208 | 34.02 | 3515.52 | 7304.833 | | 54.0557642 |
| 209 | 27.065 | 3511.743 | 412161.094 | | 3049.9921 |
| 210 | 27.442 | 3509.717 | 25078.984 | | 185.584482 |
| 211 | 21.028 | 3504.793 | 29887.633 | | 221.168484 |
| 212 | 19.053 | 3501.893 | 21114.822 | | 156.249683 |
| 213 | 27.027 | 3495.682 | 2833316.75 | | 20966.544 |
| 214 | 27.411 | 3493.91 | 98902.625 | | 731.879425 |
| 215 | 27.681 | 3479.778 | 17606.455 | | 130.287767 |
| 216 | 26.998 | 3479.73 | 2020814.75 | | 14954.0292 |
| 217 | 27.076 | 3473.736 | 673718 | | 4985.5132 |
| 218 | 27.432 | 3471.466 | 127158.477 | | 940.97273 |
| 219 | 19.129 | 3462.563 | 197973.266 | | 1465.00217 |
| 220 | 22.439 | 3461.026 | 35283.965 | | 261.101341 |
| 221 | 27.678 | 3457.877 | 9517.13 | | 70.426762 |
| 222 | 27.023 | 3457.775 | 3209066 | | 23747.0884 |
| 223 | 31.081 | 3456.538 | 54283.066 | | 401.694688 |
| 224 | 27.408 | 3455.346 | 234864.938 | | 1738.00054 |
| 225 | 21.428 | 3449.671 | 27877.854 | | 206.29612 |
| 226 | 26.975 | 3441.772 | 1378114.63 | 7943.47051 | 10198.0482 |
| 227 | 23.78 | 3439.69 | 46195.805 | | 341.848957 |
| 228 | 33.995 | 3438.31 | 29525.637 | | 218.489714 |
| 229 | 27.307 | 3437.3 | 69127.938 | | 511.546741 |
| 230 | 27.418 | 3433.157 | 184062.516 | | 1362.06262 |
| 231 | 25.03 | 3426.535 | 31401.645 | | 232.372173 |
| 232 | 26.945 | 3425.419 | 241693.109 | | 1788.52901 |
| 233 | 39.51 | 3422.974 | 19537.455 | | 144.577167 |
| 234 | 21.122 | 3422.818 | 189054.766 | | 1399.00527 |
| 235 | 23.945 | 3422.32 | 323563.813 | | 2394.37222 |
| 236 | 27.356 | 3416.57 | 286254.313 | | 2118.28192 |
| 237 | 27.752 | 3409.818 | 70792.961 | | 523.867911 |
| 238 | 23.959 | 3404.934 | 516072.625 | | 3818.93743 |
| 239 | 23.771 | 3402.278 | 118509.031 | | 876.966829 |
| 240 | 22.537 | 3401.814 | 94982.938 | | 702.873741 |
| 241 | 27.278 | 3397.817 | 332921.625 | | 2463.62003 |
| 242 | 32.149 | 3390.576 | 31476.922 | | 232.929223 |
| 243 | 23.818 | 3385.656 | 511551.25 | | 3785.47925 |
| 244 | 26.67 | 3385.149 | 22069.303 | | 163.312842 |
| 245 | 30.445 | 3379.519 | 15737.849 | | 116.460083 |
| 246 | 47.154 | 3378.513 | 38705.031 | | 286.417229 |
| 247 | 32.345 | 3374.638 | 87858.953 | | 650.156252 |
| 248 | 22.155 | 3374.546 | 23420.533 | | 173.311944 |
| 249 | 32.06 | 3368.627 | 127597.813 | | 944.223816 |
| 250 | 26.668 | 3363.626 | 74247.625 | | 549.432425 |
| 251 | 27.298 | 3359.786 | 207612.594 | | 1536.3332 |
| 252 | 30.446 | 3357.522 | 68808.484 | | 509.182782 |
| 253 | 32.051 | 3352.655 | 181481.938 | | 1342.96634 |
| 254 | 19.112 | 3348.489 | 28785.637 | | 213.013714 |
| 255 | 26.668 | 3347.627 | 69073.266 | | 511.142168 |
| 256 | 30.435 | 3341.531 | 95088.156 | | 703.652354 |
| 257 | 21.994 | 3341.505 | 21804.309 | | 161.351887 |
| 258 | 47.117 | 3340.88 | 42485.211 | | 314.390561 |
| 259 | 22.583 | 3338.746 | 84856.977 | | 627.94163 |
| 260 | 22.108 | 3337.336 | 52467.945 | | 388.262793 |
| 261 | 19.069 | 3332.833 | 143378.078 | | 1060.99778 |
| 262 | 32.236 | 3330.681 | 741472.813 | | 5486.89882 |
| 263 | 19.595 | 3329.771 | 22509.027 | | 166.5668 |
| 264 | 22.011 | 3325.803 | 31233.105 | | 231.124977 |
| 265 | 26.671 | 3325.558 | 349720.469 | | 2587.93147 |
| 266 | 30.442 | 3319.545 | 242232.234 | | 1792.51853 |
| 267 | 47.101 | 3319.288 | 35056.176 | | 259.415702 |
| 268 | 22.112 | 3318.936 | 56471.188 | | 417.886791 |
| 269 | 32.133 | 3314.687 | 255600.891 | | 1891.44659 |
| 270 | 23.088 | 3311.52 | 14913.211 | | 110.357761 |
| 271 | 26.667 | 3308.661 | 233464.578 | | 1727.63788 |
| 272 | 47.562 | 3305.413 | 8993.222 | | 66.5498428 |
| 273 | 22.13 | 3303.866 | 168833.016 | | 1249.36432 |
| 274 | 26.694 | 3303.689 | 52945.629 | | 391.797655 |
| 275 | 30.428 | 3303.55 | 190189.688 | | 1407.40369 |
| 276 | 23.897 | 3302.688 | 21441.1 | | 158.66414 |
| 277 | 47.167 | 3302.666 | 31385.865 | | 232.255401 |
| 278 | 22.207 | 3296.58 | 29308.854 | | 216.88552 |
| 279 | 32.299 | 3292.687 | 1206767.88 | | 8930.08228 |
| 280 | 22.855 | 3290.677 | 116553.609 | | 862.496707 |
| 281 | 22.129 | 3287.533 | 19792.754 | | 146.46638 |
| 282 | 26.675 | 3286.98 | 664343.75 | | 4916.14375 |
| 283 | 30.451 | 3281.458 | 136281.25 | | 1008.48125 |
| 284 | 23.91 | 3280.708 | 71741.43 | | 530.886582 |
| 285 | 22.104 | 3280.6 | 132426.766 | | 979.958068 |
| 286 | 27.844 | 3278.421 | 54118.781 | | 400.478979 |
| 287 | 26.669 | 3271.229 | 201540.094 | | 1491.3967 |
| 288 | 21.895 | 3268.819 | 33018.098 | | 244.333925 |
| 289 | 22.073 | 3266.64 | 409110.469 | 2799.38997 | 3027.41747 |
| 290 | 47.665 | 3266.519 | 3664.112 | | 27.1144288 |
| 291 | 26.645 | 3264.684 | 39645.684 | | 293.378062 |
| 292 | 30.445 | 3264.57 | 33225.082 | | 245.865607 |
| 293 | 32.145 | 3261.544 | 154194.859 | | 1141.04196 |
| 294 | 33.87 | 3260.577 | 9901.048 | | 73.2677552 |
| 295 | 22.173 | 3258.623 | 204077.922 | | 1510.17662 |
| 296 | 27.787 | 3256.555 | 242475 | | 1794.315 |
| 297 | 19.366 | 3253.774 | 9140 | | 67.636 |
| 298 | 25.228 | 3252.451 | 40806.734 | | 301.969832 |
| 299 | 26.638 | 3248.195 | 345009.313 | 1768.79215 | 2553.06892 |
| 300 | 21.996 | 3247.551 | 279443.219 | | 2067.87982 |
| 301 | 30.051 | 3238.459 | 73868.313 | | 546.625516 |
| 302 | 21.997 | 3231.53 | 106226.703 | | 786.077602 |
| 303 | 21.23 | 3223.641 | 53736.922 | | 397.653223 |
| 304 | 32.12 | 3222.565 | 133680.984 | | 989.239282 |
| 305 | 26.44 | 3218.542 | 24706.822 | | 182.830483 |
| 306 | 22.018 | 3209.292 | 976666.75 | | 7227.33395 |
| 307 | 19.501 | 3205.379 | 172521.531 | | 1276.65933 |
| 308 | 25.911 | 3200.625 | 30798.475 | | 227.908715 |
| 309 | 30.054 | 3200.553 | 4472.367 | | 329.095516 |
| 310 | 26.275 | 3197.269 | 120978.656 | | 895.242054 |
| 311 | 18.965 | 3195.145 | 1365.552 | | 10.1050848 |
| 312 | 21.98 | 3193.339 | 46203.789 | | 341.908039 |
| 313 | 47.564 | 3190.567 | 6346.223 | | 46.9620502 |
| 314 | 26.894 | 3187.444 | 70904.891 | | 524.696193 |
| 315 | 47.302 | 3187.001 | 26800.559 | | 198.324137 |
| 316 | 31.823 | 3183.572 | 62859.313 | | 465.158916 |
| 317 | 27.686 | 3181.527 | 130513.648 | | 965.800995 |
| 318 | 20.726 | 3180.29 | 149180.203 | | 1103.9335 |
| 319 | 26.411 | 3175.418 | 95767.344 | | 708.678346 |
| 320 | 26.942 | 3166.985 | 32730.686 | | 242.207076 |
| 321 | 21.419 | 3166.416 | 143203.063 | | 1059.70267 |
| 322 | 26.351 | 3159.36 | 255623.938 | | 1891.61714 |
| 323 | 29.385 | 3158.431 | 24115.967 | | 178.458156 |
| 324 | 20.733 | 3153.308 | 214732.906 | | 1589.0235 |
| 325 | 30.597 | 3152.559 | 13341.057 | | 98.7238218 |
| 326 | 23.226 | 3152.484 | 36119.008 | | 267.280659 |
| 327 | 26.922 | 3149.624 | 114393.813 | | 846.514216 |
| 328 | 47.176 | 3148.686 | 135798.828 | | 1004.91133 |
| 329 | 22.866 | 3148.426 | 81875.969 | | 605.882171 |
| 330 | 32.019 | 3145.601 | 228017.438 | | 1687.32904 |
| 331 | 27.665 | 3143.604 | 316068.156 | | 2338.90435 |
| 332 | 26.033 | 3139.523 | 182642.438 | | 1351.55404 |
| 333 | 60.141 | 3138.904 | 21558.727 | | 159.53458 |
| 334 | 26.459 | 3137.595 | 153225.172 | | 1133.86627 |
| 335 | 29.41 | 3137.466 | 52329.82 | | 387.240668 |
| 336 | 26.846 | 3130.813 | 68584.234 | | 507.523332 |
| 337 | 30.606 | 3130.447 | 81592.641 | | 603.785543 |
| 338 | 47.079 | 3126.892 | 51823.832 | | 383.496357 |
| 339 | 26.408 | 3121.15 | 278297.125 | | 2059.39873 |
| 340 | 26.149 | 3117.737 | 33942.793 | | 251.176668 |
| 341 | 19.395 | 3116.31 | 11625.266 | | 86.0269684 |
| 342 | 29.428 | 3115.482 | 75318.172 | | 557.354473 |
| 343 | 21.751 | 3111.371 | 83680.453 | | 619.235352 |
| 344 | 26.927 | 3108.555 | 45816.859 | | 339.044757 |
| 345 | 47.062 | 3106.709 | 11864.485 | | 87.797189 |
| 346 | 26.248 | 3101.309 | 144908.688 | | 1072.32429 |
| 347 | 56.788 | 3101.218 | 12576.415 | | 93.065471 |
| 348 | 59.981 | 3101.187 | 25661.803 | | 189.897342 |
| 349 | 29.415 | 3099.508 | 130302.75 | | 964.24035 |
| 350 | 26.467 | 3099.349 | 120340.961 | | 890.523111 |
| 351 | 38.257 | 3093.515 | 133819.891 | | 990.267193 |
| 352 | 26.873 | 3092.646 | 79548.953 | | 588.662252 |
| 353 | 25.409 | 3091.628 | 74034.063 | | 547.852066 |
| 354 | 21.782 | 3089.383 | 140512.359 | | 1039.79146 |
| 355 | 26.165 | 3086.295 | 48012.742 | | 355.294291 |
| 356 | 60.105 | 3085.019 | 7315.923 | | 54.1378302 |
| 357 | 28.833 | 3083.524 | 10016.655 | | 74.123247 |
| 358 | 45.098 | 3080.227 | 54908.891 | | 406.325793 |
| 359 | 26.28 | 3079.63 | 200529.125 | | 1483.91553 |
| 360 | 29.426 | 3077.473 | 365503.375 | | 2704.72498 |
| 361 | 19.423 | 3076.296 | 244458.344 | | 1808.99175 |
| 362 | 21.716 | 3073.391 | 19542.326 | | 144.613212 |
| 363 | 20.524 | 3064.362 | 67399.18 | | 498.753932 |
| 364 | 26.328 | 3063.327 | 269186.313 | | 1991.97872 |
| 365 | 29.434 | 3061.477 | 296489.781 | | 2194.02438 |
| 366 | 23.304 | 3060.546 | 57847.941 | | 428.074763 |
| 367 | 22.449 | 3059.408 | 131458.734 | | 972.794632 |
| 368 | 38.508 | 3055.55 | 361900.688 | | 2678.06509 |
| 369 | 21.798 | 3051.223 | 597188.688 | | 4419.19629 |
| 370 | 26.15 | 3049.373 | 185624.422 | | 1373.62072 |
| 371 | 28.742 | 3045.463 | 33384.316 | | 247.043938 |
| 372 | 22.439 | 3043.271 | 33506.316 | | 247.946738 |
| 373 | 26.25 | 3041.552 | 275291.813 | | 2037.15942 |
| 374 | 29.434 | 3039.565 | 1232005.13 | | 9116.83793 |
| 375 | 31.704 | 3039.504 | 14986.015 | | 110.896511 |
| 376 | 31.401 | 3039.5 | 17652.994 | | 130.632156 |
| 377 | 30.288 | 3038.548 | 53233.535 | | 393.928159 |
| 378 | 21.744 | 3036.442 | 318586.125 | | 2357.53733 |
| 379 | 26.1 | 3033.408 | 63530.199 | | 470.123473 |
| 380 | 29.428 | 3023.508 | 440285.75 | | 3258.11455 |
| 381 | 23.309 | 3023.197 | 35494.395 | | 262.658523 |
| 382 | 20.855 | 3022.938 | 49940.453 | | 369.559352 |
| 383 | 22.458 | 3021.485 | 264509 | | 1957.3666 |
| 384 | 38.552 | 3018.517 | 10132.168 | | 74.9780432 |
| 385 | 47.558 | 3016.717 | 14738.372 | | 109.063953 |
| 386 | 19.317 | 3015.296 | 7210.649 | | 53.3588026 |
| 387 | 21.764 | 3013.407 | 2745916.75 | | 20319.784 |
| 388 | 52.853 | 3013.224 | 6434.849 | | 47.6178826 |
| 389 | 26.165 | 3011.541 | 328700.25 | 1828.86053 | 2432.38185 |
| 390 | 53.981 | 3011.136 | 9101.616 | | 67.3519584 |
| 391 | 22.771 | 3002.372 | 31709.063 | | 234.647066 |
| 392 | 29.689 | 3001.477 | 1807418.13 | | 13374.8941 |
| 393 | 29.788 | 2999.396 | 10075.938 | | 74.5619412 |
| 394 | 21.692 | 2999.39 | 458502.219 | | 3392.91642 |
| 395 | 47.256 | 2994.527 | 99911.469 | | 739.344871 |
| 396 | 54.066 | 2994.525 | 27062.277 | | 200.26085 |
| 397 | 21.71 | 2979.845 | 137706.313 | | 1019.02672 |
| 398 | 47.277 | 2978.338 | 98216.141 | | 726.799443 |
| 399 | 25.825 | 2977.451 | 51783.652 | | 383.199025 |
| 400 | 19.356 | 2977.313 | 141777.906 | | 1049.1565 |
| 401 | 28.834 | 2976.801 | 13806.644 | | 102.169166 |
| 402 | 22.995 | 2973.765 | 71126.297 | | 526.334598 |
| 403 | 54.065 | 2973.1 | 72137.359 | | 533.816457 |
| 404 | 23.115 | 2961.255 | 24611.352 | | 182.124005 |
| 405 | 29.606 | 2959.461 | 93377.992 | | 690.997141 |
| 406 | 54.087 | 2956.985 | 34407.879 | | 254.618305 |
| 407 | 47.177 | 2956.613 | 146180.453 | | 1081.73535 |
| 408 | 47.261 | 2939.827 | 19831.465 | | 146.752841 |
| 409 | 28.882 | 2939.32 | 8665.473 | | 64.1245002 |
| 410 | 23.159 | 2939.275 | 60206.324 | | 445.526798 |
| 411 | 47.074 | 2935.554 | 119347.43 | | 883.170982 |
| 412 | 54.081 | 2935.165 | 71573.266 | | 529.642168 |
| 413 | 29.202 | 2930.452 | 119639.5 | | 885.3323 |
| 414 | 23.112 | 2923.278 | 70444.055 | | 521.286007 |
| 415 | 47.154 | 2918.618 | 78469.992 | | 580.677941 |
| 416 | 25.694 | 2917.375 | 149087.75 | | 1103.24935 |
| 417 | 47.052 | 2914.597 | 30523.621 | | 225.874795 |
| 418 | 29.136 | 2914.306 | 40968.273 | | 303.16522 |
| 419 | 29.197 | 2913.596 | 18412.203 | | 136.250302 |
| 420 | 19.329 | 2913.459 | 99422.539 | | 735.726789 |
| 421 | 23.807 | 2912.353 | 384302.219 | | 2843.83642 |
| 422 | 23.119 | 2901.3 | 312914.094 | | 2315.5643 |
| 423 | 25.911 | 2898.456 | 18701.76 | | 138.393024 |
| 424 | 38.603 | 2898.411 | 5865.952 | | 43.4080448 |
| 425 | 29.2 | 2892.487 | 466333.719 | | 3450.86952 |
| 426 | 19.686 | 2887.621 | 72285.609 | | 534.913507 |
| 427 | 23.124 | 2885.349 | 248764.953 | | 1840.86065 |
| 428 | 23.181 | 2879.347 | 44899.32 | | 332.254968 |
| 429 | 29.188 | 2876.501 | 149361.734 | | 1105.27683 |
| 430 | 20.174 | 2864.504 | 170329.094 | | 1260.4353 |
| 431 | 23.108 | 2863.359 | 1062454 | | 7862.1596 |
| 432 | 25.816 | 2861.674 | 45627.336 | | 337.642286 |
| 433 | 29.189 | 2854.531 | 504398.031 | | 3732.54543 |
| 434 | 22.467 | 2849.122 | 98682.781 | | 730.252579 |
| 435 | 23.101 | 2847.369 | 329935.469 | | 2441.52247 |
| 436 | 30.997 | 2840.951 | 29558.09 | | 218.729866 |
| 437 | 23.206 | 2840.788 | 288407.906 | | 2134.2185 |
| 438 | 45.501 | 2839.222 | 19862.145 | | 146.979873 |
| 439 | 22.887 | 2838.17 | 401500.781 | | 2971.10578 |
| 440 | 23.479 | 2837.179 | 47790.191 | | 353.647413 |
| 441 | 23.12 | 2825.369 | 2478449 | 13220.0253 | 18340.5226 |
| 442 | 47.398 | 2824.479 | 66262.844 | | 490.345046 |
| 443 | 25.837 | 2823.447 | 161446.953 | | 1194.70745 |
| 444 | 23.493 | 2823.263 | 13751.687 | | 101.762484 |
| 445 | 31.001 | 2818.979 | 87299.125 | | 646.013525 |
| 446 | 25.738 | 2818.354 | 25736.781 | | 190.452179 |
| 447 | 45.063 | 2816.103 | 114974.227 | | 850.80928 |
| 448 | 23.099 | 2808.413 | 98189.281 | | 726.600679 |
| 449 | 47.299 | 2802.496 | 96102.875 | | 711.161275 |
| 450 | 25.713 | 2802.334 | 14592.676 | | 107.985802 |
| 451 | 34.356 | 2802.334 | 85762.539 | | 634.642789 |
| 452 | 23.512 | 2799.188 | 1044906.38 | | 7732.30718 |
| 453 | 47.289 | 2786.471 | 144146.313 | | 1066.68272 |
| 454 | 29.59 | 2781.738 | 10014.258 | | 74.1055092 |
| 455 | 25.737 | 2780.371 | 61741.945 | | 456.890393 |
| 456 | 56.764 | 2772.108 | 7783.357 | | 57.5968418 |
| 457 | 54.717 | 2772.02 | 10717.143 | | 79.3068582 |
| 458 | 21.387 | 2766.811 | 49527.543 | | 366.503818 |
| 459 | 47.176 | 2764.54 | 301319.875 | | 2229.76708 |
| 460 | 34.412 | 2764.419 | 401417.969 | | 2970.49297 |
| 461 | 22.615 | 2755.244 | 63484.922 | | 469.788423 |
| 462 | 26.046 | 2754.507 | 67685.281 | | 500.871079 |
| 463 | 45.398 | 2751.163 | 21037.008 | | 155.673859 |
| 464 | 47.208 | 2747.715 | 118178.953 | | 874.524252 |
| 465 | 47.1 | 2743.596 | 170694.891 | | 1263.14219 |
| 466 | 25.731 | 2742.398 | 138049.344 | 1296.81025 | 1021.56515 |
| 467 | 19.271 | 2733.962 | 31792.85 | | 235.26709 |
| 468 | 22.636 | 2733.228 | 122354.352 | | 905.422205 |
| 469 | 47.344 | 2726.574 | 36973.457 | | 273.603582 |
| 470 | 34.503 | 2726.404 | 137639.047 | | 1018.52895 |
| 471 | 21.913 | 2720.199 | 13717.608 | | 101.510299 |
| 472 | 19.219 | 2718.289 | 12883.684 | | 95.3392616 |
| 473 | 19.051 | 2717.641 | 33752.184 | | 249.766162 |
| 474 | 22.626 | 2717.287 | 228040.188 | | 1687.49739 |
| 475 | 34.147 | 2706.346 | 28067.836 | | 207.701986 |
| 476 | 22.609 | 2700.793 | 87403.336 | | 646.784686 |
| 477 | 19.26 | 2696.284 | 111610.914 | | 825.920764 |
| 478 | 22.644 | 2695.322 | 489907.813 | 3209.57073 | 3625.31782 |
| 479 | 29.391 | 2688.188 | 62864.418 | | 465.196693 |
| 480 | 53.432 | 2685.952 | 9794.151 | | 72.4767174 |
| 481 | 21.026 | 2682.281 | 54992.879 | | 406.947305 |
| 482 | 21.953 | 2682.264 | 164811.766 | | 1219.60707 |
| 483 | 19.234 | 2680.347 | 52036.555 | | 385.070507 |
| 484 | 22.642 | 2679.325 | 348845.031 | 2843.92567 | 2581.45323 |
| 485 | 53.436 | 2669.979 | 28587.529 | | 211.547715 |
| 486 | 29.453 | 2666.015 | 134377.281 | | 994.391879 |
| 487 | 21.154 | 2663.759 | 77780.211 | | 575.573561 |
| 488 | 22.618 | 2663.307 | 45806.359 | | 338.967057 |
| 489 | 45.5 | 2663.131 | 10745.179 | | 79.5143246 |
| 490 | 45.202 | 2663.093 | 9994.085 | | 73.956229 |
| 491 | 19.308 | 2658.394 | 889974 | | 6585.8076 |
| 492 | 22.792 | 2653.835 | 10012.66 | | 74.093684 |
| 493 | 29.409 | 2649.748 | 209835.844 | | 1552.78525 |
| 494 | 53.492 | 2647.894 | 109457.719 | | 809.987121 |
| 495 | 34.123 | 2646.285 | 83952.914 | | 621.251564 |
| 496 | 21.014 | 2644.389 | 143070.844 | | 1058.72425 |
| 497 | 24.958 | 2641.325 | 9244.972 | | 68.4127928 |
| 498 | 22.26 | 2640.197 | 37788.34 | | 279.633716 |
| 499 | 28.958 | 2638.48 | 18692.865 | | 138.327201 |
| 500 | 25.376 | 2637.34 | 43694.086 | | 323.336236 |
| 501 | 47.401 | 2632.529 | 85583.328 | | 633.316627 |
| 502 | 53.456 | 2631.985 | 46383.336 | | 343.236686 |
| 503 | 23.975 | 2631.124 | 19223.193 | | 142.251628 |
| 504 | 34.021 | 2630.294 | 11402.769 | | 84.3804906 |
| 505 | 29.459 | 2628.371 | 42183.633 | | 312.158884 |
| 506 | 22.281 | 2624.209 | 24509.521 | | 181.370455 |
| 507 | 29.621 | 2622.336 | 14705.738 | | 108.822461 |
| 508 | 25.361 | 2620.846 | 14378.705 | | 106.402417 |
| 509 | 25.496 | 2620.25 | 48204.504 | | 356.71333 |
| 510 | 22.285 | 2618.226 | 25010.121 | | 185.074895 |
| 511 | 47.465 | 2615.493 | 7661.221 | | 56.6930354 |
| 512 | 47.164 | 2615.485 | 6246.06 | | 46.220844 |
| 513 | 29.331 | 2611.332 | 51577.367 | | 381.672516 |
| 514 | 47.268 | 2610.486 | 133230.797 | | 985.907898 |
| 515 | 53.423 | 2609.623 | 65116.852 | | 481.864705 |
| 516 | 34.267 | 2608.302 | 477189.5 | | 3531.2023 |
| 517 | 36.436 | 2608.217 | 49190.086 | | 364.006636 |
| 518 | 25.258 | 2606.235 | 8864.643 | | 65.5983582 |
| 519 | 22.254 | 2602.233 | 250098.953 | | 1850.73225 |
| 520 | 28.955 | 2600.859 | 42927.066 | | 317.660288 |
| 521 | 47.321 | 2594.475 | 174972.719 | | 1294.79812 |
| 522 | 23.962 | 2593.309 | 31956.934 | | 236.481312 |
| 523 | 34.191 | 2592.376 | 73723.086 | | 545.550836 |
| 524 | 29.194 | 2589.416 | 24432.801 | | 180.802727 |
| 525 | 21.055 | 2586.81 | 47437.063 | | 3311.03427 |
| 526 | 29.643 | 2584.386 | 51728.656 | | 382.792054 |
| 527 | 25.285 | 2583.339 | 57463.207 | | 425.227732 |
| 528 | 22.289 | 2580.278 | 184628.453 | | 1366.25055 |
| 529 | 25.458 | 2579.266 | 17502.498 | | 129.518485 |
| 530 | 23.288 | 2577.366 | 55801.965 | | 412.934541 |
| 531 | 45.349 | 2575.055 | 55573.074 | | 411.240748 |
| 532 | 47.193 | 2572.49 | 283804.656 | | 2100.15445 |
| 533 | 34.366 | 2570.327 | 278517.688 | | 2061.03089 |
| 534 | 53.788 | 2565.094 | 15309.151 | | 113.287717 |
| 535 | 22.285 | 2564.171 | 522868.969 | 5032.24845 | 3869.23037 |
| 536 | 25.111 | 2563.298 | 69871.031 | | 517.045629 |
| 537 | 21.035 | 2563.271 | 214366.938 | | 1586.31534 |
| 538 | 28.935 | 2560.247 | 22648.59 | | 167.599566 |
| 539 | 19.241 | 2559.267 | 287009.563 | | 2123.87077 |
| 540 | 47.221 | 2555.777 | 175942.031 | | 1301.97103 |
| 541 | 23.992 | 2554.181 | 34969.023 | | 258.77077 |
| 542 | 47.112 | 2551.558 | 249344.906 | | 1845.1523 |
| 543 | 54.133 | 2549.837 | 10977.327 | | 81.2322198 |
| 544 | 53.85 | 2548.39 | 47193.648 | | 349.232995 |
| 545 | 21.105 | 2548.131 | 191514.047 | | 1417.20395 |
| 546 | 25.467 | 2541.55 | 46554.676 | | 344.504602 |
| 547 | 22.079 | 2540.416 | 70642.961 | | 522.757911 |
| 548 | 47.061 | 2539.548 | 9106.932 | | 67.3912968 |
| 549 | 22.976 | 2538.006 | 43224.359 | | 319.860257 |
| 550 | 23.949 | 2536.124 | 11100.246 | | 82.1418204 |
| 551 | 47.048 | 2534.558 | 93213.172 | | 689.777473 |
| 552 | 53.76 | 2531.854 | 6839.3 | | 50.61082 |
| 553 | 47.021 | 2530.606 | 81025.352 | | 599.587605 |
| 554 | 20.664 | 2527.292 | 182897.938 | | 1353.44474 |
| 555 | 53.86 | 2525.836 | 124043.008 | | 917.918259 |
| 556 | 25.163 | 2525.357 | 195437.094 | | 1446.2345 |
| 557 | 24.934 | 2521.218 | 35385.203 | | 261.850502 |
| 558 | 49.205 | 2520.943 | 7070.417 | | 52.3210858 |
| 559 | 22.186 | 2518.416 | 12192.377 | | 90.2235898 |
| 560 | 23.995 | 2514.154 | 31468.1 | | 232.86394 |
| 561 | 53.893 | 2509.854 | 74708.906 | | 552.845904 |
| 562 | 53.849 | 2503.849 | 66345.75 | | 490.95855 |
| 563 | 22.127 | 2502.45 | 883793.5 | | 6540.0719 |
| 564 | 23.949 | 2498.133 | 13605.738 | | 100.682461 |
| 565 | 30.369 | 2491.037 | 55242.645 | | 408.795573 |
| 566 | 53.921 | 2487.847 | 73487.078 | | 543.804377 |
| 567 | 45.471 | 2487.024 | 20252.117 | | 149.865666 |
| 568 | 53.508 | 2483.887 | 9788.317 | | 72.4335458 |
| 569 | 55.095 | 2483.245 | 22470.289 | | 166.280139 |
| 570 | 24.966 | 2483.135 | 101523.984 | | 751.277482 |
| 571 | 50.133 | 2480.983 | 6654.337 | | 49.2420938 |
| 572 | 28.907 | 2480.171 | 48322.785 | | 357.588609 |
| 573 | 37.468 | 2479.164 | 10220.078 | | 75.6285772 |
| 574 | 29.264 | 2471.298 | 17940.619 | | 132.760581 |
| 575 | 25.284 | 2468.22 | 27459.982 | | 203.203867 |
| 576 | 53.575 | 2466.851 | 50476.148 | | 373.523495 |
| 577 | 55.082 | 2466.643 | 85574.617 | | 633.252166 |
| 578 | 28.208 | 2466.149 | 19103.148 | | 141.363295 |
| 579 | 30.326 | 2452.741 | 105720.547 | | 782.332048 |
| 580 | 25.383 | 2452.241 | 25429.555 | | 188.178707 |
| 581 | 53.558 | 2444.922 | 132386.922 | | 979.663223 |
| 582 | 55.038 | 2444.863 | 166593.141 | | 1232.78924 |
| 583 | 54.419 | 2443.898 | 14516.007 | | 107.418452 |
| 584 | 28.896 | 2442.177 | 99161.281 | | 733.793479 |
| 585 | 47.436 | 2440.325 | 82396.445 | | 609.733693 |
| 586 | 22.331 | 2439.349 | 26883.18 | | 198.935532 |
| 587 | 20.725 | 2437.156 | 67598.68 | | 500.230232 |
| 588 | 30.367 | 2430.738 | 112506.719 | | 832.549721 |
| 589 | 25.309 | 2430.18 | 9069.742 | | 67.1160908 |
| 590 | 37.568 | 2430.001 | 10098.263 | | 74.7271462 |
| 591 | 55.058 | 2428.844 | 151840.578 | | 1123.62028 |
| 592 | 53.565 | 2428.839 | 97552.461 | | 721.888211 |
| 593 | 21.874 | 2427.305 | 277859.406 | | 2056.1596 |
| 594 | 28.784 | 2422.657 | 49308.391 | | 364.882093 |
| 595 | 47.264 | 2418.405 | 102784.953 | | 760.608652 |
| 596 | 28.611 | 2418.047 | 23545.922 | | 174.239823 |
| 597 | 30.328 | 2415.783 | 100262.688 | | 741.943891 |
| 598 | 20.7 | 2415.168 | 241525.406 | | 1787.288 |
| 599 | 28.755 | 2415.065 | 27699.828 | | 204.978727 |
| 600 | 19.242 | 2414.29 | 19697.506 | | 145.761544 |
| 601 | 55.019 | 2411.833 | 8743.657 | | 64.7030618 |
| 602 | 37.119 | 2408.022 | 95515.813 | | 706.817016 |
| 603 | 24.956 | 2407.143 | 19052.658 | | 140.989669 |
| 604 | 56.103 | 2406.969 | 2105.086 | | 15.5776364 |
| 605 | 53.549 | 2406.949 | 105913 | | 783.7562 |
| 606 | 55.077 | 2406.902 | 128330.906 | | 949.648704 |
| 607 | 21.91 | 2405.312 | 22780.617 | | 168.576566 |
| 608 | 20.28 | 2404.141 | 42368.875 | | 313.529675 |
| 609 | 47.296 | 2402.426 | 168612.344 | | 1247.73135 |
| 610 | 20.557 | 2401.462 | 109291.289 | | 808.755539 |
| 611 | 28.781 | 2398.085 | 20548.543 | | 152.059218 |
| 612 | 28.626 | 2396.215 | 186626 | | 1381.0324 |
| 613 | 54.355 | 2395.78 | 17539.246 | | 129.79042 |
| 614 | 19.791 | 2395.33 | 38127.117 | | 282.140666 |
| 615 | 53.832 | 2395.108 | 25210.896 | | 186.56063 |
| 616 | 22.181 | 2393.58 | 28325.973 | | 209.6122 |
| 617 | 37.389 | 2392.069 | 32789.227 | | 242.64028 |
| 618 | 36.842 | 2392.022 | 16480.713 | | 121.957276 |
| 619 | 53.509 | 2390.96 | 7808.705 | | 57.784417 |
| 620 | 55.017 | 2390.746 | 56777.559 | | 420.153937 |
| 621 | 21.858 | 2389.367 | 1415681.5 | | 10476.0431 |
| 622 | 28.783 | 2384.878 | 67750.172 | | 501.351273 |
| 623 | 54.213 | 2381.804 | 9437.86 | | 69.840164 |
| 624 | 53.96 | 2379.819 | 13638.621 | | 100.925795 |
| 625 | 28.586 | 2379.768 | 179069.688 | | 1325.11569 |
| 626 | 20.738 | 2377.238 | 713037.375 | | 5276.47658 |
| 627 | 28.792 | 2377.105 | 243979.281 | | 1805.44668 |
| 628 | 54.034 | 2374.369 | 98473.961 | | 728.707311 |
| 629 | 38.119 | 2370.071 | 587311.313 | | 4346.10372 |
| 630 | 55.054 | 2368.829 | 45047.953 | | 333.354852 |
| 631 | 49.833 | 2368.79 | 7574.214 | | 56.0491836 |
| 632 | 21.712 | 2364.035 | 14890.036 | | 110.186266 |
| 633 | 47.251 | 2363.877 | 169502.969 | | 1254.32197 |
| 634 | 47.112 | 2359.46 | 226706.625 | | 1677.62903 |
| 635 | 54.06 | 2358.796 | 271180 | | 2006.732 |
| 636 | 28.638 | 2358.069 | 484792 | | 3587.4608 |
| 637 | 30.392 | 2356.841 | 24718.348 | | 182.915775 |
| 638 | 49.349 | 2356.439 | 49754.129 | | 368.180555 |
| 639 | 22.229 | 2355.161 | 64185.695 | | 474.974143 |
| 640 | 37.22 | 2354.086 | 71290.633 | | 527.550684 |
| 641 | 54.995 | 2351.738 | 14965.655 | | 110.745847 |
| 642 | 19.26 | 2343.309 | 31234.203 | | 231.133102 |
| 643 | 54.345 | 2342.704 | 55254.418 | | 408.882693 |
| 644 | 21.753 | 2342.053 | 57971.406 | | 428.988404 |
| 645 | 53.764 | 2341.883 | 102171.742 | | 756.070891 |
| 646 | 28.619 | 2341.757 | 395942.094 | | 2929.9715 |
| 647 | 24.143 | 2341.072 | 185665.328 | | 1373.92343 |
| 648 | 28.832 | 2339.135 | 119528.75 | | 884.51275 |
| 649 | 54.092 | 2336.821 | 849316.625 | | 6284.94303 |
| 650 | 49.582 | 2334.924 | 16711.031 | | 123.661629 |
| 651 | 49.932 | 2332.825 | 7154.382 | | 52.9424268 |
| 652 | 38.111 | 2332.118 | 724905.563 | | 5364.30117 |
| 653 | 28.429 | 2332.011 | 26272.477 | | 194.41633 |
| 654 | 49.784 | 2330.848 | 18374.775 | | 135.973335 |
| 655 | 20.777 | 2329.976 | 13845.191 | | 102.454413 |
| 656 | 20.781 | 2328.12 | 114579.438 | | 847.887841 |
| 657 | 21.695 | 2326.051 | 33955.465 | | 251.270441 |
| 658 | 28.514 | 2325.829 | 27821.584 | | 205.879722 |
| 659 | 24.109 | 2325.039 | 347883.406 | | 2574.3372 |
| 660 | 18.808 | 2323.646 | 3501.718 | | 25.9127132 |
| 661 | 21.857 | 2322.105 | 27377.75 | | 202.59535 |
| 662 | 54.036 | 2320.85 | 1445477 | | 10696.5298 |
| 663 | 21.736 | 2320.083 | 17368.453 | | 128.526552 |
| 664 | 28.756 | 2320.083 | 1394689.88 | | 10320.7051 |
| 665 | 31.151 | 2319.995 | 18706.303 | | 138.426642 |
| 666 | 50.101 | 2319.65 | 21160.838 | | 156.590201 |
| 667 | 49.596 | 2318.781 | 65589.969 | | 485.365771 |
| 668 | 53.96 | 2314.817 | 5407.608 | | 40.0162992 |
| 669 | 28.434 | 2313.129 | 21076.289 | | 155.964539 |
| 670 | 20.695 | 2312.394 | 183844.266 | | 1360.44757 |
| 671 | 45.447 | 2310.83 | 8350.151 | | 61.7911174 |
| 672 | 28.377 | 2309.901 | 32903.531 | | 243.486129 |
| 673 | 24.091 | 2309.052 | 53750.734 | | 397.755432 |
| 674 | 28.352 | 2308.815 | 65167.617 | | 482.240366 |
| 675 | 19.413 | 2306.093 | 11910.861 | | 88.1403714 |
| 676 | 47.374 | 2305.43 | 20267.41 | | 149.978834 |
| 677 | 30.479 | 2304.938 | 64574.715 | | 477.852891 |
| 678 | 54.005 | 2304.69 | 601818.875 | | 4453.45968 |
| 679 | 55.551 | 2304.254 | 20926.719 | | 154.857721 |
| 680 | 28.66 | 2304.2 | 441698.625 | | 3268.56983 |
| 681 | 21.748 | 2304.056 | 491680.813 | | 3638.43802 |
| 682 | 54.026 | 2298.86 | 2803671 | | 20747.1654 |
| 683 | 20.539 | 2298.221 | 189552.234 | | 1402.68653 |
| 684 | 30.798 | 2295.988 | 10958.861 | | 81.0955714 |
| 685 | 50.105 | 2295.884 | 10295.519 | | 76.1868406 |
| 686 | 19.179 | 2295.451 | 24928.195 | | 184.468643 |
| 687 | 28.816 | 2295.237 | 260138.016 | | 1925.02132 |
| 688 | 28.385 | 2295.094 | 55681.145 | | 412.040473 |
| 689 | 21.739 | 2288.101 | 57605.777 | | 426.28275 |
| 690 | 24.096 | 2287.057 | 1262872.75 | | 9345.25835 |
| 691 | 28.388 | 2287.048 | 297568.75 | | 2202.00875 |
| 692 | 20.5 | 2286.111 | 21349.635 | | 157.987299 |
| 693 | 24.027 | 2283.095 | 24350.102 | | 180.190755 |
| 694 | 54.068 | 2282.898 | 2728088.75 | | 20187.8568 |
| 695 | 28.732 | 2282.105 | 667956.938 | 6281.39651 | 4942.88134 |
| 696 | 21.782 | 2282.103 | 58645.039 | | 433.973289 |
| 697 | 30.387 | 2282 | 115145.461 | | 852.076411 |
| 698 | 38.199 | 2281.901 | 31642.785 | | 234.156609 |
| 699 | 38.703 | 2281.894 | 8948.516 | | 66.2190184 |
| 700 | 40.729 | 2281.884 | 69357.18 | | 513.243132 |
| 701 | 37.847 | 2281.877 | 7632.981 | | 56.4840594 |
| 702 | 49.628 | 2280.479 | 11230.125 | | 83.102925 |
| 703 | 53.207 | 2278.702 | 26455.098 | | 195.767725 |
| 704 | 28.903 | 2273.115 | 293589.719 | | 2172.56392 |
| 705 | 28.157 | 2272.659 | 27751.025 | | 205.357585 |
| 706 | 28.368 | 2271.091 | 240296.203 | | 1778.1919 |
| 707 | 23.951 | 2271.069 | 46381.387 | | 343.222264 |
| 708 | 54.563 | 2266.722 | 53080.328 | | 392.794427 |
| 709 | 53.746 | 2266.569 | 194273.734 | | 1437.62563 |
| 710 | 21.735 | 2266.113 | 834853.813 | | 6177.91822 |
| 711 | 47.142 | 2264.341 | 11589.805 | | 85.764557 |
| 712 | 25.004 | 2264.038 | 10342.722 | | 76.5361428 |
| 713 | 54.111 | 2260.906 | 3409623 | | 25231.2102 |
| 714 | 39.005 | 2259.881 | 8171.715 | | 60.470691 |
| 715 | 38.018 | 2259.88 | 89646.383 | | 663.383234 |
| 716 | 29.265 | 2257.021 | 369411.719 | | 2733.64672 |
| 717 | 28.144 | 2250.717 | 73907.656 | | 546.916654 |
| 718 | 28.424 | 2249.076 | 762669.688 | | 5643.75569 |
| 719 | 20.437 | 2249.042 | 143049.578 | | 1058.56688 |
| 720 | 47.446 | 2248.591 | 114672.375 | | 848.575575 |
| 721 | 53.175 | 2246.379 | 18363.697 | | 135.891358 |
| 722 | 54.105 | 2244.921 | 1221368.75 | | 9038.12875 |
| 723 | 53.209 | 2240.749 | 57652.742 | | 426.630291 |
| 724 | 49.832 | 2237.759 | 5953.025 | | 44.052385 |
| 725 | 28.924 | 2235.175 | 214885.766 | | 1590.15467 |
| 726 | 28.13 | 2234.935 | 156541.781 | | 1158.40918 |
| 727 | 28.391 | 2233.099 | 305448.406 | | 2260.3182 |
| 728 | 20.474 | 2232.684 | 41022.949 | | 303.569823 |
| 729 | 25.029 | 2231.969 | 11965.051 | | 88.5413774 |
| 730 | 23.853 | 2230.051 | 38128.531 | | 282.151129 |
| 731 | 25.127 | 2227.077 | 31312.469 | | 231.712271 |
| 732 | 47.253 | 2226.358 | 138834.063 | | 1027.37207 |
| 733 | 24.982 | 2226.04 | 37025.727 | | 273.99038 |
| 734 | 54.067 | 2222.955 | 1451186.5 | | 10738.7801 |
| 735 | 55.587 | 2222.909 | 18411.467 | | 136.244856 |
| 736 | 39.072 | 2221.946 | 758603.125 | | 5613.66313 |
| 737 | 33.365 | 2221.747 | 13675.914 | | 101.201764 |
| 738 | 49.924 | 2221.487 | 22766.725 | | 168.473765 |
| 739 | 28.601 | 2216.12 | 62841.969 | | 465.030571 |
| 740 | 21.813 | 2215.931 | 38498.379 | | 284.888005 |
| 741 | 28.165 | 2212.947 | 407865.344 | | 3018.20355 |
| 742 | 20.541 | 2211.332 | 175885.453 | | 1301.55235 |
| 743 | 28.425 | 2211.08 | 604871.438 | | 4476.04864 |
| 744 | 47.358 | 2210.38 | 198223.844 | | 1466.85645 |
| 745 | 24.99 | 2209.092 | 148361.516 | | 1097.87522 |
| 746 | 27.351 | 2208.986 | 43820.238 | | 324.269761 |
| 747 | 34.776 | 2208.878 | 7891.04 | | 58.393696 |
| 748 | 23.983 | 2208.045 | 1445503.75 | | 10696.7278 |
| 749 | 33.037 | 2207.824 | 20242.492 | | 149.794441 |
| 750 | 53.207 | 2207.742 | 5340.262 | | 39.5179388 |
| 751 | 29.961 | 2206.693 | 69426.469 | | 513.755871 |
| 752 | 39.013 | 2205.963 | 658740.688 | | 4874.68109 |
| 753 | 54.008 | 2203.897 | 11335.302 | | 83.8812348 |
| 754 | 53.189 | 2202.779 | 65413.605 | | 484.060677 |
| 755 | 19.293 | 2200.036 | 22179.658 | | 164.129469 |
| 756 | 55.068 | 2199.737 | 7793.02 | | 57.668348 |
| 757 | 28.127 | 2197.011 | 487901.156 | | 3610.46855 |
| 758 | 20.181 | 2194.916 | 131013.984 | | 969.503482 |
| 759 | 50.327 | 2194.728 | 41764.328 | | 309.056027 |
| 760 | 24.981 | 2193.839 | 73573.773 | | 544.44592 |
| 761 | 20.446 | 2193.108 | 20270.781 | | 150.003779 |
| 762 | 54.947 | 2192.113 | 67683.648 | | 500.858995 |
| 763 | 23.925 | 2192.015 | 526681.563 | | 3897.44357 |
| 764 | 21.89 | 2191.903 | 167538 | | 1239.7812 |
| 765 | 50.864 | 2191.723 | 32037.535 | | 237.077759 |
| 766 | 47.244 | 2188.395 | 251956.906 | | 1864.4811 |
| 767 | 32.649 | 2188.058 | 78885.688 | | 583.754091 |
| 768 | 24.014 | 2186.089 | 116699.281 | | 863.574679 |
| 769 | 29.978 | 2184.703 | 233078.734 | | 1724.78263 |
| 770 | 39.09 | 2183.992 | 1648277.63 | | 12197.2544 |
| 771 | 50.193 | 2178.759 | 33248.797 | | 246.041098 |
| 772 | 54.895 | 2176.709 | 42103.77 | | 311.567898 |
| 773 | 21.423 | 2175.984 | 37351.434 | | 276.400612 |
| 774 | 51.031 | 2175.813 | 37432.242 | | 276.998591 |
| 775 | 28.214 | 2175.135 | 523507.813 | 4308.36938 | 3873.95782 |
| 776 | 55.139 | 2174.695 | 12196.703 | | 90.2556022 |
| 777 | 47.254 | 2172.427 | 139909.953 | | 1035.33365 |
| 778 | 24.955 | 2171.845 | 392253.938 | | 2902.67914 |
| 779 | 54.95 | 2170.835 | 83249.852 | | 616.048905 |
| 780 | 23.99 | 2170.06 | 2801978.75 | 16950.086 | 20734.6428 |
| 781 | 33.051 | 2169.816 | 115270.117 | | 852.998866 |
| 782 | 28.029 | 2169.083 | 41560.289 | | 307.546139 |
| 783 | 29.942 | 2168.741 | 50702.242 | | 375.196591 |
| 784 | 38.918 | 2168 | 625043.188 | | 4625.31959 |
| 785 | 47.105 | 2167.452 | 166361.109 | | 1231.07221 |
| 786 | 23.628 | 2166.176 | 62101.336 | | 459.549886 |
| 787 | 27.877 | 2163.994 | 17963.393 | | 132.929108 |
| 788 | 19.292 | 2163.03 | 26186.1 | | 193.77714 |
| 789 | 28.177 | 2159.127 | 165196.703 | 2003.94316 | 1222.4556 |
| 790 | 21.729 | 2157.014 | 371334.938 | | 2747.87854 |
| 791 | 50.303 | 2156.445 | 98210.313 | | 726.756316 |
| 792 | 24.927 | 2156.036 | 134981.219 | | 998.861021 |
| 793 | 54.905 | 2155.728 | 215793.328 | | 1596.87063 |
| 794 | 50.291 | 2153.919 | 356736.531 | | 2639.85033 |
| 795 | 33.038 | 2153.853 | 17994.725 | | 133.160965 |
| 796 | 24.073 | 2153.761 | 687819.375 | | 5089.86338 |
| 797 | 21.431 | 2151.975 | 13115.051 | | 97.0513774 |
| 798 | 47.014 | 2150.457 | 80450.156 | | 595.331154 |
| 799 | 24.985 | 2150.074 | 124303.188 | | 919.843591 |
| 800 | 23.697 | 2148.078 | 14427.769 | | 106.765491 |
| 801 | 47.017 | 2146.51 | 33126.008 | | 245.132459 |
| 802 | 39.031 | 2146.033 | 1517131 | | 11226.7694 |
| 803 | 28.033 | 2142.049 | 116757.086 | | 864.002436 |
| 804 | 50.317 | 2140.053 | 49425.555 | | 365.749107 |
| 805 | 54.907 | 2139.687 | 131492.109 | | 973.041607 |
| 806 | 19.34 | 2139.264 | 16454.809 | | 121.765587 |
| 807 | 21.438 | 2138.028 | 170402.016 | | 1260.97492 |
| 808 | 49.775 | 2137.904 | 122028.063 | | 903.007666 |
| 809 | 51.01 | 2137.833 | 107543.789 | | 795.824039 |
| 810 | 24.98 | 2134.064 | 184060.313 | | 1362.04632 |
| 811 | 23.947 | 2133.997 | 14915.034 | | 110.371252 |
| 812 | 54.93 | 2133.748 | 448603.344 | | 3319.66475 |
| 813 | 23.791 | 2132.926 | 98156.297 | | 726.356598 |
| 814 | 33.034 | 2131.834 | 93856.258 | | 694.536309 |
| 815 | 49.73 | 2131.826 | 40604.926 | | 300.476452 |
| 816 | 19.309 | 2126.895 | 11998.439 | | 88.7884486 |
| 817 | 19.23 | 2125.959 | 13855.418 | | 102.530093 |
| 818 | 28.013 | 2125.696 | 179600.594 | | 1329.0444 |
| 819 | 29.033 | 2123.978 | 9567.942 | | 70.8027708 |
| 820 | 28.424 | 2123.872 | 38521.063 | | 285.055866 |
| 821 | 19.288 | 2123.304 | 41390.043 | | 306.286318 |
| 822 | 54.923 | 2117.754 | 676757.125 | | 5008.00273 |
| 823 | 50.182 | 2115.807 | 562227.938 | | 4160.48674 |
| 824 | 32.559 | 2113.632 | 14000.153 | | 103.601132 |
| 825 | 27.977 | 2110.026 | 15948.838 | | 118.021401 |
| 826 | 19.242 | 2109.934 | 16406.662 | | 121.409299 |
| 827 | 23.748 | 2108.995 | 14636.829 | | 108.312535 |
| 828 | 27.756 | 2106.99 | 38564.801 | | 285.379527 |
| 829 | 20.492 | 2105.879 | 76712.156 | | 567.669954 |
| 830 | 54.924 | 2101.744 | 308647.25 | | 2283.98965 |
| 831 | 21.456 | 2101.398 | 246869.797 | | 1826.8365 |
| 832 | 49.813 | 2099.871 | 64825.379 | | 479.707805 |
| 833 | 50.969 | 2099.86 | 21705.629 | | 160.621655 |
| 834 | 22.512 | 2098.949 | 30868.66 | | 228.428084 |
| 835 | 54.919 | 2095.774 | 1133086.88 | | 8384.84288 |
| 836 | 56.91 | 2094.771 | 16988.391 | | 125.714093 |
| 837 | 50.601 | 2094.588 | 22926.068 | | 169.652903 |
| 838 | 23.784 | 2093.206 | 75977.586 | | 562.234136 |
| 839 | 28.084 | 2088.017 | 171200.609 | 1269.99462 | 1266.88451 |
| 840 | 19.264 | 2087.973 | 350660.688 | | 2594.88909 |
| 841 | 23.117 | 2086.65 | 12786.285 | | 94.618509 |
| 842 | 27.791 | 2085.045 | 199885.375 | | 1479.15178 |
| 843 | 20.269 | 2081.018 | 229086.047 | | 1695.23675 |
| 844 | 54.93 | 2079.801 | 993210.188 | | 7349.75539 |
| 845 | 47.595 | 2079.318 | 19015.662 | | 140.715899 |
| 846 | 56.839 | 2078.89 | 19401.766 | | 143.573068 |
| 847 | 49.785 | 2077.893 | 115355.531 | | 853.630929 |
| 848 | 21.811 | 2077.762 | 31063.748 | | 229.871735 |
| 849 | 52.075 | 2077.73 | 6613.918 | | 48.9429932 |
| 850 | 21.41 | 2076.984 | 175348.891 | | 1297.58179 |
| 851 | 51.543 | 2071.648 | 7766.515 | | 57.472211 |
| 852 | 23.759 | 2071.002 | 133591.328 | | 988.575827 |
| 853 | 21.424 | 2070.003 | 109701.07 | | 811.787918 |
| 854 | 51.254 | 2069.625 | 61727.754 | | 456.78538 |
| 855 | 27.817 | 2069.09 | 222562.547 | | 1646.96285 |
| 856 | 20.532 | 2067.921 | 477863.719 | | 3536.19152 |
| 857 | 27.607 | 2063.948 | 71420.422 | | 528.511123 |
| 858 | 54.94 | 2063.804 | 166864.938 | | 1234.80054 |
| 859 | 55.581 | 2062.783 | 10409.057 | | 77.0270218 |
| 860 | 22.44 | 2059.045 | 31675.367 | | 234.397716 |
| 861 | 54.9 | 2057.817 | 1045255.63 | | 7734.89163 |
| 862 | 20.802 | 2056.804 | 23316.27 | | 172.540398 |
| 863 | 47.411 | 2056.32 | 114767.031 | | 849.276029 |
| 864 | 23.834 | 2056.014 | 75674.141 | | 559.988643 |
| 865 | 52.099 | 2054.689 | 8212.415 | | 60.771871 |
| 866 | 51.141 | 2052.646 | 7170.431 | | 53.0611894 |
| 867 | 23.596 | 2050.953 | 30659.434 | | 226.879812 |
| 868 | 23.173 | 2049.013 | 179035.266 | | 1324.86097 |
| 869 | 51.154 | 2047.635 | 93990.273 | | 695.52802 |
| 870 | 27.801 | 2047.175 | 411581 | 1703.37702 | 3045.6994 |
| 871 | 28.43 | 2045.938 | 76368.227 | | 565.12488 |
| 872 | 28.078 | 2041.99 | 11063.896 | | 81.8728304 |
| 873 | 54.923 | 2041.771 | 361323.031 | | 2673.79043 |
| 874 | 21.426 | 2039.213 | 401444.531 | | 2970.68953 |
| 875 | 40.128 | 2036.857 | 62552.297 | | 462.886998 |
| 876 | 40.967 | 2036.734 | 16498.773 | | 122.09092 |
| 877 | 19.377 | 2035.142 | 119280.563 | | 882.676166 |
| 878 | 20.833 | 2034.878 | 107200.859 | | 793.286357 |
| 879 | 51.167 | 2031.663 | 179833.078 | | 1330.76478 |
| 880 | 27.821 | 2031.125 | 226310.281 | | 1674.69608 |
| 881 | 49.933 | 2030.735 | 8329.54 | | 61.638596 |
| 882 | 20.301 | 2029.874 | 59304.598 | | 438.854025 |
| 883 | 27.841 | 2027.979 | 24592.67 | | 181.985758 |
| 884 | 27.615 | 2025.995 | 173888.672 | | 1286.77617 |
| 885 | 21.281 | 2023.988 | 15667.275 | | 115.937835 |
| 886 | 54.897 | 2019.862 | 339981.563 | | 2515.86357 |
| 887 | 47.368 | 2018.356 | 144909.141 | | 1072.32764 |
| 888 | 20.769 | 2017.852 | 41804.027 | | 309.3498 |
| 889 | 50.41 | 2017.309 | 23882.344 | | 176.729346 |
| 890 | 52.145 | 2016.741 | 25066.926 | | 185.495252 |
| 891 | 51.168 | 2015.674 | 15682.338 | | 116.049301 |
| 892 | 21.462 | 2014.872 | 676609.125 | | 5006.90753 |
| 893 | 23.598 | 2013.999 | 118936.234 | | 880.128132 |
| 894 | 27.425 | 2013.957 | 10481.886 | | 77.5659564 |
| 895 | 27.626 | 2009.933 | 150281.469 | | 1112.08287 |
| 896 | 51.219 | 2009.681 | 269662.813 | | 1995.50482 |
| 897 | 51.765 | 2008.693 | 8050.752 | | 59.5755648 |
| 898 | 27.027 | 2008.114 | 36968.852 | | 273.569505 |
| 899 | 49.949 | 2007.869 | 263181.844 | | 1947.54565 |
| 900 | 53.968 | 2007.765 | 23462.338 | | 173.621301 |
| 901 | 47.566 | 2003.37 | 5718.014 | | 42.3133036 |
| 902 | 20.834 | 1996.814 | 335825.125 | | 2485.10593 |
| 903 | 49.681 | 1995.723 | 5319.325 | | 39.363005 |
| 904 | 50.085 | 1995.688 | 7241.29 | | 53.585546 |
| 905 | 21.049 | 1994.926 | 32653.094 | | 241.632896 |
| 906 | 27.528 | 1993.98 | 20820.145 | | 154.069073 |
| 907 | 51.008 | 1993.068 | 187387.828 | | 1386.66993 |
| 908 | 49.882 | 1991.781 | 242254.469 | | 1792.68307 |
| 909 | 27.745 | 1989.977 | 116543.305 | | 862.420457 |
| 910 | 39.9 | 1989.431 | 174175.406 | | 1288.898 |
| 911 | 23.409 | 1987.045 | 367932.188 | | 2722.69819 |
| 912 | 27.172 | 1985.949 | 13387.146 | | 99.0648804 |
| 913 | 20.148 | 1980.776 | 40316.543 | | 298.342418 |
| 914 | 47.304 | 1980.392 | 93012.695 | | 688.293943 |
| 915 | 35.172 | 1977.731 | 80382.313 | | 594.829116 |
| 916 | 27.666 | 1975.777 | 70365.492 | | 520.704641 |
| 917 | 47.124 | 1975.433 | 105832.047 | | 783.157148 |
| 918 | 39.641 | 1972.397 | 211920.281 | | 1568.21008 |
| 919 | 21.264 | 1971.932 | 24054.641 | | 178.004343 |
| 920 | 41.424 | 1971.717 | 16212.052 | | 119.969185 |
| 921 | 40.892 | 1971.699 | 25774.264 | | 190.729554 |
| 922 | 51.25 | 1971.598 | 203815.859 | | 1508.23736 |
| 923 | 23.437 | 1971.004 | 533845.5 | | 3950.4567 |
| 924 | 41.952 | 1970.701 | 11751.374 | | 86.9601676 |
| 925 | 49.899 | 1969.877 | 510840.781 | | 3780.22178 |
| 926 | 54.004 | 1969.775 | 110049.828 | | 814.368727 |
| 927 | 52.803 | 1969.714 | 3832.406 | | 28.3598044 |
| 928 | 40.409 | 1967.647 | 566741.125 | | 4193.88433 |
| 929 | 51.49 | 1966.766 | 8687.899 | | 64.2904526 |
| 930 | 20.143 | 1964.822 | 68999.875 | | 510.599075 |
| 931 | 27.226 | 1963.76 | 22017.5 | | 162.9295 |
| 932 | 35.592 | 1961.849 | 215947.656 | | 1598.01265 |
| 933 | 20.186 | 1958.815 | 182788.188 | | 1352.63259 |
| 934 | 25.235 | 1955.974 | 34034.582 | | 251.855907 |
| 935 | 34.906 | 1955.842 | 23583.658 | | 174.519069 |
| 936 | 35.204 | 1954.875 | 23046.27 | | 170.542398 |
| 937 | 20.225 | 1954.822 | 104457.664 | | 772.986714 |
| 938 | 27.654 | 1954.071 | 25012.77 | | 185.094498 |
| 939 | 49.914 | 1953.531 | 149496.656 | | 1106.27525 |
| 940 | 23.846 | 1951.907 | 11188.573 | | 82.7954402 |
| 941 | 40.048 | 1951.671 | 405308.781 | | 2999.28498 |
| 942 | 49.519 | 1951.462 | 32885.328 | | 243.351427 |
| 943 | 28.011 | 1950.926 | 80130.508 | | 592.965759 |
| 944 | 29.888 | 1950.847 | 95179.586 | | 704.328936 |
| 945 | 21.331 | 1949.954 | 15990.713 | | 118.331276 |
| 946 | 19.872 | 1949.895 | 8138.52 | | 60.225048 |
| 947 | 23.457 | 1949.102 | 3147247 | | 23289.6278 |
| 948 | 27.164 | 1947.721 | 65290.855 | | 483.152327 |
| 949 | 35.176 | 1945.87 | 48158.836 | | 356.375386 |
| 950 | 51.644 | 1945.533 | 5578.079 | | 41.2777846 |
| 951 | 28.503 | 1945.117 | 26344.092 | | 194.946281 |
| 952 | 20.19 | 1942.355 | 176231.625 | | 1304.11403 |
| 953 | 36.009 | 1939.849 | 754929.75 | | 5586.48015 |
| 954 | 21.083 | 1939.728 | 83881.531 | | 620.723329 |
| 955 | 27.622 | 1937.032 | 122677.93 | | 907.816682 |
| 956 | 20.174 | 1935.815 | 14819.995 | | 109.667963 |
| 957 | 55.67 | 1935.509 | 1866.308 | | 13.8106792 |
| 958 | 19.929 | 1934.88 | 185292.125 | | 1371.16173 |
| 959 | 27.6 | 1933.946 | 21970.883 | | 162.584534 |
| 960 | 21.312 | 1933.467 | 120517.273 | | 891.82782 |
| 961 | 23.44 | 1933.132 | 1264035.38 | | 9353.86178 |
| 962 | 49.882 | 1931.835 | 169525.344 | | 1254.48755 |
| 963 | 53.987 | 1931.539 | 7912.462 | | 58.5522188 |
| 964 | 32.897 | 1930.835 | 60372.492 | | 446.756441 |
| 965 | 40.453 | 1929.642 | 1351748.75 | | 10002.9408 |
| 966 | 49.985 | 1928.736 | 30871.664 | | 228.450314 |
| 967 | 19.413 | 1928.014 | 24680.447 | | 182.635308 |
| 968 | 22.284 | 1925.639 | 28804.176 | | 213.150902 |
| 969 | 36.02 | 1923.804 | 788593 | | 5835.5882 |
| 970 | 32.585 | 1923.744 | 18391.229 | | 136.095095 |
| 971 | 51.474 | 1923.542 | 22426.631 | | 165.957069 |
| 972 | 21.155 | 1921.892 | 25471.947 | | 188.492408 |
| 973 | 20.197 | 1920.513 | 484588.406 | | 3585.9542 |
| 974 | 35.145 | 1917.819 | 151947.547 | | 1124.41185 |
| 975 | 20.275 | 1916.865 | 592958 | | 4387.8892 |
| 976 | 32.944 | 1914.85 | 53011.285 | | 392.283509 |
| 977 | 39.532 | 1913.055 | 359803.531 | | 2662.54613 |
| 978 | 27.873 | 1912.956 | 171374.188 | | 1268.16899 |
| 979 | 49.886 | 1912.77 | 90604.273 | | 670.47162 |
| 980 | 23.536 | 1911.131 | 16659075 | | 123277.155 |
| 981 | 19.165 | 1909.169 | 16568.082 | | 122.603807 |
| 982 | 35.112 | 1907.913 | 132959.188 | | 983.897991 |
| 983 | 51.45 | 1907.405 | 55725.84 | | 412.371216 |
| 984 | 20.208 | 1904.837 | 417636.094 | | 3090.5071 |
| 985 | 19.974 | 1901.937 | 12937.851 | | 95.7400974 |
| 986 | 36.035 | 1901.931 | 1493067.88 | | 11048.7023 |
| 987 | 27.44 | 1901.067 | 35484.109 | | 262.582407 |
| 988 | 21.174 | 1898.892 | 49621.984 | | 367.202682 |
| 989 | 26.836 | 1897.634 | 51880.426 | | 383.915152 |
| 990 | 22.569 | 1895.801 | 31370.744 | | 232.143506 |
| 991 | 50.839 | 1895.655 | 14071.792 | | 104.131261 |
| 992 | 19.364 | 1893.904 | 10081.896 | | 74.6060304 |
| 993 | 20.096 | 1893.708 | 16126.631 | | 119.337069 |
| 994 | 23.44 | 1893.144 | 138012.906 | | 1021.2955 |
| 995 | 33.628 | 1892.875 | 666429.375 | | 4931.57738 |
| 996 | 40.178 | 1891.728 | 965003.063 | | 7141.02267 |
| 997 | 23.747 | 1890.939 | 199265.828 | | 1474.56713 |
| 998 | 28.18 | 1889.913 | 10303.565 | | 76.246381 |
| 999 | 51.81 | 1886.097 | 32181.391 | | 238.142293 |
| 1000 | 49.855 | 1886.017 | 9415.063 | | 69.6714662 |
| 1001 | 19.928 | 1885.96 | 15694.74 | | 116.141076 |
| 1002 | 35.93 | 1885.943 | 758995.75 | | 5616.56855 |
| 1003 | 51.337 | 1885.571 | 114489.086 | | 847.219236 |
| 1004 | 20.18 | 1882.925 | 4423241 | | 32731.9834 |
| 1005 | 29.37 | 1882.543 | 68798.625 | | 509.109825 |
| 1006 | 35.135 | 1879.95 | 58723.824 | | 434.556298 |
| 1007 | 22.494 | 1879.257 | 20705.602 | | 153.221455 |
| 1008 | 34.349 | 1878.875 | 33668.203 | | 249.144702 |
| 1009 | 22.591 | 1876.914 | 37062.238 | | 274.260561 |
| 1010 | 21.725 | 1876.902 | 297599.3131 | 1550.63508 | 2202.23492 |
| 1011 | 32.986 | 1876.876 | 128455.727 | | 950.57238 |
| 1012 | 55.924 | 1875.624 | 2100.854 | | 15.5463196 |
| 1013 | 26.625 | 1875.501 | 105028.836 | | 777.213386 |
| 1014 | 27.296 | 1874.767 | 67852.57 | | 502.109018 |
| 1015 | 26.891 | 1873.891 | 93489.258 | | 691.820509 |
| 1016 | 21.431 | 1872.932 | 23245.711 | | 172.018261 |
| 1017 | 19.83 | 1872.86 | 146425.875 | | 1083.55148 |
| 1018 | 51.282 | 1869.592 | 141043.234 | | 1043.71993 |
| 1019 | 20.63 | 1867.838 | 31031.188 | | 229.630791 |
| 1020 | 27.992 | 1864.675 | 33520.336 | | 248.050486 |
| 1021 | 47.391 | 1864.278 | 57918.516 | | 428.597018 |
| 1022 | 20.099 | 1864.101 | 137271.891 | | 1015.81199 |
| 1023 | 35.869 | 1863.967 | 524659.438 | | 3882.47984 |
| 1024 | 27.239 | 1863.873 | 52788.363 | | 390.633886 |
| 1025 | 20.031 | 1863.141 | 139697.438 | | 1033.76104 |
| 1026 | 19.87 | 1860.926 | 13068.771 | | 96.7089054 |
| 1027 | 21.157 | 1860.926 | 81917.836 | | 606.191986 |
| 1028 | 26.764 | 1860.34 | 197601.531 | | 1462.25133 |
| 1029 | 27.282 | 1859.5 | 48407.289 | | 358.213939 |
| 1030 | 23.006 | 1859.03 | 193516.188 | | 1432.01979 |
| 1031 | 51.461 | 1858.652 | 43128.02 | | 319.147348 |
| 1032 | 22.605 | 1857.839 | 203838.828 | | 1508.40733 |
| 1033 | 19.267 | 1855.928 | 13138.126 | | 97.2221324 |
| 1034 | 20.105 | 1855.764 | 131766.063 | | 975.068866 |
| 1035 | 33.577 | 1854.908 | 733231.875 | | 5425.91588 |
| 1036 | 26.818 | 1851.8 | 200711.625 | | 1485.26603 |
| 1037 | 60.637 | 1848.46 | 6038.243 | | 44.6829982 |
| 1038 | 19.998 | 1848.263 | 99688.43 | | 737.694382 |
| 1039 | 27.542 | 1847.933 | 53558.961 | | 396.336311 |
| 1040 | 51.445 | 1847.586 | 194541.844 | | 1439.60965 |
| 1041 | 50.289 | 1846.06 | 144120.094 | | 1066.4887 |
| 1042 | 20.658 | 1845.847 | 131101.281 | | 970.149479 |
| 1043 | 26.69 | 1844.668 | 50529.566 | | 373.918788 |
| 1044 | 51.19 | 1844.662 | 11750.058 | | 86.9504292 |
| 1045 | 36.173 | 1843.622 | 40933.469 | | 302.907671 |
| 1046 | 50.239 | 1842.658 | 50739.762 | | 375.474239 |
| 1047 | 51.616 | 1842.648 | 61330.453 | | 453.845352 |
| 1048 | 22.565 | 1841.844 | 52639.223 | | 389.53025 |
| 1049 | 20.14 | 1839.786 | 104989.461 | | 776.922011 |
| 1050 | 26.58 | 1837.73 | 168274.531 | | 1245.23153 |
| 1051 | 27.295 | 1836.888 | 154800.984 | | 1145.52728 |
| 1052 | 19.861 | 1835.81 | 239974.203 | | 1775.8091 |
| 1053 | 26.859 | 1835.179 | 177662.859 | 3450.55729 | 1314.70516 |
| 1054 | 22.947 | 1834.847 | 112151.383 | | 829.920234 |
| 1055 | 22.831 | 1832.844 | 128282.289 | | 949.288939 |
| 1056 | 20.82 | 1831.312 | 39760.145 | | 294.225073 |
| 1057 | 51.429 | 1831.23 | 74989.969 | | 554.925771 |
| 1058 | 20.578 | 1829.857 | 18786.881 | | 139.022919 |
| 1059 | 50.43 | 1829.681 | 33509.582 | | 247.970907 |
| 1060 | 33.814 | 1829.529 | 37306.023 | | 276.06457 |
| 1061 | 49.958 | 1828.949 | 54472.137 | | 403.093814 |
| 1062 | 28.024 | 1827.115 | 55237.465 | | 408.757241 |
| 1063 | 50.335 | 1826.663 | 44625.695 | | 330.230143 |
| 1064 | 51.638 | 1826.652 | 57917.66 | | 428.590684 |
| 1065 | 47.405 | 1826.309 | 86090.477 | | 637.06953 |
| 1066 | 27.216 | 1825.887 | 48555.508 | | 359.310759 |
| 1067 | 20.099 | 1825.86 | 338040.688 | | 2501.50109 |
| 1068 | 23.404 | 1824.068 | 54327.395 | | 402.022723 |
| 1069 | 55.496 | 1823.55 | 1463.899 | | 10.8328526 |
| 1070 | 26.708 | 1822.845 | 185135.641 | | 1370.00374 |
| 1071 | 27.294 | 1821.908 | 36764.754 | | 272.05918 |
| 1072 | 26.419 | 1821.874 | 13089.423 | | 96.8617302 |
| 1073 | 51.474 | 1820.716 | 52165.934 | | 386.027912 |
| 1074 | 22.606 | 1819.804 | 580688.75 | | 4297.09675 |
| 1075 | 20.154 | 1817.761 | 1081253.88 | | 8001.27868 |
| 1076 | 37.543 | 1817.747 | 40002.078 | | 296.015377 |
| 1077 | 27.079 | 1813.82 | 933491.563 | | 6907.83757 |
| 1078 | 22.791 | 1812.882 | 126053.18 | | 932.793532 |
| 1079 | 60.619 | 1810.514 | 9529.108 | | 70.5153992 |
| 1080 | 27.679 | 1809.961 | 61372.824 | | 454.158898 |
| 1081 | 51.36 | 1809.662 | 166998.938 | | 1235.79214 |
| 1082 | 25.419 | 1808.785 | 99647.727 | | 737.39318 |
| 1083 | 32.362 | 1807.95 | 28583.354 | | 211.51682 |
| 1084 | 20.635 | 1807.641 | 329490.406 | | 2438.229 |
| 1085 | 22.31 | 1806.883 | 44412.402 | | 328.651775 |
| 1086 | 40.188 | 1805.588 | 418072.781 | | 3093.73858 |
| 1087 | 50.551 | 1804.673 | 858333.75 | | 6351.66975 |
| 1088 | 54.902 | 1804.666 | 19508.908 | | 144.365919 |
| 1089 | 47.258 | 1804.327 | 91234.57 | | 675.135818 |
| 1090 | 26.77 | 1803.88 | 266848.438 | | 1974.67844 |
| 1091 | 32.344 | 1801.343 | 78405.297 | | 580.199198 |
| 1092 | 30.974 | 1800.629 | 22093.713 | | 163.493476 |
| 1093 | 19.034 | 1800.052 | 117200.094 | | 867.280696 |
| 1094 | 20.101 | 1799.824 | 27975.473 | | 207.0185 |
| 1095 | 27.283 | 1798.711 | 106091.477 | | 785.07693 |
| 1096 | 26.789 | 1796.84 | 115289.117 | | 853.139466 |
| 1097 | 22.952 | 1794.908 | 403840.938 | | 2988.42294 |
| 1098 | 27.655 | 1793.973 | 137558.906 | | 1017.9359 |
| 1099 | 20.703 | 1793.821 | 42362.82 | | 313.484868 |
| 1100 | 26.926 | 1792.934 | 245127.938 | | 1813.94674 |
| 1101 | 25.423 | 1792.81 | 76797.188 | | 568.299191 |
| 1102 | 19.261 | 1790.148 | 27753.096 | | 205.37291 |
| 1103 | 26.987 | 1789.851 | 25226.754 | | 186.67798 |
| 1104 | 38.699 | 1789.61 | 59189.73 | | 438.004002 |
| 1105 | 26.216 | 1788.907 | 15748.181 | | 116.536539 |
| 1106 | 50.525 | 1788.693 | 893111.313 | | 6609.02372 |
| 1107 | 47.617 | 1788.321 | 6225.253 | | 46.0668722 |
| 1108 | 26.718 | 1787.886 | 220469.656 | | 1631.47545 |
| 1109 | 32.412 | 1785.707 | 138173.328 | | 1022.48263 |
| 1110 | 47.08 | 1783.395 | 13617.989 | | 100.773119 |
| 1111 | 24.05 | 1782.919 | 130107.336 | | 962.794286 |
| 1112 | 26.719 | 1779.838 | 13206.108 | | 97.7251992 |
| 1113 | 26.461 | 1778.925 | 77194.57 | | 571.239818 |
| 1114 | 19.165 | 1778.824 | 6165.233 | | 45.6227242 |
| 1115 | 19.281 | 1777.115 | 29204.076 | | 216.110162 |
| 1116 | 26.899 | 1775.901 | 368163.375 | | 2724.40898 |
| 1117 | 22.755 | 1775.826 | 568114.875 | | 4204.05008 |
| 1118 | 50.249 | 1772.72 | 125865.164 | | 931.402214 |
| 1119 | 51.599 | 1772.686 | 30238.418 | | 223.764293 |
| 1120 | 19.906 | 1772.576 | 13414.021 | | 99.2637554 |
| 1121 | 26.14 | 1771.789 | 20342.643 | | 150.535558 |
| 1122 | 60.659 | 1771.538 | 4335.641 | | 32.0837434 |
| 1123 | 25.395 | 1770.678 | 364467.063 | | 2697.05627 |
| 1124 | 32.44 | 1769.84 | 87244.375 | | 645.608375 |
| 1125 | 26.406 | 1768.832 | 73680.18 | | 545.233332 |
| 1126 | 27.285 | 1768 | 51965.754 | | 384.54658 |
| 1127 | 40.282 | 1767.639 | 1154890.88 | | 8546.19248 |
| 1128 | 23.421 | 1767.108 | 56689.273 | | 419.50062 |
| 1129 | 22.915 | 1767.084 | 339444.344 | | 2511.88815 |
| 1130 | 50.388 | 1766.72 | 1244047.25 | | 9205.94965 |
| 1131 | 54.846 | 1766.713 | 18705.223 | | 138.41865 |
| 1132 | 46.966 | 1766.364 | 27324.01 | | 202.197674 |
| 1133 | 26.826 | 1766.124 | 676256.625 | | 5004.29903 |
| 1134 | 27.6 | 1765.883 | 24261.887 | | 179.537964 |
| 1135 | 19.883 | 1764.796 | 43908.137 | | 324.920214 |
| 1136 | 30.95 | 1763.615 | 10578.803 | | 78.2831422 |
| 1137 | 32.353 | 1763.538 | 197962.688 | | 1464.92389 |
| 1138 | 52.236 | 1762.263 | 24308.041 | | 179.879503 |
| 1139 | 19.154 | 1761.794 | 275627.063 | | 2039.64027 |
| 1140 | 32.005 | 1761.361 | 33788.863 | | 250.037586 |
| 1141 | 26.342 | 1760.756 | 28904.85 | | 213.89589 |
| 1142 | 22.781 | 1759.833 | 111226.188 | | 823.073791 |
| 1143 | 26.285 | 1756.792 | 65174.434 | | 482.290812 |
| 1144 | 26.977 | 1755.048 | 485130.938 | | 3589.96894 |
| 1145 | 25.433 | 1754.849 | 154997.719 | | 1146.98312 |
| 1146 | 19.568 | 1752.837 | 9993.905 | | 73.954897 |
| 1147 | 26.365 | 1752.747 | 8704.931 | | 64.4164894 |
| 1148 | 34.142 | 1752.717 | 5691.779 | | 42.1191646 |
| 1149 | 38.509 | 1751.662 | 40417.168 | | 299.087043 |
| 1150 | 51.038 | 1751.619 | 21932.201 | | 162.298287 |
| 1151 | 51.655 | 1750.712 | 48846.887 | | 361.466964 |
| 1152 | 50.127 | 1750.711 | 321034.5 | | 2375.6553 |
| 1153 | 22.842 | 1750.641 | 298645.875 | | 2209.97948 |
| 1154 | 26.646 | 1749.91 | 151839.859 | | 1123.61496 |
| 1155 | 19.696 | 1749.416 | 18542.609 | | 137.215307 |
| 1156 | 47.314 | 1749.411 | 3008.298 | | 22.2614052 |
| 1157 | 27.975 | 1748.147 | 81581.477 | | 603.70293 |
| 1158 | 32.363 | 1747.731 | 89702.695 | | 663.799943 |
| 1159 | 55.946 | 1747.592 | 6465.131 | | 47.8419694 |
| 1160 | 26.449 | 1746.84 | 61283.23 | | 453.495902 |
| 1161 | 35.783 | 1745.692 | 9288.554 | | 68.7352996 |
| 1162 | 56.832 | 1745.568 | 6267.448 | | 46.3791152 |
| 1163 | 36.089 | 1745.067 | 41401.465 | | 306.370841 |
| 1164 | 22.138 | 1744.813 | 35727.973 | | 264.387 |
| 1165 | 36.42 | 1744.373 | 27876.617 | | 206.286966 |
| 1166 | 26.218 | 1740.802 | 26704.641 | | 197.614343 |
| 1167 | 20.752 | 1740.022 | 166723.391 | | 1233.75309 |
| 1168 | 23.583 | 1738.825 | 94085.523 | | 696.23287 |
| 1169 | 26.387 | 1738.795 | 81945.344 | | 606.395546 |
| 1170 | 26.797 | 1737.895 | 254024.781 | 2608.25236 | 1879.78338 |
| 1171 | 22.79 | 1737.77 | 751127.625 | | 5558.34443 |
| 1172 | 26.66 | 1736.361 | 112063.227 | | 829.26788 |
| 1173 | 19.845 | 1734.781 | 21712.186 | | 160.670176 |
| 1174 | 21.937 | 1734.705 | 26412.207 | | 195.450332 |
| 1175 | 26.121 | 1733.55 | 44750.543 | | 331.154018 |
| 1176 | 25.448 | 1732.885 | 675405.813 | | 4998.00302 |
| 1177 | 26.799 | 1731.178 | 590140.125 | | 4367.03693 |
| 1178 | 51.061 | 1730.476 | 53972.723 | | 399.39815 |
| 1179 | 40.062 | 1729.677 | 777807.625 | | 5755.77643 |
| 1180 | 50.108 | 1728.761 | 243423.203 | | 1801.3317 |
| 1181 | 51.671 | 1728.713 | 12968.45 | | 95.96653 |
| 1182 | 33.569 | 1727.797 | 336655.563 | | 2491.25117 |
| 1183 | 32.353 | 1725.586 | 166671.5 | | 1233.3691 |
| 1184 | 18.942 | 1724.448 | 9412.43 | | 69.651982 |
| 1185 | 52.194 | 1724.241 | 44839.199 | | 331.810073 |
| 1186 | 31.985 | 1723.47 | 62237.195 | | 460.555243 |
| 1187 | 26.609 | 1722.557 | 453472.031 | | 3355.69303 |
| 1188 | 19.582 | 1720.78 | 74309.211 | | 549.888161 |
| 1189 | 27.777 | 1719.806 | 8670.013 | | 64.1580962 |
| 1190 | 26.311 | 1718.82 | 258650.984 | | 1914.01728 |
| 1191 | 22.038 | 1718.203 | 26217.236 | | 194.007546 |
| 1192 | 22.806 | 1717.892 | 71530.109 | | 529.322807 |
| 1193 | 22.835 | 1717.027 | 79037.438 | | 584.877041 |
| 1194 | 25.359 | 1716.873 | 78706.383 | | 582.427234 |
| 1195 | 20.092 | 1716.75 | 98706.563 | | 730.428566 |
| 1196 | 22.344 | 1716.589 | 114901.781 | | 850.273179 |
| 1197 | 56.213 | 1715.625 | 8737.123 | | 64.6547102 |
| 1198 | 51.077 | 1714.783 | 12487.07 | | 92.404318 |
| 1199 | 26.44 | 1712.803 | 97550.773 | | 721.87572 |
| 1200 | 21.961 | 1712.729 | 167742.406 | | 1241.2938 |
| 1201 | 33.485 | 1711.815 | 325279.094 | | 2407.0653 |
| 1202 | 19.769 | 1709.765 | 8169.03 | | 60.450822 |
| 1203 | 26.903 | 1708.88 | 616257 | | 4560.3018 |
| 1204 | 37.1 | 1707.723 | 573278.438 | | 4242.26044 |
| 1205 | 22.128 | 1706.846 | 147145.969 | | 1088.88017 |
| 1206 | 51.017 | 1706.819 | 8774.919 | | 64.9344006 |
| 1207 | 33.49 | 1705.824 | 107182.141 | | 793.147843 |
| 1208 | 26.213 | 1702.549 | 107273.141 | | 793.821243 |
| 1209 | 19.719 | 1701.773 | 10910.079 | | 80.7345846 |
| 1210 | 19.004 | 1701.117 | 286517 | | 2120.2258 |
| 1211 | 26.439 | 1700.815 | 121645.578 | | 900.177277 |
| 1212 | 22.605 | 1700.526 | 452406.844 | | 3347.81065 |
| 1213 | 31.892 | 1698.717 | 236323.828 | | 1748.79633 |
| 1214 | 26.019 | 1697.79 | 32841.801 | | 243.029327 |
| 1215 | 26.766 | 1697.722 | 293863.219 | | 2174.58782 |
| 1216 | 26.392 | 1696.803 | 58345.828 | | 431.759127 |
| 1217 | 22.005 | 1696.517 | 226208.672 | | 1673.94417 |
| 1218 | 33.389 | 1695.815 | 116949.117 | | 865.423466 |
| 1219 | 22.263 | 1693.709 | 34169.855 | | 252.856927 |
| 1220 | 20.506 | 1693.689 | 39384.203 | | 291.443102 |
| 1221 | 22.614 | 1693.346 | 26959.088 | | 199.497251 |
| 1222 | 26.76 | 1692.798 | 553402.438 | | 4095.17804 |
| 1223 | 51.073 | 1692.162 | 64894.574 | | 480.219848 |
| 1224 | 36.441 | 1691.753 | 474631.719 | | 3512.27472 |
| 1225 | 38.476 | 1691.74 | 71431.195 | | 528.590843 |
| 1226 | 33.701 | 1689.84 | 967976.563 | | 7163.02657 |
| 1227 | 20.188 | 1688.745 | 58794.035 | | 435.075859 |
| 1228 | 31.275 | 1688.712 | 23481.848 | | 173.765675 |
| 1229 | 22.111 | 1687.759 | 52805.07 | | 390.757518 |
| 1230 | 19.519 | 1687.737 | 8106.24 | | 59.986176 |
| 1231 | 32.032 | 1687.441 | 57712.715 | | 427.074091 |
| 1232 | 27.427 | 1686.789 | 19094.982 | | 141.302867 |
| 1233 | 26.916 | 1686.781 | 15861.432 | | 117.374597 |
| 1234 | 30.394 | 1686.748 | 8055.863 | | 59.6133862 |
| 1235 | 52.123 | 1686.612 | 18216.238 | | 134.800161 |
| 1236 | 20.925 | 1685.791 | 14135.824 | | 104.605098 |
| 1237 | 26.848 | 1684.778 | 1528617.38 | | 11311.7686 |
| 1238 | 51.06 | 1683.539 | 7311.187 | | 54.1027838 |
| 1239 | 56.025 | 1681.469 | 1456.791 | | 10.7802534 |
| 1240 | 26.327 | 1680.917 | 562451.125 | | 4162.13833 |
| 1241 | 50.711 | 1680.552 | 14415.57 | | 106.675218 |
| 1242 | 23.374 | 1680.067 | 13454.194 | | 99.5610356 |
| 1243 | 22.867 | 1680.06 | 1558934.5 | | 11536.1153 |
| 1244 | 22.348 | 1678.716 | 915839.25 | | 6777.21045 |
| 1245 | 20.024 | 1676.707 | 15076.088 | | 111.563051 |
| 1246 | 51.162 | 1675.553 | 15218.188 | | 112.614591 |
| 1247 | 21.999 | 1674.767 | 657963.75 | | 4868.93175 |
| 1248 | 26.48 | 1674.765 | 332114.281 | | 2457.64568 |
| 1249 | 35.911 | 1674.743 | 13065.354 | | 96.6836196 |
| 1250 | 33.635 | 1673.848 | 621973.75 | | 4602.60575 |
| 1251 | 37.217 | 1673.808 | 10630.163 | | 78.6632062 |
| 1252 | 31.406 | 1673.712 | 59847.359 | | 442.870457 |
| 1253 | 60.508 | 1673.41 | 3005.677 | | 22.2420098 |
| 1254 | 26.223 | 1673.293 | 89194.797 | | 660.041498 |
| 1255 | 20.472 | 1672.139 | 5100.346 | | 37.7425604 |
| 1256 | 37.138 | 1669.765 | 1178668.63 | | 8722.14783 |
| 1257 | 21.202 | 1669.559 | 36920.801 | | 273.213927 |
| 1258 | 51.231 | 1669.502 | 17024.83 | | 125.983742 |
| 1259 | 51.002 | 1668.263 | 32127.834 | | 237.745972 |
| 1260 | 22.682 | 1667.961 | 109042.328 | | 806.913227 |
| 1261 | 19.391 | 1667.748 | 14688.896 | | 108.69783 |
| 1262 | 26.233 | 1664.869 | 140821.266 | | 1042.07737 |
| 1263 | 50.726 | 1664.583 | 47740.727 | | 353.28138 |
| 1264 | 19.792 | 1663.743 | 15229.608 | | 112.699099 |
| 1265 | 23.647 | 1662.778 | 25791.463 | | 190.856826 |
| 1266 | 22.348 | 1662.716 | 194397.094 | | 1438.5385 |
| 1267 | 32.006 | 1662.609 | 175913.266 | | 1301.75817 |
| 1268 | 22.899 | 1661.776 | 484126.406 | | 3582.5354 |
| 1269 | 26.062 | 1659.819 | 69192.805 | | 512.026757 |
| 1270 | 26.413 | 1658.822 | 156914.141 | | 1161.16464 |
| 1271 | 22.01 | 1658.774 | 394689.219 | | 2920.70022 |
| 1272 | 25.845 | 1658.703 | 63723.551 | | 471.554277 |
| 1273 | 33.461 | 1657.828 | 80278.719 | | 594.062521 |
| 1274 | 20.167 | 1657.767 | 21783.213 | | 161.195776 |
| 1275 | 26.24 | 1655.771 | 115510.375 | | 854.776775 |
| 1276 | 25.813 | 1655.737 | 9132.748 | | 67.5823352 |
| 1277 | 20.067 | 1655.57 | 79395.461 | | 587.526411 |
| 1278 | 33.143 | 1654.782 | 45294.531 | | 335.179529 |
| 1279 | 26.579 | 1654.285 | 154289.203 | | 1141.7401 |
| 1280 | 39.156 | 1653.766 | 4578.064 | | 33.8776736 |
| 1281 | 51.132 | 1653.527 | 58067.93 | | 429.702682 |
| 1282 | 36.793 | 1653.059 | 534220.188 | | 3953.22939 |
| 1283 | 19.419 | 1652.746 | 15048.502 | | 111.358915 |
| 1284 | 33.485 | 1651.875 | 413598 | | 3060.6252 |
| 1285 | 34.676 | 1651.855 | 16018.068 | | 118.533703 |
| 1286 | 54.087 | 1651.453 | 14638.714 | | 108.326484 |
| 1287 | 19.538 | 1649.803 | 85486.398 | | 632.599345 |
| 1288 | 26.166 | 1649.782 | 13777.515 | | 101.953611 |
| 1289 | 22.145 | 1649.26 | 149952.375 | | 1109.64758 |
| 1290 | 45.247 | 1649.172 | 16909.164 | | 125.127814 |
| 1291 | 26.493 | 1647.797 | 148031.422 | | 1095.43252 |
| 1292 | 27.777 | 1647.787 | 11980.709 | | 88.6572466 |
| 1293 | 19.142 | 1647.559 | 26187.014 | | 193.783904 |
| 1294 | 60.612 | 1647.414 | 8742.313 | | 64.6931162 |
| 1295 | 20.618 | 1645.815 | 32788.332 | | 242.633657 |
| 1296 | 22.892 | 1645.794 | 215293.266 | | 1593.17017 |
| 1297 | 39.588 | 1644.614 | 24574.563 | | 181.851766 |
| 1298 | 40.996 | 1644.614 | 11609.523 | | 85.9104702 |
| 1299 | 41.529 | 1644.612 | 19104.215 | | 141.371191 |
| 1300 | 26.136 | 1643.782 | 40699.555 | | 301.176707 |
| 1301 | 50.713 | 1642.598 | 458311.781 | | 3391.50718 |
| 1302 | 22.368 | 1640.664 | 1787474 | | 13227.3076 |
| 1303 | 26.149 | 1639.423 | 79805.805 | | 590.562957 |
| 1304 | 20.164 | 1638.929 | 446489.313 | | 3304.02092 |
| 1305 | 31.575 | 1637.789 | 17758.281 | | 131.411279 |
| 1306 | 19.439 | 1637.762 | 13106.555 | | 96.988507 |
| 1307 | 33.079 | 1637.729 | 101983 | | 754.6742 |
| 1308 | 35.229 | 1637.691 | 8278.406 | | 61.2602044 |
| 1309 | 56.178 | 1637.442 | 1820.801 | | 13.4739274 |
| 1310 | 22.209 | 1636.865 | 1771329.38 | | 13107.8374 |
| 1311 | 19.979 | 1636.77 | 55767.305 | | 412.678057 |
| 1312 | 26.466 | 1636.56 | 493809.594 | | 3654.191 |
| 1313 | 33.565 | 1635.841 | 15748.322 | | 116.537583 |
| 1314 | 20.32 | 1635.767 | 14488.574 | | 107.215448 |
| 1315 | 26.222 | 1634.903 | 57033.582 | | 422.048507 |
| 1316 | 26.766 | 1634.835 | 31530.076 | | 233.322562 |
| 1317 | 30.976 | 1634.755 | 38005.172 | | 281.238273 |
| 1318 | 26.945 | 1633.589 | 57031.723 | | 422.03475 |
| 1319 | 56.093 | 1633.539 | 4547.565 | | 33.651981 |
| 1320 | 19.445 | 1633.168 | 33451.688 | | 247.542491 |
| 1321 | 33.285 | 1632.811 | 344321.844 | | 2547.98165 |
| 1322 | 37.063 | 1631.805 | 1000832.06 | | 7406.15727 |
| 1323 | 51.14 | 1631.544 | 14658.414 | | 108.472264 |
| 1324 | 19.444 | 1630.801 | 9351.153 | | 69.1985322 |
| 1325 | 20.708 | 1629.062 | 41170.219 | | 304.659621 |
| 1326 | 31.555 | 1628.503 | 27959.01 | | 206.896674 |
| 1327 | 26.276 | 1627.183 | 152365.281 | | 1127.50308 |
| 1328 | 49.992 | 1626.622 | 129430.719 | | 957.787321 |
| 1329 | 51.37 | 1626.608 | 221118.734 | | 1636.27863 |
| 1330 | 23.667 | 1624.787 | 74086.891 | | 548.242993 |
| 1331 | 31.977 | 1624.65 | 151979.656 | | 1124.64945 |
| 1332 | 22.91 | 1623.802 | 813636.25 | | 6020.90825 |
| 1333 | 20.049 | 1623.721 | 48035.508 | | 355.462759 |
| 1334 | 26.115 | 1622.752 | 54070.082 | | 400.118607 |
| 1335 | 26.587 | 1622.569 | 78495.656 | | 580.867854 |
| 1336 | 31.614 | 1622.541 | 48132.473 | | 356.1803 |
| 1337 | 51.449 | 1622.516 | 42449.113 | | 314.123436 |
| 1338 | 19.446 | 1621.752 | 14741.807 | | 109.089372 |
| 1339 | 33.103 | 1621.727 | 169860.656 | | 1256.96885 |
| 1340 | 36.26 | 1621.714 | 10330.106 | | 76.4427844 |
| 1341 | 25.866 | 1620.713 | 85293.617 | | 631.172766 |
| 1342 | 48.018 | 1619.242 | 4286.134 | | 31.7173916 |
| 1343 | 54.868 | 1618.342 | 4299.275 | | 31.814635 |
| 1344 | 26.239 | 1617.782 | 276657.656 | | 2047.26665 |
| 1345 | 55.144 | 1617.691 | 7426.702 | | 54.9575948 |
| 1346 | 33.243 | 1616.145 | 397344.281 | | 2940.34768 |
| 1347 | 60.515 | 1615.419 | 12032.381 | | 89.0396194 |
| 1348 | 22.542 | 1614.932 | 209708.531 | | 1551.84313 |
| 1349 | 19.439 | 1614.786 | 43254.602 | | 320.084055 |
| 1350 | 36.176 | 1614.752 | 366360.5 | | 2711.0677 |
| 1351 | 22.89 | 1613.878 | 29076.914 | | 215.169164 |
| 1352 | 31.579 | 1612.537 | 33888.555 | | 250.775307 |
| 1353 | 50.626 | 1610.636 | 57666.223 | | 426.73005 |
| 1354 | 51.796 | 1610.61 | 5026.365 | | 37.195101 |
| 1355 | 49.488 | 1610.521 | 27563.52 | | 203.970048 |
| 1356 | 26.476 | 1609.682 | 249802.609 | | 1848.53931 |
| 1357 | 19.82 | 1607.438 | 22530.867 | | 166.728416 |
| 1358 | 40.415 | 1606.62 | 6549.202 | | 48.4640948 |
| 1359 | 51.431 | 1606.541 | 53775.559 | | 397.939137 |
| 1360 | 60.308 | 1606.455 | 20217.408 | | 149.608819 |
| 1361 | 45.153 | 1606.051 | 104704.188 | | 774.810991 |
| 1362 | 33.093 | 1605.74 | 107556.352 | | 795.917005 |
| 1363 | 52.135 | 1604.625 | 27542.211 | | 203.812361 |
| 1364 | 51.752 | 1604.623 | 19708.334 | | 145.841672 |
| 1365 | 50.377 | 1604.425 | 498449.438 | | 3688.52584 |
| 1366 | 25.879 | 1602.593 | 80256.023 | | 593.89457 |
| 1367 | 20.142 | 1602.029 | 283347.281 | | 2096.76988 |
| 1368 | 26.208 | 1601.483 | 174360.688 | | 1290.26909 |
| 1369 | 21.843 | 1600.709 | 71368.32 | | 528.125568 |
| 1370 | 33.841 | 1599.775 | 1946478.5 | | 14403.9409 |
| 1371 | 26.365 | 1595.636 | 99707.125 | | 737.832725 |
| 1372 | 55.052 | 1595.379 | 13544.353 | | 100.228212 |
| 1373 | 33.255 | 1594.838 | 133055.125 | | 984.607925 |
| 1374 | 19.115 | 1594.616 | 9341.319 | | 69.1257606 |
| 1375 | 19.447 | 1592.784 | 123089.523 | | 910.86247 |
| 1376 | 52.334 | 1590.926 | 19367.498 | | 143.319485 |
| 1377 | 32.656 | 1590.783 | 42339.105 | | 313.309377 |
| 1378 | 27.674 | 1590.767 | 29748.822 | | 220.141283 |
| 1379 | 31.605 | 1590.551 | 240303.016 | | 1778.24232 |
| 1380 | 51.487 | 1590.549 | 28733.951 | | 212.631237 |
| 1381 | 60.485 | 1590.45 | 6380.994 | | 47.2193556 |
| 1382 | 20.136 | 1589.801 | 129593.336 | | 958.990686 |
| 1383 | 26.161 | 1589.765 | 28449.627 | | 210.52724 |
| 1384 | 26.465 | 1588.799 | 144476.25 | | 1069.12425 |
| 1385 | 50.453 | 1588.666 | 91979.852 | | 680.650905 |
| 1386 | 19.006 | 1588.024 | 23308.154 | | 172.48034 |
| 1387 | 32.774 | 1587.746 | 61703.977 | | 456.60943 |
| 1388 | 25.703 | 1587.197 | 49110.512 | | 363.417789 |
| 1389 | 19.518 | 1585.876 | 20618.105 | | 152.573977 |
| 1390 | 51.336 | 1584.592 | 147183.422 | | 1089.15732 |
| 1391 | 31.605 | 1584.465 | 112075.383 | | 829.357834 |
| 1392 | 26.004 | 1584.374 | 152446.016 | | 1128.10052 |
| 1393 | 33.983 | 1583.763 | 1606523.13 | | 11888.2711 |
| 1394 | 19.229 | 1583.691 | 41368.191 | | 306.124613 |
| 1395 | 60.592 | 1583.443 | 10818.154 | | 80.0543396 |
| 1396 | 22.462 | 1583.401 | 51175.965 | | 378.702141 |
| 1397 | 23.455 | 1580.979 | 750475.688 | | 5553.52009 |
| 1398 | 22.879 | 1580.944 | 82073.273 | | 607.34222 |
| 1399 | 26.267 | 1579.821 | 460781.469 | 2618.98951 | 3409.78287 |
| 1400 | 20.062 | 1579.781 | 1163685.25 | | 8611.27085 |
| 1401 | 55.029 | 1579.406 | 35646.781 | | 263.786179 |
| 1402 | 19.223 | 1578.194 | 34980.523 | | 258.85587 |
| 1403 | 34.082 | 1577.542 | 1220426.38 | | 9031.15518 |
| 1404 | 19.43 | 1577.048 | 211572.891 | | 1565.63939 |
| 1405 | 52.329 | 1575.122 | 15242.363 | | 112.793486 |
| 1406 | 31.579 | 1574.581 | 56584.949 | | 418.728623 |
| 1407 | 26.771 | 1573.768 | 11005.183 | | 81.4383542 |
| 1408 | 26.34 | 1573.766 | 88419.383 | | 654.303434 |
| 1409 | 25.642 | 1571.653 | 18782.912 | | 138.993549 |
| 1410 | 32.726 | 1570.316 | 86676.258 | | 641.404309 |
| 1411 | 26.215 | 1569.753 | 18192.246 | | 134.62262 |
| 1412 | 52.301 | 1569.494 | 49528.332 | | 366.509657 |
| 1413 | 25.953 | 1568.74 | 99835.68 | | 738.784032 |
| 1414 | 51.381 | 1568.572 | 103803.664 | | 768.147114 |
| 1415 | 60.515 | 1568.367 | 17354.266 | | 128.421568 |
| 1416 | 33.2 | 1567.797 | 434310.063 | | 3213.89447 |
| 1417 | 20.165 | 1567.795 | 372919.156 | | 2759.60175 |
| 1418 | 35.532 | 1567.755 | 7017.221 | | 51.9274354 |
| 1419 | 34.952 | 1567.754 | 19015.121 | | 140.711895 |
| 1420 | 22.644 | 1567.696 | 14336.802 | | 106.092335 |
| 1421 | 19.412 | 1567.693 | 15762.474 | | 116.642308 |
| 1422 | 25.669 | 1565.649 | 107737.258 | | 797.255709 |
| 1423 | 31.653 | 1563.506 | 256880.891 | | 1900.91859 |
| 1424 | 55.037 | 1563.437 | 31753.439 | | 234.975449 |
| 1425 | 21.99 | 1562.683 | 442783.563 | | 3276.59837 |
| 1426 | 31.395 | 1562.551 | 49908.277 | | 369.32125 |
| 1427 | 45.126 | 1562.09 | 120262.977 | | 889.94603 |
| 1428 | 33.892 | 1561.784 | 4378570.5 | | 32401.4217 |
| 1429 | 26.083 | 1560.761 | 172583.031 | | 1277.11443 |
| 1430 | 54.978 | 1557.43 | 57674.145 | | 426.788673 |
| 1431 | 47.314 | 1557.371 | 2242.944 | | 16.5977856 |
| 1432 | 25.21 | 1556.715 | 21400.275 | | 158.362035 |
| 1433 | 19.157 | 1556.658 | 40701.605 | | 301.191877 |
| 1434 | 25.762 | 1555.668 | 10494.886 | | 77.6621564 |
| 1435 | 52.317 | 1553.479 | 107733.453 | | 797.227552 |
| 1436 | 31.602 | 1552.596 | 164191 | | 1215.0134 |
| 1437 | 60.548 | 1552.244 | 18100.105 | | 133.940777 |
| 1438 | 32.949 | 1552.029 | 108975.336 | | 806.417486 |
| 1439 | 51.293 | 1551.536 | 9862.231 | | 72.9805094 |
| 1440 | 20.335 | 1551.002 | 41674.418 | | 308.390693 |
| 1441 | 26.24 | 1550.756 | 123754.648 | | 915.784395 |
| 1442 | 25.661 | 1549.661 | 181339.203 | | 1341.9101 |
| 1443 | 31.599 | 1547.554 | 126639.313 | | 937.130916 |
| 1444 | 52.2 | 1547.002 | 3902.257 | | 28.8767018 |
| 1445 | 26.148 | 1546.781 | 397945.156 | | 2944.79415 |
| 1446 | 51.365 | 1546.585 | 141115.656 | | 1044.25585 |
| 1447 | 18.965 | 1546.009 | 8138.867 | | 60.2276158 |
| 1448 | 33.874 | 1545.796 | 2482156.75 | | 18367.96 |
| 1449 | 22.718 | 1545.666 | 96470.289 | | 713.880139 |
| 1450 | 25.71 | 1543.679 | 57332.781 | | 424.262579 |
| 1451 | 55.025 | 1541.204 | 81368.375 | | 602.125975 |
| 1452 | 33.187 | 1539.813 | 342826.781 | | 2536.91818 |
| 1453 | 22.859 | 1539.745 | 17030.666 | | 126.026928 |
| 1454 | 26.143 | 1538.782 | 47694.641 | | 352.940343 |
| 1455 | 52.335 | 1537.494 | 74524.922 | | 551.484423 |
| 1456 | 54.213 | 1537.361 | 3714.262 | | 27.4855388 |
| 1457 | 20.394 | 1536.787 | 47255.895 | | 349.693623 |
| 1458 | 60.536 | 1536.53 | 5179.255 | | 38.326487 |
| 1459 | 26.437 | 1535.394 | 206936.766 | | 1531.33207 |
| 1460 | 26.032 | 1534.45 | 21739.525 | | 160.872485 |
| 1461 | 25.657 | 1533.617 | 116876.422 | | 864.885523 |
| 1462 | 31.429 | 1533.537 | 35516.992 | | 262.825741 |
| 1463 | 32.719 | 1533.021 | 52503.387 | | 388.525064 |
| 1464 | 52.327 | 1531.486 | 189285.625 | | 1400.71363 |
| 1465 | 58.795 | 1531.259 | 3579.956 | | 26.4916744 |
| 1466 | 25.934 | 1530.757 | 53750.309 | | 397.752287 |
| 1467 | 33.111 | 1529.795 | 354812.281 | | 2625.61088 |
| 1468 | 51.31 | 1529.754 | 68137.281 | | 504.215879 |
| 1469 | 22.669 | 1529.696 | 97531.555 | | 721.733507 |
| 1470 | 25.708 | 1527.677 | 557475 | | 4125.315 |
| 1471 | 21.377 | 1525.647 | 95958.109 | | 710.090007 |
| 1472 | 31.604 | 1525.585 | 224192.984 | | 1659.02808 |
| 1473 | 21.109 | 1524.714 | 50138.973 | | 371.0284 |
| 1474 | 20.247 | 1524.422 | 26032.813 | | 192.642816 |
| 1475 | 21.84 | 1523.845 | 1315288.25 | | 9733.13305 |
| 1476 | 33.882 | 1523.824 | 2137835.75 | | 15819.9846 |
| 1477 | 22.403 | 1522.807 | 56880.883 | | 420.918534 |
| 1478 | 26.05 | 1522.795 | 225871.531 | | 1671.44933 |
| 1479 | 20.691 | 1522.736 | 65239.566 | | 482.772788 |
| 1480 | 32.659 | 1522.66 | 48612.965 | | 359.735941 |
| 1481 | 52.275 | 1521.537 | 11822.047 | | 87.4831478 |
| 1482 | 55.043 | 1519.4 | 112879.547 | | 835.308648 |
| 1483 | 19.173 | 1518.7 | 310531.188 | | 2297.93079 |
| 1484 | 45.108 | 1518.042 | 159780.984 | | 1182.37928 |
| 1485 | 42.505 | 1517.544 | 9947.583 | | 73.6121142 |
| 1486 | 32.712 | 1517.013 | 81436.086 | | 602.627036 |
| 1487 | 52.328 | 1515.49 | 260667 | | 1928.9358 |
| 1488 | 51.064 | 1515.057 | 40951.52 | | 303.041248 |
| 1489 | 25.304 | 1514.678 | 105488.594 | | 780.615596 |
| 1490 | 31.295 | 1514.467 | 213922.516 | | 1583.02662 |
| 1491 | 58.997 | 1514.391 | 2223.704 | | 16.4554096 |
| 1492 | 33.008 | 1513.778 | 31166.486 | | 230.631996 |
| 1493 | 51.393 | 1513.492 | 6028.764 | | 44.6128536 |
| 1494 | 25.684 | 1511.67 | 533613.25 | | 3948.73805 |
| 1495 | 59.581 | 1511.401 | 7321.546 | | 54.1794404 |
| 1496 | 20.163 | 1510.747 | 120274.336 | | 890.030086 |
| 1497 | 23.298 | 1510.736 | 31251.807 | | 231.263372 |
| 1498 | 52.276 | 1509.5 | 7264.465 | | 53.757041 |
| 1499 | 21.434 | 1509.107 | 22580.268 | | 167.093983 |
| 1500 | 26.035 | 1508.878 | 416968.781 | 2402.66451 | 3085.56898 |
| 1501 | 21.099 | 1508.716 | 26668.498 | | 197.346885 |
| 1502 | 22.714 | 1507.972 | 296770.906 | | 2196.1047 |
| 1503 | 33.166 | 1507.821 | 377186.125 | | 2791.17733 |
| 1504 | 19.343 | 1507.692 | 21455.111 | | 158.767821 |
| 1505 | 51.294 | 1507.543 | 59747.996 | | 442.13517 |
| 1506 | 25.715 | 1505.656 | 93126.773 | | 689.13812 |
| 1507 | 21.393 | 1504.976 | 224635.625 | | 1662.30363 |
| 1508 | 31.026 | 1504.421 | 61428.184 | | 454.568562 |
| 1509 | 55.107 | 1503.301 | 55199.215 | | 408.474191 |
| 1510 | 22.308 | 1500.84 | 38119.215 | | 282.082191 |
| 1511 | 19.418 | 1500.692 | 21493.119 | | 159.049081 |
| 1512 | 21.883 | 1500.677 | 26562.451 | | 196.562137 |
| 1513 | 32.646 | 1500.676 | 267310.469 | | 1978.09747 |
| 1514 | 52.314 | 1499.535 | 113653.813 | | 841.038216 |
| 1515 | 51.11 | 1499.471 | 22515.533 | | 166.614944 |
| 1516 | 31.725 | 1499.319 | 25246.293 | | 186.822568 |
| 1517 | 31.342 | 1498.522 | 205765.641 | | 1522.66574 |
| 1518 | 56.804 | 1497.389 | 8472.941 | | 62.6997634 |
| 1519 | 31.431 | 1495.577 | 37132.027 | | 274.777 |
| 1520 | 25.701 | 1495.51 | 180634.594 | | 1336.696 |
| 1521 | 59.603 | 1495.327 | 12399.307 | | 91.7548718 |
| 1522 | 45.048 | 1494.97 | 42989.801 | | 318.124527 |
| 1523 | 26.474 | 1494.756 | 60827.902 | | 450.126475 |
| 1524 | 56.739 | 1494.4 | 15389.742 | | 113.884091 |
| 1525 | 50.703 | 1494.051 | 49096.602 | | 363.314855 |
| 1526 | 52.323 | 1493.535 | 513625.938 | | 3800.83194 |
| 1527 | 58.794 | 1493.276 | 3180.229 | | 23.5336946 |
| 1528 | 33.034 | 1491.809 | 39573.719 | | 292.845521 |
| 1529 | 22.69 | 1491.715 | 62520.961 | | 462.655111 |
| 1530 | 51.31 | 1490.596 | 64390.566 | | 476.490188 |
| 1531 | 25.699 | 1489.703 | 1324499.13 | | 9801.29353 |
| 1532 | 27.749 | 1489.695 | 35656.855 | | 263.860727 |
| 1533 | 21.937 | 1488.703 | 58306.789 | | 431.470239 |
| 1534 | 26.674 | 1488.673 | 12532.065 | | 92.737281 |
| 1535 | 26.864 | 1488.669 | 9989.638 | | 73.9233212 |
| 1536 | 31.025 | 1488.435 | 115011.352 | | 851.084005 |
| 1537 | 20.064 | 1487.66 | 60555.18 | | 448.108332 |
| 1538 | 57.5 | 1487.261 | 22979.725 | | 170.049965 |
| 1539 | 21.102 | 1486.746 | 86114.773 | | 637.24932 |
| 1540 | 20.302 | 1485.814 | 35362.301 | | 261.681027 |
| 1541 | 19.391 | 1485.744 | 35368.121 | | 261.724095 |
| 1542 | 22.765 | 1485.739 | 325309.406 | | 2407.2896 |
| 1543 | 21.7 | 1485.577 | 179972.781 | | 1331.79858 |
| 1544 | 32.638 | 1484.686 | 284208.563 | | 2103.14337 |
| 1545 | 20.646 | 1483.769 | 99830.844 | | 738.748246 |
| 1546 | 31.547 | 1483.497 | 66147.68 | | 489.492832 |
| 1547 | 21.97 | 1483.484 | 101875.852 | | 753.881305 |
| 1548 | 25.512 | 1482.712 | 47738.512 | | 353.264989 |
| 1549 | 59.952 | 1482.332 | 3723.846 | | 27.5564604 |
| 1550 | 54.947 | 1481.514 | 27755.322 | | 205.389383 |
| 1551 | 51.342 | 1480.523 | 8275.503 | | 61.2387222 |
| 1552 | 59.684 | 1480.179 | 10350.676 | | 76.5950024 |
| 1553 | 19.868 | 1479.653 | 9464.678 | | 70.0386172 |
| 1554 | 51.641 | 1479.497 | 11117.603 | | 82.2702622 |
| 1555 | 32.695 | 1478.703 | 170849.156 | | 1264.28375 |
| 1556 | 56.784 | 1478.422 | 25985.34 | | 192.291516 |
| 1557 | 19.792 | 1477.648 | 12052.142 | | 89.1858508 |
| 1558 | 52.023 | 1477.537 | 506844.188 | | 3750.64699 |
| 1559 | 26.012 | 1476.751 | 22272.973 | | 164.82 |
| 1560 | 25.289 | 1476.723 | 292965.156 | | 2167.94215 |
| 1561 | 31.286 | 1476.502 | 618812.75 | | 4579.21435 |
| 1562 | 50.115 | 1475.578 | 34773.316 | | 257.322538 |
| 1563 | 42.959 | 1475.564 | 14523.597 | | 107.474618 |
| 1564 | 42.656 | 1475.558 | 14509.699 | | 107.371773 |
| 1565 | 18.991 | 1474.936 | 168177.938 | | 1244.51674 |
| 1566 | 59.531 | 1474.323 | 12192.879 | | 90.2273046 |
| 1567 | 45.092 | 1474.032 | 172501.25 | | 1276.50925 |
| 1568 | 25.694 | 1473.735 | 732376.563 | | 5419.58657 |
| 1569 | 22.004 | 1473.732 | 141438.281 | | 1046.64328 |
| 1570 | 57.184 | 1472.278 | 4585.247 | | 33.9308278 |
| 1571 | 58.089 | 1471.285 | 3652.961 | | 27.0319114 |
| 1572 | 21.032 | 1470.737 | 39272.316 | | 290.615138 |
| 1573 | 22.699 | 1469.663 | 479302.844 | | 3546.84105 |
| 1574 | 51.293 | 1469.563 | 30035.428 | | 222.262167 |
| 1575 | 23.445 | 1467.89 | 471461.313 | | 3488.81372 |
| 1576 | 22.953 | 1467.864 | 57678.805 | | 426.823157 |
| 1577 | 22.184 | 1467.716 | 12644.103 | | 93.5663622 |
| 1578 | 21.462 | 1466.901 | 629920.375 | | 4661.41078 |
| 1579 | 25.577 | 1466.726 | 42258.711 | | 312.714461 |
| 1580 | 31.043 | 1466.436 | 690857.25 | | 5112.34365 |
| 1581 | 57.749 | 1466.294 | 25648.756 | | 189.800794 |
| 1582 | 26.832 | 1464.747 | 59844.988 | | 442.852911 |
| 1583 | 52.112 | 1464.549 | 33567.551 | | 248.399877 |
| 1584 | 19.391 | 1463.779 | 92841.156 | | 687.024554 |
| 1585 | 21.836 | 1463.622 | 277416.719 | | 2052.88372 |
| 1586 | 18.809 | 1462.708 | 7145.363 | | 52.8756862 |
| 1587 | 33.265 | 1462.706 | 1376367.75 | | 10185.1214 |
| 1588 | 26.317 | 1461.692 | 7774.391 | | 57.5304934 |
| 1589 | 52.389 | 1461.566 | 83025.867 | | 614.391416 |
| 1590 | 51.105 | 1461.536 | 144334.891 | | 1068.07819 |
| 1591 | 31.735 | 1461.262 | 232331.219 | | 1719.25102 |
| 1592 | 21.435 | 1460.957 | 169996.313 | | 1257.97272 |
| 1593 | 25.385 | 1460.724 | 306236.313 | | 2266.14872 |
| 1594 | 31.326 | 1460.56 | 238888.328 | | 1767.77363 |
| 1595 | 59.746 | 1458.356 | 37428.387 | | 276.970064 |
| 1596 | 25.61 | 1457.725 | 35071.652 | | 259.530225 |
| 1597 | 20.028 | 1455.677 | 29577.705 | | 218.875017 |
| 1598 | 52.304 | 1455.576 | 331660.813 | | 2454.29002 |
| 1599 | 51.028 | 1455.55 | 35265.898 | | 260.967645 |
| 1600 | 60.356 | 1453.339 | 4032.295 | | 29.838983 |
| 1601 | 45.098 | 1453.108 | 32228.975 | | 238.494415 |
| 1602 | 22.717 | 1452.713 | 45172.172 | | 334.274073 |
| 1603 | 20.322 | 1452.661 | 11800.213 | | 87.3215762 |
| 1604 | 25.906 | 1451.759 | 2339633.5 | 20870.565 | 17313.2879 |
| 1605 | 22.074 | 1451.69 | 248246.688 | | 1837.02549 |
| 1606 | 59.326 | 1451.369 | 4813.728 | | 35.6215872 |
| 1607 | 59.701 | 1451.353 | 1536.576 | | 11.3706624 |
| 1608 | 58.268 | 1451.321 | 3431.627 | | 25.3940398 |
| 1609 | 58.798 | 1451.281 | 5105.037 | | 37.7772738 |
| 1610 | 45.048 | 1450.942 | 91047.344 | | 673.750346 |
| 1611 | 23.167 | 1450.662 | 13077.415 | | 96.772871 |
| 1612 | 31.043 | 1450.47 | 449713.969 | | 3327.88337 |
| 1613 | 21.432 | 1449.717 | 434321.813 | | 3213.98142 |
| 1614 | 22.084 | 1448.686 | 19411.707 | | 143.646632 |
| 1615 | 19.402 | 1447.709 | 167506.141 | | 1239.54544 |
| 1616 | 21.69 | 1447.618 | 597990.688 | | 4425.13109 |
| 1617 | 42.128 | 1447.568 | 8425.077 | | 62.3455698 |
| 1618 | 59.775 | 1447.348 | 5009.105 | | 37.067377 |
| 1619 | 32.688 | 1446.725 | 572850 | | 4239.09 |
| 1620 | 54.385 | 1446.459 | 14195.906 | | 105.049704 |
| 1621 | 21.355 | 1445.637 | 101465.133 | | 750.841984 |
| 1622 | 31.582 | 1445.631 | 231467.766 | | 1712.86147 |
| 1623 | 51.134 | 1445.524 | 69437.68 | | 513.838832 |
| 1624 | 52.726 | 1445.505 | 4868.882 | | 36.0297268 |
| 1625 | 25.539 | 1444.755 | 80478.234 | | 595.538932 |
| 1626 | 20.78 | 1444.136 | 11996.748 | | 88.7759352 |
| 1627 | 20.099 | 1443.858 | 48822.273 | | 361.28482 |
| 1628 | 32.312 | 1443.65 | 21127.52 | | 156.343648 |
| 1629 | 41.941 | 1443.593 | 224228.266 | | 1659.28917 |
| 1630 | 47.591 | 1442.561 | 5376.899 | | 39.7890526 |
| 1631 | 59.774 | 1442.484 | 29060.875 | | 215.050475 |
| 1632 | 52.084 | 1442.129 | 9874.53 | | 73.071522 |
| 1633 | 56.749 | 1440.464 | 17626.166 | | 130.433628 |
| 1634 | 53.231 | 1440.327 | 7973.153 | | 59.0013322 |
| 1635 | 51.285 | 1439.505 | 721402.5 | | 5338.3785 |
| 1636 | 30.909 | 1439.44 | 47137.531 | | 348.817729 |
| 1637 | 25.302 | 1438.762 | 476602.969 | | 3526.86197 |
| 1638 | 26.098 | 1438.714 | 131795.766 | | 975.288668 |
| 1639 | 31.282 | 1438.543 | 532467.688 | | 3940.26089 |
| 1640 | 59.661 | 1436.368 | 26654.971 | | 197.246785 |
| 1641 | 25.623 | 1435.749 | 403066.875 | 5145.30946 | 2982.69488 |
| 1642 | 57.527 | 1434.335 | 61294.555 | | 453.579707 |
| 1643 | 19.291 | 1430.597 | 19614.697 | | 145.148758 |
| 1644 | 45.07 | 1430.006 | 183551.078 | | 1358.27798 |
| 1645 | 31.217 | 1428.461 | 2081349.38 | | 15401.9854 |
| 1646 | 41.061 | 1427.616 | 25458.787 | | 188.395024 |
| 1647 | 21.887 | 1425.708 | 515758.594 | | 3816.6136 |
| 1648 | 32.632 | 1424.748 | 292768.469 | | 2166.48667 |
| 1649 | 20.188 | 1423.596 | 59391.871 | | 439.499845 |
| 1650 | 51.159 | 1423.54 | 678686.125 | | 5022.27733 |
| 1651 | 19.388 | 1423.463 | 14447.822 | | 106.913883 |
| 1652 | 60.406 | 1423.454 | 6015.27 | | 44.512998 |
| 1653 | 21.462 | 1423.091 | 689747.75 | | 5104.13335 |
| 1654 | 31.762 | 1423.015 | 158091.891 | | 1169.87999 |
| 1655 | 25.58 | 1422.755 | 154930.813 | | 1146.48802 |
| 1656 | 59.962 | 1420.822 | 60185.059 | | 445.369437 |
| 1657 | 47.513 | 1420.228 | 39887.438 | | 295.167041 |
| 1658 | 25.381 | 1419.602 | 160612.406 | | 1188.5318 |
| 1659 | 22.881 | 1419.568 | 164075.094 | | 1214.1557 |
| 1660 | 59.173 | 1418.341 | 8607.118 | | 63.6926732 |
| 1661 | 25.701 | 1416.669 | 250310.063 | | 1852.29447 |
| 1662 | 19.933 | 1415.938 | 15313.807 | | 113.322172 |
| 1663 | 59.853 | 1415.354 | 2729.604 | | 20.1990696 |
| 1664 | 60.002 | 1414.327 | 2912.038 | | 21.5490812 |
| 1665 | 25.344 | 1413.599 | 40637.359 | | 300.716457 |
| 1666 | 23.791 | 1413.597 | 50547.738 | | 374.053261 |
| 1667 | 45.052 | 1412.986 | 13919.365 | | 103.003301 |
| 1668 | 25.335 | 1412.84 | 14382.029 | | 106.427015 |
| 1669 | 32.132 | 1412.554 | 54933.371 | | 406.506945 |
| 1670 | 31.045 | 1412.512 | 527367.25 | | 3902.51765 |
| 1671 | 57.556 | 1412.347 | 25780.541 | | 190.776003 |
| 1672 | 32.409 | 1411.64 | 16508.381 | | 122.162019 |
| 1673 | 59.681 | 1410.329 | 5465.604 | | 40.4454696 |
| 1674 | 31.527 | 1407.728 | 41320.148 | | 305.769095 |
| 1675 | 21.351 | 1407.672 | 178818.625 | | 1323.25783 |
| 1676 | 51.17 | 1407.562 | 118187.75 | | 874.58935 |
| 1677 | 52.677 | 1407.503 | 40964.031 | | 303.133829 |
| 1678 | 45.069 | 1406.92 | 71280.117 | | 527.472866 |
| 1679 | 59.199 | 1406.339 | 3843.809 | | 28.4441866 |
| 1680 | 60.013 | 1406.165 | 13561.318 | | 100.353753 |
| 1681 | 22.464 | 1405.749 | 23630.906 | | 174.868704 |
| 1682 | 20.155 | 1405.702 | 79460.164 | | 588.005214 |
| 1683 | 32.604 | 1405.687 | 482839.156 | | 3573.00975 |
| 1684 | 41.281 | 1405.646 | 101633.367 | | 752.086916 |
| 1685 | 59.693 | 1405.026 | 18004.275 | | 133.231635 |
| 1686 | 25.352 | 1403.563 | 92161.961 | | 681.998511 |
| 1687 | 53.201 | 1402.099 | 23272.326 | | 172.215212 |
| 1688 | 51.072 | 1401.522 | 388249.438 | | 2873.04584 |
| 1689 | 31.654 | 1401.509 | 7511.224 | | 55.5830576 |
| 1690 | 30.98 | 1401.473 | 171155.313 | | 1266.54932 |
| 1691 | 25.705 | 1400.678 | 71240.352 | | 527.178605 |
| 1692 | 60.454 | 1399.435 | 15121.45 | | 111.89873 |
| 1693 | 19.068 | 1397.692 | 3839.199 | | 28.4100726 |
| 1694 | 25.389 | 1397.57 | 528235.875 | | 3908.94548 |
| 1695 | 32.288 | 1396.606 | 10441.645 | | 77.268173 |
| 1696 | 31.138 | 1396.434 | 127553.898 | | 943.898845 |
| 1697 | 57.725 | 1396.36 | 55281.977 | | 409.08663 |
| 1698 | 21.421 | 1392.695 | 709464.313 | | 5250.03592 |
| 1699 | 59.374 | 1391.448 | 4601.684 | | 34.0524616 |
| 1700 | 59.651 | 1391.428 | 2721.883 | | 20.1419342 |
| 1701 | 45.062 | 1390.986 | 37551.5 | | 277.8811 |
| 1702 | 31.091 | 1390.532 | 1131354.75 | | 8372.02515 |
| 1703 | 32.449 | 1389.93 | 406237.781 | | 3006.15958 |
| 1704 | 43.567 | 1388.482 | 94132.688 | | 696.581891 |
| 1705 | 51.546 | 1386.784 | 3704.049 | | 27.4099626 |
| 1706 | 25.04 | 1386.521 | 93427.875 | | 691.366275 |
| 1707 | 59.273 | 1386.36 | 24964.27 | | 184.735598 |
| 1708 | 45.063 | 1385.978 | 210238.953 | | 1555.76825 |
| 1709 | 51.138 | 1385.566 | 41882.465 | | 309.930241 |
| 1710 | 30.901 | 1385.488 | 20099.48 | | 148.736152 |
| 1711 | 59.7 | 1382.403 | 30916.771 | | 228.784105 |
| 1712 | 47.077 | 1382.273 | 64972.203 | | 480.794302 |
| 1713 | 25.349 | 1381.698 | 426013.25 | | 3152.49805 |
| 1714 | 21.186 | 1380.693 | 20415.264 | | 151.072954 |
| 1715 | 25.109 | 1380.547 | 10967.781 | | 81.1615794 |
| 1716 | 53.108 | 1380.342 | 13301.506 | | 98.4311444 |
| 1717 | 25.679 | 1378.704 | 342898.781 | 2415.39444 | 2537.45098 |
| 1718 | 49.632 | 1378.394 | 5379.274 | | 39.8066276 |
| 1719 | 18.97 | 1377.85 | 30348.645 | | 224.579973 |
| 1720 | 25.346 | 1375.624 | 46687.473 | | 345.4873 |
| 1721 | 32.125 | 1375.532 | 67528.18 | | 499.708532 |
| 1722 | 37.369 | 1375.445 | 8201.093 | | 60.6880882 |
| 1723 | 34.801 | 1375.438 | 17825.877 | | 131.91149 |
| 1724 | 55.31 | 1374.42 | 31385.559 | | 232.253137 |
| 1725 | 56.785 | 1374.415 | 9927.623 | | 73.4644102 |
| 1726 | 32.405 | 1373.689 | 38635.266 | | 285.900968 |
| 1727 | 60.227 | 1373.36 | 6000.033 | | 44.4002442 |
| 1728 | 45.05 | 1368.949 | 34093.426 | | 252.291352 |
| 1729 | 32.464 | 1367.728 | 132403.016 | | 979.782318 |
| 1730 | 55.22 | 1367.496 | 1103.651 | | 8.1670174 |
| 1731 | 60.375 | 1366.465 | 12431.157 | | 91.9905618 |
| 1732 | 25.359 | 1365.647 | 47731.473 | | 353.2129 |
| 1733 | 45.052 | 1365.037 | 104198.375 | | 771.067975 |
| 1734 | 19.189 | 1364.748 | 11513.005 | | 85.196237 |
| 1735 | 25.813 | 1364.605 | 75452.961 | | 558.351911 |
| 1736 | 25.024 | 1364.429 | 298666.156 | | 2210.12955 |
| 1737 | 51.066 | 1363.59 | 4498.999 | | 33.2925926 |
| 1738 | 30.979 | 1363.458 | 95695.992 | | 708.150341 |
| 1739 | 45.02 | 1362.89 | 110947.891 | | 821.014393 |
| 1740 | 32.068 | 1359.662 | 52750.859 | | 390.356357 |
| 1741 | 25.534 | 1359.656 | 1906445.75 | | 14107.6986 |
| 1742 | 31.049 | 1358.466 | 151038.281 | | 1117.68328 |
| 1743 | 49.608 | 1356.381 | 45604.996 | | 337.47697 |
| 1744 | 59.708 | 1356.319 | 29863.813 | | 220.992216 |
| 1745 | 60.659 | 1355.61 | 1210.575 | | 8.958255 |
| 1746 | 55.572 | 1354.558 | 1049.276 | | 7.7646424 |
| 1747 | 58.894 | 1354.391 | 2738.662 | | 20.2660988 |
| 1748 | 23.999 | 1353.72 | 101441.016 | | 750.663518 |
| 1749 | 23.416 | 1352.847 | 43856.227 | | 324.53608 |
| 1750 | 30.605 | 1352.398 | 90038.555 | | 666.285307 |
| 1751 | 32.39 | 1351.686 | 32230.543 | | 238.506018 |
| 1752 | 34.799 | 1351.387 | 8059.731 | | 59.6420094 |
| 1753 | 25.989 | 1350.605 | 40075.652 | | 296.559825 |
| 1754 | 59.605 | 1350.549 | 6613.854 | | 48.9425196 |
| 1755 | 59.372 | 1349.363 | 5556.154 | | 41.1155396 |
| 1756 | 25.073 | 1348.535 | 290868.375 | | 2152.42598 |
| 1757 | 18.811 | 1347.718 | 12626.824 | | 93.4384976 |
| 1758 | 25.458 | 1343.664 | 766104.375 | | 5669.17238 |
| 1759 | 26.465 | 1342.628 | 17343.17 | | 128.339458 |
| 1760 | 53.156 | 1342.392 | 5754.031 | | 42.5798294 |
| 1761 | 45.041 | 1341.946 | 191931.266 | | 1420.29137 |
| 1762 | 29.936 | 1339.384 | 45313.438 | | 335.319441 |
| 1763 | 22.748 | 1338.66 | 77636.578 | | 574.510677 |
| 1764 | 60.353 | 1338.395 | 2928.564 | | 21.6713736 |
| 1765 | 30.621 | 1336.417 | 37646.219 | | 278.582021 |
| 1766 | 55.091 | 1334.981 | 17385.801 | | 128.654927 |
| 1767 | 59.445 | 1334.35 | 36656.449 | | 271.257723 |
| 1768 | 19.066 | 1332.751 | 11472.639 | | 84.8975286 |
| 1769 | 25.099 | 1332.551 | 113287.906 | | 838.330504 |
| 1770 | 59.549 | 1330.309 | 2890.745 | | 21.391513 |
| 1771 | 59.275 | 1330.277 | 9237.706 | | 68.3590244 |
| 1772 | 25.154 | 1326.551 | 949441.625 | | 7025.86803 |
| 1773 | 45.045 | 1324.926 | 42727.93 | | 316.186682 |
| 1774 | 31.961 | 1322.603 | 28099.598 | | 207.937025 |
| 1775 | 25.586 | 1321.685 | 2478338.5 | | 18339.7049 |
| 1776 | 30.808 | 1321.321 | 58397.359 | | 432.140457 |
| 1777 | 45.004 | 1321.021 | 66336.734 | | 490.891832 |
| 1778 | 20.636 | 1320.662 | 25817.596 | | 191.05021 |
| 1779 | 32.429 | 1319.441 | 11738.492 | | 86.8648408 |
| 1780 | 31.4 | 1319.42 | 6886.302 | | 50.9586348 |
| 1781 | 52.954 | 1319.408 | 3111.002 | | 23.0214148 |
| 1782 | 50.24 | 1319.394 | 4092.932 | | 30.2876968 |
| 1783 | 48.515 | 1319.389 | 12137.362 | | 89.8164788 |
| 1784 | 34.136 | 1319.387 | 43983.848 | | 325.480475 |
| 1785 | 35.227 | 1319.362 | 4880.322 | | 36.1143828 |
| 1786 | 45 | 1318.861 | 87233.422 | | 645.527323 |
| 1787 | 31.907 | 1318.611 | 13398.739 | | 99.1506686 |
| 1788 | 41.768 | 1318.589 | 232563.891 | | 1720.97279 |
| 1789 | 39.529 | 1318.407 | 9566.113 | | 70.7892362 |
| 1790 | 46.954 | 1318.405 | 13212.546 | | 97.7728404 |
| 1791 | 53.154 | 1318.387 | 4069.485 | | 30.114189 |
| 1792 | 39.074 | 1318.375 | 7447.503 | | 55.1115222 |
| 1793 | 59.704 | 1318.363 | 36163.148 | | 267.607295 |
| 1794 | 30.603 | 1314.452 | 239448.859 | | 1771.92156 |
| 1795 | 21.917 | 1312.679 | 77039.18 | | 570.089932 |
| 1796 | 26.005 | 1312.635 | 141686.484 | | 1048.47998 |
| 1797 | 52.047 | 1311.498 | 829.617 | | 6.1391658 |
| 1798 | 25.103 | 1310.563 | 660235.938 | | 4885.74594 |
| 1799 | 60.227 | 1306.401 | 3903.396 | | 28.8851304 |
| 1800 | 45.07 | 1303.966 | 34127.25 | | 252.54165 |
| 1801 | 25.985 | 1302.62 | 19980.832 | | 147.858157 |
| 1802 | 51.644 | 1302.438 | 2354.193 | | 17.4210282 |
| 1803 | 59.746 | 1302.393 | 45806.871 | | 338.970845 |
| 1804 | 22.252 | 1301.272 | 57392.363 | | 424.703486 |
| 1805 | 54.153 | 1300.353 | 8612.625 | | 63.733425 |
| 1806 | 21.95 | 1299.873 | 83524.633 | | 618.082284 |
| 1807 | 51.573 | 1299.424 | 30573.758 | | 226.245809 |
| 1808 | 53.107 | 1299.416 | 25302.227 | | 187.23648 |
| 1809 | 19.258 | 1298.642 | 21156.547 | | 156.558448 |
| 1810 | 59.374 | 1298.359 | 12482.001 | | 92.3668074 |
| 1811 | 45.03 | 1297.919 | 185944.781 | | 1375.99138 |
| 1812 | 19.022 | 1296.685 | 28328.535 | | 209.631159 |
| 1813 | 59.676 | 1295.779 | 9187.956 | | 67.9908744 |
| 1814 | 28.449 | 1295.485 | 170789.578 | | 1263.84288 |
| 1815 | 25.008 | 1294.604 | 46179.98 | | 341.731852 |
| 1816 | 30.249 | 1293.53 | 103213.75 | | 763.78175 |
| 1817 | 23.194 | 1292.502 | 19937.287 | | 147.535924 |
| 1818 | 30.646 | 1290.433 | 8808.077 | | 65.1797698 |
| 1819 | 28.43 | 1289.456 | 18305.74 | | 135.462476 |
| 1820 | 58.946 | 1289.403 | 1384.637 | | 10.2463138 |
| 1821 | 59.627 | 1289.387 | 2301.153 | | 17.0285322 |
| 1822 | 19.693 | 1289.386 | 4377.44 | | 32.393056 |
| 1823 | 25.244 | 1288.534 | 2999592.25 | | 22196.9827 |
| 1824 | 59.353 | 1286.338 | 15882.02 | | 117.526948 |
| 1825 | 30.82 | 1283.472 | 39585.328 | | 292.931427 |
| 1826 | 51.523 | 1283.445 | 6039.294 | | 44.6907756 |
| 1827 | 53.186 | 1283.43 | 8806.266 | | 65.1663684 |
| 1828 | 34.802 | 1283.335 | 8705.754 | | 64.4225796 |
| 1829 | 59.03 | 1282.394 | 15341.116 | | 113.524258 |
| 1830 | 55.529 | 1282.378 | 7111.861 | | 52.6277714 |
| 1831 | 45.022 | 1280.883 | 30969.486 | | 229.174196 |
| 1832 | 19.518 | 1280.665 | 11716.79 | | 86.704246 |
| 1833 | 59.668 | 1280.411 | 49052 | | 362.9848 |
| 1834 | 28.452 | 1279.513 | 51832.395 | | 383.559723 |
| 1835 | 51.44 | 1277.454 | 82467.625 | | 610.260425 |
| 1836 | 52.927 | 1277.446 | 43342.348 | | 320.733375 |
| 1837 | 45.003 | 1276.982 | 70758.93 | | 523.616082 |
| 1838 | 30.44 | 1276.942 | 232610.75 | | 1721.31955 |
| 1839 | 50.608 | 1276.907 | 1109.392 | | 8.2095008 |
| 1840 | 23.116 | 1276.515 | 122081.953 | | 903.406452 |
| 1841 | 44.995 | 1274.825 | 81134.063 | | 600.392066 |
| 1842 | 26.295 | 1272.61 | 41668.891 | | 308.349793 |
| 1843 | 25.135 | 1272.604 | 1010380.88 | | 7476.81848 |
| 1844 | 20.731 | 1272.48 | 34951.859 | | 258.643757 |
| 1845 | 30.456 | 1272.332 | 289698.906 | | 2143.7719 |
| 1846 | 31.95 | 1270.569 | 58033.617 | | 429.448766 |
| 1847 | 54.813 | 1270.498 | 1479.774 | | 10.9503276 |
| 1848 | 59.96 | 1270.475 | 3445.866 | | 25.4994084 |
| 1849 | 57.989 | 1270.447 | 1281.669 | | 9.4843506 |
| 1850 | 59.188 | 1270.424 | 5638.458 | | 41.7245892 |
| 1851 | 37.091 | 1269.475 | 1353.805 | | 10.018157 |
| 1852 | 40.97 | 1269.437 | 2427.428 | | 17.9629672 |
| 1853 | 35.834 | 1269.418 | 2728.887 | | 20.1937638 |
| 1854 | 40.087 | 1269.413 | 1651.728 | | 12.2227872 |
| 1855 | 39.127 | 1269.409 | 5886.622 | | 43.5610028 |
| 1856 | 36.13 | 1269.399 | 4146.72 | | 30.685728 |
| 1857 | 30.571 | 1267.515 | 21216.277 | | 157.00045 |
| 1858 | 59.349 | 1266.399 | 6219.467 | | 46.0240558 |
| 1859 | 54.894 | 1266.362 | 4477.317 | | 33.1321458 |
| 1860 | 22.184 | 1263.606 | 94234.227 | | 697.33328 |
| 1861 | 59.935 | 1263.437 | 57252.406 | | 423.667804 |
| 1862 | 34.938 | 1263.312 | 16850.229 | | 124.691695 |
| 1863 | 37.489 | 1262.942 | 10585.427 | | 78.3321598 |
| 1864 | 55.342 | 1262.696 | 14468.394 | | 107.066116 |
| 1865 | 52.525 | 1262.35 | 8346.335 | | 61.762879 |
| 1866 | 39.287 | 1262.338 | 12846.079 | | 95.0609846 |
| 1867 | 40.214 | 1262.335 | 8975.618 | | 66.4195732 |
| 1868 | 58.04 | 1262.315 | 3522.354 | | 26.0654196 |
| 1869 | 21.765 | 1262.289 | 123903.367 | | 916.884916 |
| 1870 | 51.525 | 1261.458 | 99836.336 | | 738.788886 |
| 1871 | 53.043 | 1261.455 | 83416.992 | | 617.285741 |
| 1872 | 23.116 | 1260.537 | 46480.016 | | 343.952118 |
| 1873 | 34.799 | 1259.311 | 14362.161 | | 106.279991 |
| 1874 | 28.459 | 1257.534 | 298317.219 | | 2207.54742 |
| 1875 | 31.559 | 1255.699 | 116446.039 | | 861.700689 |
| 1876 | 30.3 | 1255.583 | 846248.25 | | 6262.23705 |
| 1877 | 32.837 | 1255.567 | 35492.637 | | 262.645514 |
| 1878 | 33.291 | 1255.561 | 28837.1 | | 213.39454 |
| 1879 | 51.244 | 1255.489 | 2861.904 | | 21.1780896 |
| 1880 | 32.024 | 1254.973 | 95395.18 | | 705.924332 |
| 1881 | 23.278 | 1254.568 | 121895.391 | | 902.025893 |
| 1882 | 59.74 | 1254.366 | 5949.095 | | 44.023303 |
| 1883 | 57.787 | 1254.364 | 4684.266 | | 34.6635684 |
| 1884 | 58.949 | 1254.347 | 2524.573 | | 18.6818402 |
| 1885 | 57.081 | 1254.319 | 8885.642 | | 65.7537508 |
| 1886 | 55.421 | 1254.301 | 4541.622 | | 33.6080028 |
| 1887 | 45.012 | 1253.896 | 147699.641 | | 1092.97734 |
| 1888 | 28.407 | 1251.496 | 141158.859 | | 1044.57556 |
| 1889 | 25.249 | 1250.631 | 3974873.75 | 34371.53 | 29414.0658 |
| 1890 | 58.643 | 1250.455 | 11072.911 | | 81.9395414 |
| 1891 | 18.98 | 1249.756 | 27491.875 | | 203.439875 |
| 1892 | 31.656 | 1248.538 | 26623.307 | | 197.012472 |
| 1893 | 32.92 | 1247.361 | 41003.309 | | 303.424487 |
| 1894 | 51.498 | 1245.498 | 8761.765 | | 64.837061 |
| 1895 | 53.085 | 1245.476 | 44366.938 | | 328.315341 |
| 1896 | 19.632 | 1244.64 | 30831.113 | | 228.150236 |
| 1897 | 59.714 | 1242.427 | 56404.984 | | 417.396882 |
| 1898 | 56.779 | 1241.078 | 2758.177 | | 20.4105098 |
| 1899 | 30.278 | 1239.607 | 317820.063 | | 2351.86847 |
| 1900 | 31.547 | 1239.563 | 59098.148 | | 437.326295 |
| 1901 | 28.472 | 1239.526 | 69276.641 | | 512.647143 |
| 1902 | 51.321 | 1239.492 | 69059.977 | | 511.04383 |
| 1903 | 52.866 | 1239.474 | 32897.426 | | 243.440952 |
| 1904 | 60.102 | 1238.604 | 6424.091 | | 47.5382734 |
| 1905 | 23.198 | 1238.572 | 492393.188 | | 3643.70959 |
| 1906 | 25.057 | 1238.532 | 9391.734 | | 69.4988316 |
| 1907 | 37.49 | 1237.332 | 6531.968 | | 48.3365632 |
| 1908 | 45.022 | 1236.87 | 46682.961 | | 345.453911 |
| 1909 | 19.717 | 1236.611 | 7412.524 | | 54.8526776 |
| 1910 | 40.236 | 1236.283 | 8209.991 | | 60.7539334 |
| 1911 | 52.172 | 1235.898 | 888.905 | | 6.577897 |
| 1912 | 19.301 | 1235.868 | 4725.906 | | 34.9717044 |
| 1913 | 30.417 | 1234.37 | 856949.25 | | 6341.42445 |
| 1914 | 31.611 | 1233.532 | 122064.055 | | 903.274007 |
| 1915 | 25.133 | 1232.595 | 24227.906 | | 179.286504 |
| 1916 | 21.214 | 1231.482 | 60734.938 | | 449.438541 |
| 1917 | 32.839 | 1231.084 | 60950.719 | | 451.035321 |
| 1918 | 42.855 | 1230.887 | 154045.125 | | 1139.93393 |
| 1919 | 30.565 | 1229.563 | 91573.625 | | 677.644825 |
| 1920 | 22.209 | 1228.627 | 18700.881 | | 138.386519 |
| 1921 | 60.379 | 1227.376 | 3367.176 | | 24.9171024 |
| 1922 | 52.522 | 1227.324 | 6150.93 | | 45.516882 |
| 1923 | 51.79 | 1227.323 | 4718.815 | | 34.919231 |
| 1924 | 49.813 | 1227.305 | 11513.576 | | 85.2004624 |
| 1925 | 40.192 | 1227.301 | 7194.075 | | 53.236155 |
| 1926 | 59.636 | 1226.901 | 24848.395 | | 183.878123 |
| 1927 | 21.859 | 1224.549 | 44181.957 | | 326.946482 |
| 1928 | 30.406 | 1224.415 | 69779.727 | | 516.36998 |
| 1929 | 23.094 | 1222.581 | 91712.016 | | 678.668918 |
| 1930 | 51.595 | 1222.471 | 3662.568 | | 27.1030032 |
| 1931 | 30.29 | 1218.52 | 476128.844 | | 3523.35345 |
| 1932 | 31.528 | 1217.669 | 163110.922 | | 1207.02082 |
| 1933 | 25.119 | 1217.539 | 169168.734 | | 1251.84863 |
| 1934 | 23.259 | 1216.597 | 263113.625 | | 1947.04083 |
| 1935 | 44.996 | 1215.355 | 16854.1 | | 124.72034 |
| 1936 | 31.638 | 1211.447 | 117543.125 | | 869.819125 |
| 1937 | 55.57 | 1210.742 | 2894.646 | | 21.4203804 |
| 1938 | 20.68 | 1210.615 | 33522.359 | | 248.065457 |
| 1939 | 31.023 | 1210.478 | 47086.57 | | 348.440618 |
| 1940 | 60.002 | 1210.378 | 2460.045 | | 18.204333 |
| 1941 | 59.452 | 1210.36 | 2822.82 | | 20.888868 |
| 1942 | 53.367 | 1210.321 | 12792.264 | | 94.6627536 |
| 1943 | 56.782 | 1210.2 | 4122.384 | | 30.5056416 |
| 1944 | 45.004 | 1209.865 | 121766.766 | | 901.074068 |
| 1945 | 25.804 | 1209.47 | 31311.213 | | 231.702976 |
| 1946 | 20.729 | 1206.596 | 10410.285 | | 77.036109 |
| 1947 | 51.214 | 1205.491 | 3944.53 | | 29.189522 |
| 1948 | 31.553 | 1203.616 | 23597.301 | | 174.620027 |
| 1949 | 49.516 | 1203.133 | 8821.522 | | 65.2792628 |
| 1950 | 30.195 | 1202.589 | 39710.629 | | 293.858655 |
| 1951 | 50.689 | 1202.365 | 25647.943 | | 189.794778 |
| 1952 | 51.135 | 1201.518 | 6787.769 | | 50.2294906 |
| 1953 | 42.67 | 1200.783 | 72117.422 | | 533.668923 |
| 1954 | 23.167 | 1200.609 | 1313535.88 | | 9720.16548 |
| 1955 | 25.206 | 1200.547 | 16658.26 | | 123.271124 |
| 1956 | 46.71 | 1200.503 | 19274.873 | | 142.63406 |
| 1957 | 30.044 | 1198.562 | 22444.525 | | 166.089485 |
| 1958 | 59.87 | 1198.361 | 4803.752 | | 35.5477648 |
| 1959 | 59.432 | 1198.353 | 4145.694 | | 30.6781356 |
| 1960 | 19.706 | 1198.222 | 12631.365 | | 93.472101 |
| 1961 | 21.013 | 1197.031 | 87272.023 | | 645.81297 |
| 1962 | 30.406 | 1196.417 | 620780.5 | | 4593.7757 |
| 1963 | 60.6 | 1196.352 | 7650.589 | | 56.6143586 |
| 1964 | 31.578 | 1195.579 | 464075.188 | | 3434.15639 |
| 1965 | 59.222 | 1190.363 | 5847.735 | | 43.273239 |
| 1966 | 53.252 | 1188.334 | 55063.84 | | 407.472416 |
| 1967 | 59.825 | 1187.418 | 3255.71 | | 24.092254 |
| 1968 | 21.882 | 1186.597 | 192521.25 | | 1424.65725 |
| 1969 | 60.255 | 1186.382 | 6004.868 | | 44.4360232 |
| 1970 | 30.174 | 1185.657 | 58927.867 | | 436.066216 |
| 1971 | 42.453 | 1184.524 | 53516.508 | | 396.022159 |
| 1972 | 59.174 | 1184.402 | 4914.623 | | 36.3682102 |
| 1973 | 21.325 | 1183.635 | 234638.141 | | 1736.32224 |
| 1974 | 59.7 | 1183.443 | 7250.531 | | 53.6539294 |
| 1975 | 31.801 | 1182.586 | 11399.009 | | 84.3526666 |
| 1976 | 31.299 | 1181.569 | 94998.031 | | 702.985429 |
| 1977 | 49.745 | 1181.254 | 3827.925 | | 28.326645 |
| 1978 | 56.778 | 1181.221 | 3641.417 | | 26.9464858 |
| 1979 | 60.62 | 1180.368 | 5690.042 | | 42.1063108 |
| 1980 | 50.733 | 1180.335 | 38225.785 | | 282.870809 |
| 1981 | 31.583 | 1179.597 | 236420.203 | | 1749.5095 |
| 1982 | 19.143 | 1178.579 | 11057.592 | | 81.8261808 |
| 1983 | 20.779 | 1178.495 | 131686.141 | | 974.477443 |
| 1984 | 51.247 | 1178.431 | 6476.849 | | 47.9286826 |
| 1985 | 41.654 | 1178.397 | 6623.212 | | 49.0117688 |
| 1986 | 56.782 | 1178.216 | 4255.032 | | 31.4872368 |
| 1987 | 25.333 | 1175.511 | 22551.197 | | 166.878858 |
| 1988 | 24.101 | 1172.463 | 51734.363 | | 382.834286 |
| 1989 | 53.214 | 1172.348 | 38737.391 | | 286.656693 |
| 1990 | 44.949 | 1170.829 | 10637.769 | | 78.7194906 |
| 1991 | 22.67 | 1169.26 | 18541.186 | | 137.204776 |
| 1992 | 54.977 | 1169.167 | 7155.165 | | 52.948221 |
| 1993 | 60.253 | 1166.411 | 2237.358 | | 16.5564492 |
| 1994 | 53.22 | 1166.339 | 140311.391 | | 1038.30429 |
| 1995 | 31.322 | 1165.301 | 58614.336 | | 433.746086 |
| 1996 | 31.542 | 1163.587 | 34373.684 | | 254.365262 |
| 1997 | 42.864 | 1163.561 | 317640.313 | | 2350.53832 |
| 1998 | 20.221 | 1162.602 | 72301.289 | | 535.029539 |
| 1999 | 30.294 | 1160.457 | 18931.172 | | 140.090673 |
| 2000 | 20.533 | 1159.644 | 11926.854 | | 88.2587196 |
| 2001 | 25.361 | 1158.633 | 29713.969 | | 219.883371 |
| 2002 | 31.529 | 1157.615 | 44626.973 | | 330.2396 |
| 2003 | 24.123 | 1156.492 | 33221.211 | | 245.836961 |
| 2004 | 19.431 | 1154.638 | 47105.656 | | 348.581854 |
| 2005 | 31.311 | 1154.523 | 72400.438 | | 535.763241 |
| 2006 | 23.924 | 1152.527 | 201380.953 | | 1490.21905 |
| 2007 | 30.384 | 1152.393 | 58578.801 | | 433.483127 |
| 2008 | 21.985 | 1150.625 | 37468.707 | | 277.268432 |
| 2009 | 53.194 | 1150.361 | 120706.516 | | 893.228218 |
| 2010 | 30.027 | 1149.312 | 119640.453 | | 885.339352 |
| 2011 | 42.541 | 1147.575 | 30441.695 | | 225.268543 |
| 2012 | 23.629 | 1144.649 | 29225.717 | | 216.270306 |
| 2013 | 31.311 | 1143.136 | 23237.896 | | 171.96043 |
| 2014 | 24.001 | 1142.545 | 23198.781 | | 171.670979 |
| 2015 | 50.597 | 1142.373 | 118697.242 | | 878.359591 |
| 2016 | 23.275 | 1141.59 | 47004.074 | | 347.830148 |
| 2017 | 21.014 | 1140.488 | 174479.281 | | 1291.14668 |
| 2018 | 41.649 | 1140.415 | 5001.429 | | 37.0105746 |
| 2019 | 19.442 | 1138.647 | 71437.797 | | 528.639698 |
| 2020 | 31.367 | 1138.553 | 111894.984 | | 828.022882 |
| 2021 | 25.359 | 1137.454 | 68654.234 | | 508.041332 |
| 2022 | 53.625 | 1137.364 | 4344.629 | | 32.1502546 |
| 2023 | 23.802 | 1136.534 | 21588.393 | | 159.754108 |
| 2024 | 25.107 | 1135.542 | 37640.664 | | 278.540914 |
| 2025 | 22.904 | 1134.648 | 71461.75 | | 528.81695 |
| 2026 | 24.113 | 1134.504 | 347155.5 | | 2568.9507 |
| 2027 | 60.091 | 1134.459 | 5499.415 | | 40.695671 |
| 2028 | 59.353 | 1134.437 | 3032.865 | | 22.443201 |
| 2029 | 53.188 | 1134.391 | 56996.746 | | 421.77592 |
| 2030 | 51.575 | 1134.21 | 9499.726 | | 70.2979724 |
| 2031 | 29.971 | 1133.34 | 41626.363 | | 308.035086 |
| 2032 | 20.936 | 1128.605 | 47002.93 | | 347.821682 |
| 2033 | 28.199 | 1128.485 | 105328.227 | | 779.42888 |
| 2034 | 53.209 | 1128.38 | 354067.813 | | 2620.10182 |
| 2035 | 50.589 | 1126.389 | 28851.957 | | 213.504482 |
| 2036 | 31.552 | 1125.231 | 11812.239 | | 87.4105686 |
| 2037 | 41.868 | 1125.221 | 15596.393 | | 115.413308 |
| 2038 | 32.903 | 1125.218 | 126022.156 | | 932.563954 |
| 2039 | 31.908 | 1125.217 | 25750.453 | | 190.553352 |
| 2040 | 39.864 | 1125.21 | 6728.264 | | 49.7891536 |
| 2041 | 36.571 | 1125.206 | 22293.379 | | 164.971005 |
| 2042 | 39.556 | 1125.202 | 16396.361 | | 121.333071 |
| 2043 | 26.016 | 1125.161 | 31188 | | 230.7912 |
| 2044 | 29.167 | 1125.148 | 41243.914 | | 305.204964 |
| 2045 | 38.059 | 1124.829 | 29475.412 | | 218.118049 |
| 2046 | 31.341 | 1122.552 | 34561.762 | | 255.757039 |
| 2047 | 23.72 | 1122.456 | 36783.59 | | 272.198566 |
| 2048 | 44.964 | 1121.808 | 44746.047 | | 331.120748 |
| 2049 | 18.812 | 1119.758 | 5198.635 | | 38.469899 |
| 2050 | 24.134 | 1118.525 | 119093.102 | | 881.288955 |
| 2051 | 25.151 | 1118.499 | 30842.926 | | 228.237652 |
| 2052 | 23.899 | 1114.554 | 208412.047 | | 1542.24915 |
| 2053 | 29.989 | 1111.346 | 183555.891 | | 1358.31359 |
| 2054 | 28.181 | 1110.479 | 70840.586 | | 524.220336 |
| 2055 | 51.147 | 1110.414 | 125425.094 | | 928.145696 |
| 2056 | 20.151 | 1108.578 | 25817.43 | | 191.048982 |
| 2057 | 23.801 | 1106.903 | 187048.984 | | 1384.16248 |
| 2058 | 50.557 | 1104.421 | 171816.922 | | 1271.44522 |
| 2059 | 58.615 | 1102.375 | 6383.561 | | 47.2383514 |
| 2060 | 51.593 | 1101.214 | 1985.06 | | 14.689444 |
| 2061 | 49.777 | 1100.828 | 2256.389 | | 16.6972786 |
| 2062 | 19.219 | 1100.543 | 6005.286 | | 44.4391164 |
| 2063 | 21.831 | 1100.522 | 38794.496 | | 287.07927 |
| 2064 | 25.329 | 1099.552 | 13957.11 | | 103.282614 |
| 2065 | 53.545 | 1099.417 | 30059.629 | | 222.441255 |
| 2066 | 21.401 | 1098.745 | 112194.32 | | 830.237968 |
| 2067 | 52.17 | 1098.418 | 3632.612 | | 26.8813288 |
| 2068 | 20.123 | 1097.551 | 4589.071 | | 33.9591254 |
| 2069 | 24.128 | 1096.552 | 522675.844 | | 3867.80125 |
| 2070 | 53.285 | 1096.434 | 26559.404 | | 196.53959 |
| 2071 | 51.387 | 1096.263 | 109630.906 | | 811.268704 |
| 2072 | 29.987 | 1095.392 | 58529 | | 433.1146 |
| 2073 | 19.018 | 1094.643 | 6701.293 | | 49.5895682 |
| 2074 | 20.501 | 1091.546 | 15707.116 | | 116.232658 |
| 2075 | 23.598 | 1090.488 | 10901.328 | | 80.6698272 |
| 2076 | 53.19 | 1090.433 | 338706.531 | | 2506.42833 |
| 2077 | 20.982 | 1082.62 | 65489.449 | | 484.621923 |
| 2078 | 21.397 | 1082.584 | 32035.098 | | 237.059725 |
| 2079 | 19.289 | 1080.554 | 12653.619 | | 93.6367806 |
| 2080 | 53.408 | 1076.445 | 4457.142 | | 32.9828508 |
| 2081 | 53.657 | 1076.436 | 3256.97 | | 24.101578 |
| 2082 | 52.905 | 1076.431 | 2378.771 | | 17.6029054 |
| 2083 | 20.569 | 1075.555 | 86250.758 | | 638.255609 |
| 2084 | 29.991 | 1073.413 | 40851.953 | | 302.304452 |
| 2085 | 23.494 | 1068.813 | 147886.688 | | 1094.36149 |
| 2086 | 21.443 | 1068.575 | 62297.086 | | 460.998436 |
| 2087 | 18.988 | 1068.472 | 18219.826 | | 134.826712 |
| 2088 | 23.8 | 1064.568 | 7053.475 | | 52.195715 |
| 2089 | 20.279 | 1063.503 | 21330.266 | | 157.843968 |
| 2090 | 19.116 | 1062.663 | 4397.519 | | 32.5416406 |
| 2091 | 32.024 | 1060.467 | 45340.27 | | 335.517998 |
| 2092 | 60.603 | 1060.416 | 6794.908 | | 50.2823192 |
| 2093 | 52.255 | 1058.393 | 12166.359 | | 90.0310566 |
| 2094 | 54.069 | 1058.366 | 9593.294 | | 70.9903756 |
| 2095 | 49.741 | 1057.321 | 5347.421 | | 39.5709154 |
| 2096 | 23.962 | 1054.469 | 82210.703 | | 608.359202 |
| 2097 | 23.588 | 1051.388 | 89102.742 | | 659.360291 |
| 2098 | 30.345 | 1044.353 | 4195.99 | | 31.050326 |
| 2099 | 23.401 | 1040.537 | 10620.867 | | 78.5944158 |
| 2100 | 20.577 | 1032.597 | 21339.391 | | 157.911493 |
| 2101 | 24.092 | 1025.286 | 62953.594 | | 465.856596 |
| 2102 | 19.87 | 1018.527 | 7472.115 | | 55.293651 |
| 2103 | 23.953 | 1016.515 | 121646.805 | | 900.186357 |
| 2104 | 30.418 | 1016.354 | 134689.156 | | 996.699754 |
| 2105 | 20.528 | 1015.594 | 21665.502 | | 160.324715 |
| 2106 | 23.573 | 1013.438 | 115542.805 | | 855.016757 |
| 2107 | 30.392 | 1011.276 | 33455.215 | | 247.568591 |
| 2108 | 18.922 | 1010.399 | 9148.562 | | 67.6993588 |
| 2109 | 20.423 | 1006.679 | 8503.45 | | 62.92553 |
| 2110 | 47.1 | 998.211 | 46778.898 | | 346.163845 |
| 2111 | 23.462 | 995.402 | 32905.055 | | 243.497407 |
| 2112 | 51.388 | 991.214 | 2437.004 | | 18.0338296 |
| 2113 | 21.942 | 988.585 | 32196.633 | | 238.255084 |
| 2114 | 20.878 | 988.565 | 12280.876 | | 90.8784824 |
| 2115 | 29.367 | 987.262 | 48744.594 | | 360.709996 |
| 2116 | 23.484 | 984.509 | 27134.879 | | 200.798105 |
| 2117 | 47.966 | 984.342 | 3780.828 | | 27.9781272 |
| 2118 | 23.398 | 981.665 | 20057.672 | | 148.426773 |
| 2119 | 30.895 | 981.502 | 25569.025 | | 189.210785 |
| 2120 | 19.773 | 978.532 | 4305.65 | | 31.86181 |
| 2121 | 30.386 | 973.323 | 32013.334 | | 236.898672 |
| 2122 | 29.291 | 971.253 | 5491.579 | | 40.6376846 |
| 2123 | 20.901 | 969.104 | 41305.473 | | 305.6605 |
| 2124 | 25.141 | 965.491 | 48757.68 | | 360.806832 |
| 2125 | 24.072 | 964.44 | 7556.274 | | 55.9164276 |
| 2126 | 20.829 | 963.206 | 16317.524 | | 120.749678 |
| 2127 | 28.81 | 962.49 | 19231.721 | | 142.314735 |
| 2128 | 20.219 | 960.462 | 9783.454 | | 72.3975596 |
| 2129 | 23.379 | 956.597 | 24888.623 | | 184.17581 |
| 2130 | 22.899 | 956.484 | 12570.926 | | 93.0248524 |
| 2131 | 59.245 | 955.564 | 1391.343 | | 10.2959382 |
| 2132 | 19.095 | 954.938 | 8325.797 | | 61.6108978 |
| 2133 | 20.123 | 952.494 | 5814.21 | | 43.025154 |
| 2134 | 60.591 | 950.571 | 4099.453 | | 30.3359522 |
| 2135 | 40.291 | 950.357 | 19953.055 | | 147.652607 |
| 2136 | 41.194 | 950.357 | 47130.363 | | 348.764686 |
| 2137 | 24.004 | 949.406 | 63025.402 | | 466.387975 |
| 2138 | 29.386 | 949.317 | 56691.895 | | 419.520023 |
| 2139 | 51.536 | 948.37 | 24475.225 | | 181.116665 |
| 2140 | 60.327 | 947.639 | 4107.147 | | 30.3928878 |
| 2141 | 23.951 | 945.481 | 43559.406 | | 322.339604 |
| 2142 | 20.894 | 944.588 | 148397.719 | | 1098.14312 |
| 2143 | 30.047 | 944.458 | 13261.684 | | 98.1364616 |
| 2144 | 23.976 | 933.488 | 10559.713 | | 78.1418762 |
| 2145 | 20.085 | 931.546 | 45600.914 | | 337.446764 |
| 2146 | 18.961 | 926.553 | 4596.708 | | 34.0156392 |
| 2147 | 20.898 | 925.264 | 49014.879 | | 362.710105 |
| 2148 | 20.978 | 920.557 | 5005.199 | | 37.0384726 |
| 2149 | 18.938 | 918.591 | 4865.919 | | 36.0078006 |
| 2150 | 56.883 | 918.079 | 1112.862 | | 8.2351788 |
| 2151 | 51.665 | 915.677 | 1583.223 | | 11.7158502 |
| 2152 | 52.923 | 915.395 | 2432.005 | | 17.996837 |
| 2153 | 29.904 | 914.917 | 25552.555 | | 189.088907 |
| 2154 | 40.82 | 912.413 | 454771.625 | | 3365.31003 |
| 2155 | 23.975 | 911.5 | 11979.838 | | 88.6508012 |
| 2156 | 18.986 | 911.287 | 4659.146 | | 34.4776804 |
| 2157 | 29.339 | 906.271 | 22190.25 | | 164.20785 |
| 2158 | 51.09 | 902.979 | 700.182 | | 5.1813468 |
| 2159 | 20.832 | 902.486 | 52433.75 | | 388.00975 |
| 2160 | 49.645 | 900.36 | 24044.1 | | 177.92634 |
| 2161 | 24.096 | 898.383 | 45429.156 | | 336.175754 |
| 2162 | 56.504 | 897.659 | 5498.265 | | 40.687161 |
| 2163 | 56.278 | 896.829 | 1467.63 | | 10.860462 |
| 2164 | 22.577 | 896.41 | 21323.018 | | 157.790333 |
| 2165 | 19.262 | 895.584 | 4205.946 | | 31.1240004 |
| 2166 | 23.992 | 882.405 | 17175.494 | | 127.098656 |
| 2167 | 55.786 | 880.774 | 2373.386 | | 17.5630564 |
| 2168 | 59.474 | 879.635 | 1745.675 | | 12.917995 |
| 2169 | 19.949 | 879.567 | 5377.363 | | 39.7924862 |
| 2170 | 29.754 | 876.473 | 23381.605 | | 173.023877 |
| 2171 | 42.753 | 875.379 | 114573.516 | | 847.844018 |
| 2172 | 21.028 | 872.459 | 5747.54 | | 42.531796 |
| 2173 | 19.956 | 868.588 | 5911.415 | | 43.744471 |
| 2174 | 51.593 | 867.774 | 1445.617 | | 10.6975658 |
| 2175 | 55.321 | 867.675 | 724.974 | | 5.3648076 |
| 2176 | 59.198 | 867.635 | 1190.664 | | 8.8109136 |
| 2177 | 58.799 | 867.622 | 2502.561 | | 18.5189514 |
| 2178 | 20.829 | 852.468 | 7367.667 | | 54.5207358 |
| 2179 | 49.047 | 846.946 | 1713.276 | | 12.6782424 |
| 2180 | 57.438 | 846.946 | 2112.215 | | 15.630391 |
| 2181 | 60.566 | 846.924 | 2585.42 | | 19.132108 |
| 2182 | 22.644 | 846.426 | 1215.356 | | 8.9936344 |
| 2183 | 29.086 | 841.303 | 8721.339 | | 64.5379086 |
| 2184 | 53.187 | 837.301 | 11143.638 | | 82.4629212 |
| 2185 | 55.22 | 835.737 | 611.264 | | 4.5233536 |
| 2186 | 54.381 | 832.253 | 1154.893 | | 8.5462082 |
| 2187 | 59.347 | 824.672 | 836.045 | | 6.186733 |
| 2188 | 20.776 | 824.421 | 2193.866 | | 16.2346084 |
| 2189 | 59.758 | 824.025 | 5333.992 | | 39.4715408 |
| 2190 | 58.792 | 823.646 | 7006.314 | | 51.8467236 |
| 2191 | 53.249 | 821.319 | 3106.751 | | 22.9899574 |
| 2192 | 60.055 | 815.685 | 1288.741 | | 9.5366834 |
| 2193 | 29.084 | 815.416 | 2310.018 | | 17.0941332 |
| 2194 | 50.654 | 813.317 | 13369.253 | | 98.9324722 |
| 2195 | 53.192 | 799.334 | 37386.438 | | 276.659641 |

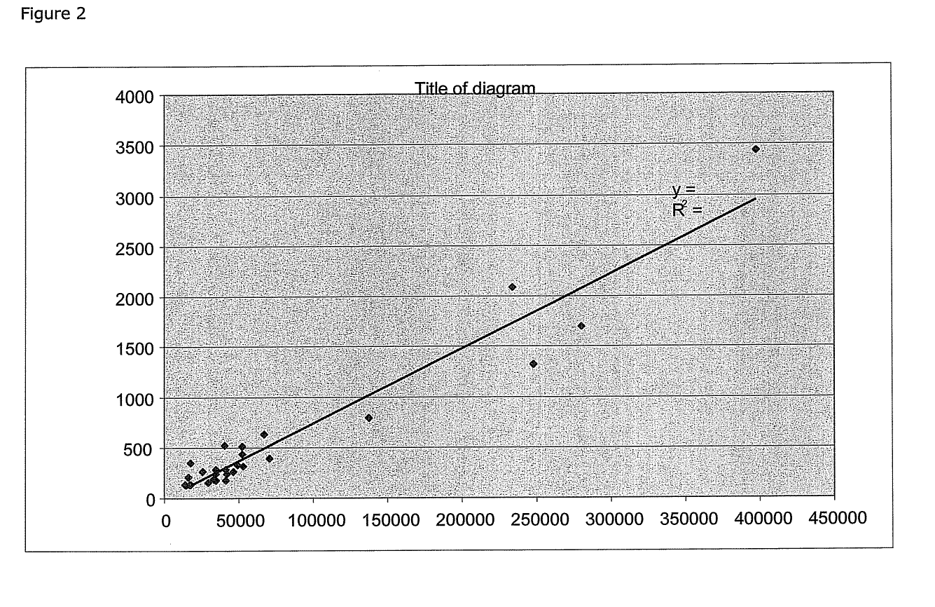

The quality of the regression is shown in FIG. 2.

1. A process for normalizing the concentration of at least one analyte in a urine sample, comprising the following steps:
a) determining the concentration of at least one analyte;
b) determining the concentration of at least one collagen or collagen fragment;
c) normalizing the concentration of said at least one analyte relative to the concentration of said at least one collagen or collagen fragment.

2. The process according to claim 1, wherein the urine sample is subdivided into several subsamples before the concentration of the analyte is determined.

3. The process according to claim 2, wherein said separation is effected by:
chromatographic methods, such as affinity chromatography, ion-exchange chromatography, reversed-phase chromatography, hydrophobic interaction chromatography, gel filtration, optionally using FPLC or HPLC;
adsorption to specific materials, such as SELDI process, ClinProt process;
electrophoretic methods, such as gel electrophoresis or capillary electrophoresis.

4. The process according to claim 1, wherein said determination of the contents of analytes and/or collagen or collagen fragments in said subsamples is effected by a UV/Vis detector, fluorescence detector, refractive index detector, diode array detector, mass spectrometer, gel scanner, staining or derivatization, followed by detection with suitable means.

5. The process according to claim 1, wherein said at least one collagen or collagen fragment is identified by its migration or retention time and optionally further suitable parameters, such as mass spectra, UV/Vis spectra, fluorescence spectra, affinity for particular ligands, for example, antibodies.

6. The process according to claim 1, wherein said at least one collagen fragment is selected from the peptides of SEQ ID Nos. 1 to 42.

7. The process according to claim 1, wherein the concentrations of at least two or at least three, preferably at least 5, 10, 20, 30, 40 or 50 collagens or collagen fragments are determined and employed for normalization.

8. The process according to claim 1, wherein said normalization is effected by linear regression.

9. Use of collagens or collagen fragments for the normalization of analyte concentrations in urine samples.

10. The use according to claim 9, wherein said collagen fragments are selected from the peptides of SEQ ID Nos. 1 to 42.

11. The use according to claim 9, wherein at least two or at least three, preferably at least 5, 10, 20, 30, 40 or 50 collagens or collagen fragments are employed for normalization.
